(19) United States
(12) Patent Application Publication
(10) Pub. No.: US 2017/0014495 A1
(43) Pub. Date: Jan. 19, 2017

(54) ENOLASE 1 (ENO1) COMPOSITIONS AND USES THEREOF

(71) Applicant: Berg LLC, Nashville, TN (US)

(72) Inventors: Rangaprasad Sarangarajan, Boylston, MA (US); Vivek K. Vishnudas, Bedford, MA (US); Alexander Harrison Taylor, Mount Juliet, TN (US)

(21) Appl. No.: 15/213,300

(22) Filed: Jul. 18, 2016

Related U.S. Application Data
(60) Provisional application No. 62/193,582, filed on Jul. 16, 2015.
(60) Provisional application No. 62/207,152, filed on Aug. 19, 2015.
(60) Provisional application No. 62/235,854, filed on Oct. 1, 2015.

(51) Int. Cl. A61K 38/51; A61K 38/08; A61K 47/48; C12N 9/88; C07K 7/06

(52) U.S. Cl. CPC A61K 38/51; C12N 9/88; C12Y 402/01011; C07K 7/06; A61K 47/48215; A61K 38/08; C07K 2319/50; C07K 2319/33

(57) ABSTRACT

The invention provides compositions comprising Eno1 and a muscle targeting peptide, e.g, as a fusion protein, for delivery of Eno1 to a muscle. The Eno1 may contain one or more added cysteine residues which are covalently attached to a biocompatible polymer (e.g. polyethylene glycol). Further, the invention provides a method for normalizing blood glucose in a subject with elevated blood glucose, comprising administering to the subject enolase 1 (Eno1), thereby normalizing blood glucose in the subject. The invention also provides methods of treating one or more conditions including impaired glucose tolerance, insulin resistance, pre-diabetes, and diabetes, especially type 2 diabetes in a subject, comprising administering to the subject enolase 1 (Eno1), thereby treating the condition in the subject.

### RELATED APPLICATIONS

This application claims priority to U.S. Provisional Patent Application No. 62/193,582 filed on Jul. 16, 2015; U.S. Provisional Patent Application No. 62/207,152 filed on Aug. 19, 2015, and U.S. Provisional Patent Application No. 62/235,854 filed on Oct. 1, 2015, the contents of each of which are incorporated herein in their entirety.

### SUBMISSION OF SEQUENCE LISTING

The Sequence Listing associated with this application is filed in electronic format via EFS-Web and hereby incorporated by reference into the specification in its entirety. The name of the text file containing the Sequence Listing is 119992_14904_Sequence_Listing. The size of the text file is 40 KB, and the text file was created on Jul. 16, 2016.

### BACKGROUND

As the levels of blood glucose rise postprandially, insulin is secreted and stimulates cells of the peripheral tissues (skeletal muscles and fat) to actively take up glucose from the blood as a source of energy. Loss of glucose homeostasis as a result of dysregulated insulin secretion or action typically results in metabolic disorders such as diabetes, which may be co-triggered or further exacerbated by obesity. Because these conditions can reduce the quality of life or even be fatal, strategies to restore adequate glucose clearance from the bloodstream are required.

Although diabetes may arise secondary to any condition that causes extensive damage to the pancreas (e.g., pancreatitis, tumors, administration of certain drugs such as corticosteroids or pentamidine, iron overload (i.e., hemochromatosis), acquired or genetic endocrinopathies, and surgical excision), the most common forms of diabetes typically arise from primary disorders of the insulin signaling system. There are two major types of diabetes, namely type 1 diabetes (also known as insulin dependent diabetes (IDDM)) and type 2 diabetes (also known as insulin independent or non-insulin dependent diabetes (NIDDM)), which share common long-term complications in spite of their different pathogenic mechanisms.

Type 1 diabetes, which accounts for approximately 10% of all cases of primary diabetes, is an organ-specific autoimmune disease characterized by the extensive destruction of the insulin-producing beta cells of the pancreas. The consequent reduction in insulin production inevitably leads to the deregulation of glucose metabolism. While the administration of insulin provides significant benefits to patients suffering from this condition, the short serum half-life of insulin is a major impediment to the maintenance of normoglycemia. An alternative treatment is islet transplantation, but this strategy has been associated with limited success.

Type 2 diabetes, which affects a larger proportion of the population, is characterized by a deregulation in the secretion of insulin and/or a decreased response of peripheral tissues to insulin, i.e., insulin resistance. While the pathogenesis of type 2 diabetes remains unclear, epidemiologic studies suggest that this form of diabetes results from a collection of multiple genetic defects or polymorphisms, each contributing its own predisposing risks and modified by environmental factors, including excess weight, diet, inactivity, drugs, and excess alcohol consumption. Although various therapeutic treatments are available for the management of type 2 diabetes, they are associated with various debilitating side effects. Accordingly, patients diagnosed with or at risk of having type 2 diabetes are often advised to adopt a healthier lifestyle, including loss of weight, change in diet, exercise, and moderate alcohol intake. Such lifestyle changes, however, are not sufficient to reverse the vascular and organ damages caused by diabetes.

### SUMMARY OF THE INVENTION

In one aspect, the invention relates to an Eno1 molecule comprising an Eno1 polypeptide or a fragment thereof and a muscle targeting peptide, wherein the Eno1 polypeptide or fragment thereof is covalently attached to the muscle targeting peptide. In certain embodiments, the molecule is for delivery to a muscle cell. In certain embodiments, the Eno1 polypeptide or fragment thereof is biologically active. In certain embodiments, the Eno1 polypeptide or fragment thereof has at least 90% of the activity of a purified endogenous human Eno1 polypeptide. In certain embodiments, the Eno1 polypeptide or fragment thereof is human Eno1 or a fragment thereof. In certain embodiments, the muscle targeting peptide comprises an amino acid sequence selected from the group consisting of: ASSLNIA (SEQ ID NO: 7); WDANGKT (SEQ ID NO: 8); GETRAPL (SEQ ID NO: 9); CGHHPVYAC (SEQ ID NO: 5); and HAIYPRH (SEQ ID NO: 6).

In certain embodiments, the Eno1 molecule further comprises a linker. In certain embodiments, the linker is selected from the group consisting of a covalent linker, a non-covalent linkage, and a reversible linker. In certain embodiments, the linker is attached to the C-terminus of the Eno1 polypeptide or fragment thereof. In a preferred embodiment, the linker is attached to the N-terminus of the Eno1 polypeptide or fragment thereof. In certain embodiments, the muscle targeting peptide is attached to the N-terminus of the linker. In certain embodiments, the linker is a peptide comprising a protease cleavage site. In certain embodiments, the linker comprises the amino acid sequence of SEQ ID NO: 6.

In certain embodiments, the Eno1 polypeptide or fragment thereof and the muscle targeting peptide are comprised in a single polypeptide.

In certain embodiments of the aforementioned Eno1 molecules, the Eno1 molecule further comprises one or more functional moiety. In certain embodiments, the Eno1 polypeptide or fragment thereof is covalently attached to the one or more functional moiety. In certain embodiments, the Eno1 polypeptide, or fragment thereof, comprises one or more cysteine residues covalently attached to the one or more functional moiety. In certain embodiments, the Eno1 polypeptide, or fragment thereof, comprises two cysteine residues covalently attached to the one or more functional moiety. In certain embodiments, the Eno1 polypeptide, or fragment thereof comprises three cysteine residues covalently attached to the one or more functional moiety. In certain embodiments, the cysteine residues are added cysteine residues. In certain embodiments, the cysteine residues are at a position selected from the group consisting of position 26, 78, 140, 236, 253, 267 and 418 of the amino acid sequence of SEQ ID NO: 13. In certain embodiments, the Eno1 polypeptide or fragment thereof is released from the muscle targeting peptide or the one or more functional moiety upon delivery to a muscle cell.

In certain embodiments, the one or more functional moiety is a moiety selected from the group consisting of a biocompatible polymer, a cell penetrating peptide, and a muscle targeting peptide. In certain embodiments, the functional moiety is a biocompatible polymer. In certain embodiments, the biocompatible polymer comprises polyethylene glycol (PEG). In certain embodiments, the PEG is a linear PEG or a branched PEG. In certain embodiments, the PEG is a 5 kDa PEG, 10 kDa PEG, or 20 kDa PEG. In certain embodiments, the single polypeptide comprises the amino acid sequence of SEQ ID NO: 16 comprising an added cysteine residue at position 289, wherein the added cysteine residue at position 289 is covalently linked to at least one PEG molecule. In certain embodiments, the added cysteine residue is covalently linked to the PEG molecule through a maleimide linkage.

In certain aspects, the present invention also relates to a pharmaceutical composition comprising any of the Eno1 molecules described above.

In certain aspects, the present invention also relates to a nucleic acid encoding any one of the Eno1 molecules described above. In certain aspects, the present invention also relates to an expression vector comprising the nucleic acid.

In certain aspects, the present invention also relates to an Eno1 molecule comprising an Eno1 polypeptide or a fragment thereof, wherein the Eno1 polypeptide or fragment thereof comprises at least one added cysteine residue. In certain embodiments, the Eno1 polypeptide or fragment thereof comprises at least 2 added cysteine residues. In certain embodiments, the Eno1 polypeptide or fragment thereof comprises at least 3 added cysteine residues. In certain embodiments, the added cysteine residue is added to the N-terminus of the Eno1 polypeptide or fragment thereof. In certain embodiments, the added cysteine residue is added to the C-terminus of the Eno1 polypeptide or fragment thereof. In certain embodiments, the added cysteine residue replaces an internal serine or threonine of the Eno1 polypeptide or fragment thereof. In certain embodiments, the added cysteine residue is at one or more positions selected from the group consisting of position 26, 78, 140, 236, 253, 267 and 418 of the amino acid sequence of SEQ ID NO: 13.

In certain embodiments, the Eno1 molecule further comprises a functional moiety. In certain embodiments, the functional moiety is a cell penetrating peptide. In certain embodiments, the functional moiety is a muscle targeting peptide. In certain embodiments, the muscle targeting peptide comprises an amino acid sequence selected from the group consisting of: ASSLNIA (SEQ ID NO: 7); WDANGKT (SEQ ID NO: 8); GETRAPL (SEQ ID NO: 9); CGHHPVYAC (SEQ ID NO: 5); and HAIYPRH (SEQ ID NO: 6). In certain embodiments, the Eno1 polypeptide or fragment thereof and the muscle targeting peptide are comprised in a single polypeptide. In certain embodiments, the Eno1 molecule comprises a polypeptide linker between the Eno1 polypeptide or fragment thereof and the muscle targeting peptide. In certain embodiments, the polypeptide linker comprises the amino acid sequence of SEQ ID NO. 6.

In certain embodiments of the aforementioned Eno1 molecules, the functional moiety is a biocompatible polymer. In certain embodiments, the biocompatible polymer comprises polyethylene glycol (PEG). In certain embodiments, the PEG is a linear PEG or a branched PEG. In certain embodiments, the PEG is a 5 kDa PEG, 10 kDa PEG, or 20 kDa PEG. In certain embodiments, the Eno1 molecule comprises a linker between the functional moiety and the Eno1 polypeptide or fragment thereof. In certain embodiments, the linker is attached to the Eno1 polypeptide or fragment thereof at the added cysteine residue. In certain embodiments, the linker comprises the amino acid sequence of SEQ ID NO: 14. In certain embodiments, the N-terminus of the linker is attached to the Eno1 polypeptide or fragment thereof at the added cysteine residue. In certain embodiments, the single polypeptide comprises the amino acid sequence of SEQ ID NO: 16 comprising an added cysteine residue at position 289, wherein the added cysteine residue at position 289 is covalently linked to at least one PEG molecule through a maleimide linkage. In certain embodiments, the at least one PEG molecule is a linear 20 kDa PEG.

In certain aspects, the invention also relates to a pharmaceutical composition comprising any of the Eno1 molecules described above.

In certain aspects, the invention also relates to a nucleic acid encoding any of the aforementioned Eno1 molecules. In certain aspects, the invention also relates to an expression vector comprising the nucleic acid.

In certain embodiments of the aforementioned pharmaceutical compositons, the composition is formulated for parenteral administration. In certain embodiments, the composition is formulated for oral administration. In certain embodiments, the composition is formulated for intramuscular administration, intravenous administration, or subcutaneous administration.

In certain aspects, the invention also relates to a method of decreasing blood glucose in a subject with elevated blood glucose, the method comprising administering to the subject any of the aforementioned pharmaceutical compositions, thereby decreasing blood glucose in the subject.

In certain aspects, the invention also relates to a method of increasing glucose tolerance in a subject with decreased glucose tolerance, the method comprising administering to the subject any of the aforementioned pharmaceutical compositions, thereby increasing glucose tolerance in the subject.

In certain aspects, the invention also relates to a method of improving insulin response in a subject with decreased insulin sensitivity and/or insulin resistance, the method comprising administering to the subject any of the aforementioned pharmaceutical compositions, thereby improving insulin response in the subject.

In certain aspects, the invention also relates to a method of treating diabetes in a subject, the method comprising administering to the subject any of the aforementioned pharmaceutical compositions, thereby treating diabetes in the subject. In certain embodiments, the diabetes is type 2 diabetes or type 1 diabetes. In certain embodiments, the diabetes is pre-diabetes.

In certain aspects, the invention also relates to a method of decreasing an HbA1c level in a subject with an elevated Hb1Ac level, the method comprising administering to the subject any of the aforementioned pharmaceutical compositions, thereby decreasing the HbA1c level in the subject.

In certain aspects, the invention also relates to a method of improving blood glucose level control in a subject with abnormal blood glucose level control, the method comprising administering to the subject any of the aforementioned pharmaceutical compositions, thereby improving blood glucose level control in the subject.

In certain embodiments of the aforementioned methods, glucose flux in a skeletal muscle cell of the subject is increased.

In certain aspects, the invention also relates to a method of increasing glucose flux in a subject, the method comprising administering to the subject any of the aforementioned pharmaceutical compositions thereby increasing glucose flux in the subject.

In certain aspects, the invention also relates to a method of increasing glycolytic activity or capacity in a skeletal muscle cell of a subject, the method comprising administering to the subject any of the aforementioned pharmaceutical compositions, thereby increasing glycolytic activity or capacity in a skeletal muscle cell of the subject.

In certain aspects, the invention also relates to a method of increasing mitochondrial free fatty acid oxidation in a skeletal muscle cell of a subject, the method comprising administering to the subject any of the aforementioned pharmaceutical compositions, thereby increasing mitochondrial free fatty acid oxidation in a skeletal muscle cell of the subject.

In certain embodiments of the aforementioned methods, the Eno1 is administered parenterally. In certain embodiments of the aforementioned methods, the Eno1 is administered orally. In certain embodiments of the aforementioned methods, the Eno1 is administered by a route selected from the group consisting of intramuscular, intravenous, and subcutaneous. In certain embodiments of the aforementioned methods, the subject has any one or more of elevated blood glucose, decreased glucose tolerance, decreased insulin sensitivity and/or insulin resistance, diabetes, elevated Hb1Ac level, and abnormal blood glucose level control. In certain embodiments of the aforementioned methods, the method further comprises selecting a subject having any one or more of elevated blood glucose, decreased glucose tolerance, decreased insulin sensitivity and/or insulin resistance, diabetes, elevated Hb1Ac level, and abnormal blood glucose level control. In certain embodiments of the aforementioned methods, the subject is human.

In one aspect, the invention relates to a pharmaceutical composition comprising Eno1 or a fragment thereof and a muscle targeting peptide. In another aspect, the invention relates to a pharmaceutical composition comprising Eno1, or a fragment thereof, a muscle targeting peptide, and one or more PEG groups. In certain embodiments, the composition is for delivery to a muscle cell. In certain embodiments, the Eno1 comprises an Eno1 polypeptide or a fragment thereof. In certain embodiments, the Eno1 comprises an Eno1 nucleic acid or a fragment thereof. In certain embodiments, the composition further comprises an expression vector encoding the Eno1 or fragment thereof. In certain embodiments, the Eno1 or fragment thereof is biologically active. In certain embodiments, the Eno1 or fragment thereof has at least 90% of the activity of a purified endogenous human Eno1 polypeptide. In certain embodiments, the Eno1 is human Eno1. In certain embodiments, the composition further comprises a liposome. In certain embodiments, the composition comprises a complex comprising the Eno1 polypeptide or fragment thereof and a muscle targeting peptide. In certain embodiments, the Eno1 polypeptide is human Eno1 polypeptide. In certain embodiments, the muscle targeting peptide comprises an amino acid sequence selected from the group consisting of: ASSLNIA (SEQ ID NO: 7); WDANGKT (SEQ ID NO: 8); GETRAPL (SEQ ID NO: 9); CGHHPVYAC (SEQ ID NO: 5); and HAIYPRH (SEQ ID NO: 6). In certain embodiments, the complex further comprises a linker. In certain embodiments, the linker is selected from the group consisting of a covalent linker, a non-covalent linkage, and a reversible linker. In certain embodiments, the linker is attached to the N-terminus of the Eno1 polypeptide or fragment thereof. In certain embodiments, the muscle targeting peptide is attached to the N-terminus of the linker. In certain embodiments, the linker comprises the amino acid sequence of SEQ ID NO: 6. In certain embodiments, the Eno1 is released from the complex upon delivery to a muscle cell.

In certain aspects, the invention relates to a pharmaceutical composition comprising a complex comprising an Eno1 protein, wherein the Eno1 protein comprises at least one added cysteine residue. In certain embodiments, the Eno1 protein or fragment thereof comprises at least 2 added cysteine residues. In certain embodiments, the Eno1 protein or fragment thereof comprises at least 3 added cysteine residues. In certain embodiments, the added cysteine residue is added to the N-terminus of the Eno1 protein. In certain embodiments, the added cysteine residue replaces an internal serine or threonine of the Eno1 protein. In certain embodiments, the complex comprising an Eno1 protein with at least one added cysteine residue has at least one cysteine linked to a PEG group. In certain embodiments, the complex comprising an Eno1 protein with at least two cysteine residues has at least two cysteines linked to a PEG group. In certain embodiments, the complex comprising an Eno1 protein with at least three cysteine residues has at least three cysteines linked to a PEG group. In certain embodiments, the complex further comprises a functional moiety. In certain embodiments, the functional moiety is a cell penetrating peptide. In certain embodiments, the functional moiety is a muscle targeting peptide. In certain embodiments, the muscle targeting peptide comprises an amino acid sequence selected from the group consisting of: ASSLNIA (SEQ ID NO: 7); WDANGKT (SEQ ID NO: 8); GETRAPL (SEQ ID NO: 9); CGHHPVYAC (SEQ ID NO: 5); and HAIYPRH (SEQ ID NO: 6). In certain embodiments, the complex further comprises a linker between the functional moiety and the Eno1 protein. In certain embodiments, the linker is attached to the Eno1 protein at the added cysteine residue. In certain embodiments, the linker comprises the amino acid sequence of SEQ ID NO: 14. In certain embodiments, the N-terminus of the linker is attached to the Eno1 protein at the added cysteine residue.

In certain embodiments of the aforementioned pharmaceutical compositions, the composition is formulated for parenteral administration. In certain embodiments, the composition is formulated for oral administration. In certain embodiments, the composition is formulated for intramuscular administration, intravenous administration, or subcutaneous administration.

In certain aspects, the invention relates to a method of decreasing blood glucose in a subject with elevated blood glucose, the method comprising administering to the subject any of the aforementioned pharmaceutical compositions, thereby decreasing blood glucose in the subject.

In certain aspects, the invention relates to a method of increasing glucose tolerance in a subject with decreased glucose tolerance, the method comprising administering to the subject any of the aforementioned pharmaceutical compositions, thereby increasing glucose tolerance in the subject.

In certain aspects, the invention relates to a method of improving insulin response in a subject with decreased insulin sensitivity and/or insulin resistance, the method comprising administering to the subject any of the aforementioned pharmaceutical compositions, thereby improving insulin response in the subject.

In certain aspects, the invention relates to a method of treating diabetes in a subject, the method comprising administering to the subject any of the aforementioned pharmaceutical compositions, thereby treating diabetes in the subject. In certain embodiments, the diabetes is type 2 diabetes or type 1 diabetes. In certain embodiments, the diabetes is pre-diabetes.

In certain aspects, the invention relates to a method of decreasing an HbA1c level in a subject with an elevated Hb1Ac level, the method comprising administering to the subject any of the aforementioned pharmaceutical compositions, thereby decreasing the HbA1c level in the subject.

In certain aspects, the invention relates to a method of improving blood glucose level control in a subject with abnormal blood glucose level control, the method comprising administering to the subject any of the aforementioned pharmaceutical compositions, thereby improving blood glucose level control in the subject. In certain embodiments of the aforementioned methods, glucose flux in a skeletal muscle cell of the subject is increased.

In certain aspects, the invention relates to a method of increasing glucose flux in a subject, the method comprising administering to the subject any of the aforementioned pharmaceutical compositions, thereby increasing glucose flux in the subject.

In certain aspects, the invention relates to a method of increasing glycolytic activity or capacity in a skeletal muscle cell of a subject, the method comprising administering to the subject any of the aforementioned pharmaceutical compositions, thereby increasing glycolytic activity or capacity in a skeletal muscle cell of the subject.

In certain aspects, the invention relates to a method of increasing mitochondrial free fatty acid oxidation in a skeletal muscle cell of a subject, the method comprising administering to the subject any of the aforementioned pharmaceutical compositions, thereby increasing mitochondrial free fatty acid oxidation in a skeletal muscle cell of the subject. In certain embodiments, the Eno1 is administered parenterally. In certain embodiments, the Eno1 is administered orally. In certain embodiments, the Eno1 is administered by a route selected from the group consisting of intramuscular, intravenous, and subcutaneous. In certain embodiments, the subject has any one or more of elevated blood glucose, decreased glucose tolerance, decreased insulin sensitivity and/or insulin resistance, diabetes, elevated Hb1Ac level, and abnormal blood glucose level control. In certain embodiments, the method further comprises selecting a subject having any one or more of elevated blood glucose, decreased glucose tolerance, decreased insulin sensitivity and/or insulin resistance, diabetes, elevated Hb1Ac level, and abnormal blood glucose level control. In certain embodiments, the subject is human.

In one aspect, the invention provides a method of treating obesity in a subject in need thereof, comprising administering to the subject a therapeutically effective amount of a composition comprising Eno1 or a fragment thereof, thereby treating obesity in the subject. In certain embodiments, the subject is suffering from obesity, and the obesity is type 2 diabetes, type 1 diabetes, or pre-diabetes. In certain embodiments, the obesity is caused by a therapeutic treatment. In certain embodiments, the therapeutic treatment is a diabetic drug.

In one aspect, the invention provides a method of reducing body weight in a subject afflicted with an overweight condition, comprising administering to the subject a therapeutically effective amount of a composition comprising Eno1 or a fragment thereof, thereby reducing body weight in the subject. In certain embodiments, the subject has a body mass index of between 25 kg/m² and 30 kg/m². In certain embodiments, the overweight condition is caused by a therapeutic treatment. In certain embodiments, the therapeutic treatment is a diabetic drug.

In one aspect, the invention provides a method of reducing or preventing body weight gain in a subject, comprising administering to the subject a therapeutically effective amount of a composition comprising Eno1 or a fragment thereof, thereby reducing or preventing body weight gain in the subject. In certain embodiments, the subject is in need of a therapeutic treatment that induces weight gain. In certain embodiments, the subject is undergoing a therapeutic treatment that induces weight gain. In certain embodiments, the therapeutic treatment is a diabetic drug. In certain embodiments, the diabetic drug is selected from the group consisting of sulfonylureas, insulin, GLP-1 receptor agonists, DPP-4 inhibitors, metformin, and rosiglitazone. In certain embodiments, the diabetic drug is rosiglitazone. In certain embodiments, the subject is afflicted with diabetes. In certain embodiments, the diabetes is type 2 diabetes, type 1 diabetes, or pre-diabetes.

In certain embodiments of the aforementioned methods, administering Eno1 to the subject reduces body weight by at least 5% relative to a control. In certain embodiments, administering Eno1 to the subject reduces body mass index (BMI) by at least 5% relative to a control. In certain embodiments, the subject has any one or more of elevated blood glucose, decreased glucose tolerance, decreased insulin sensitivity and/or insulin resistance, diabetes, elevated Hb1Ac level, and abnormal blood glucose level control. In certain embodiments, the method further comprises selecting a subject having any one or more of obesity, elevated blood glucose, decreased glucose tolerance, decreased insulin sensitivity and/or insulin resistance, diabetes, elevated Hb1Ac level, and abnormal blood glucose level control. In certain embodiments, the subject is human. In certain embodiments, the Eno1 or fragment thereof comprises an Eno1 polypeptide or a fragment thereof. In certain embodiments, the Eno1 or fragment thereof comprises an Eno1 nucleic acid or a fragment thereof. In certain embodiments, the Eno1 nucleic acid or fragment thereof is present in an expression vector. In certain embodiments, the Eno1 or fragment thereof is biologically active. In certain embodiments, the Eno1 or fragment thereof has at least 90% activity of a purified endogenous human Eno1 polypeptide. In certain embodiments, the Eno1 is human Eno1.

In certain embodiments of the aforementioned methods, the composition comprising Eno1 or a fragment thereof is for delivery to a muscle cell. In certain embodiments, the composition further comprises a muscle targeting moiety. In certain embodiments, the muscle targeting moiety is a muscle targeting peptide. In certain embodiments, the Eno1 polypeptide or fragment thereof and the muscle targeting peptide are present in a complex. In certain embodiments, the muscle targeting peptide comprises an amino acid sequence selected from the group consisting of: ASSLNIA (SEQ ID NO: 7); WDANGKT (SEQ ID NO: 8); GETRAPL (SEQ ID NO: 9); CGHHPVYAC (SEQ ID NO: 5); and HAIYPRH (SEQ ID NO: 6). In certain embodiments, the complex further comprises a linker. In certain embodiments, the linker is selected from the group consisting of a covalent linker, a non-covalent linkage, and a reversible linker. In certain embodiments, the linker comprises a protease cleavage site. In certain embodiments, the Eno1 is released from the complex upon delivery to a muscle cell. In certain embodiments, the Eno1 and the muscle targeting peptide are present in the complex at a ratio of about 1:1 to about 1:30. In certain embodiments, the composition further comprises a liposome. In certain embodiments, the Eno1 is administered orally. In certain embodiments, the Eno1 is administered parenterally. In certain embodiments, the Eno1 is administered by a route selected from the group consisting of intramuscular, intravenous, and subcutaneous.

Other embodiments are provided infra.

### BRIEF DESCRIPTION OF THE DRAWINGS

FIG. 1 shows SDS-PAGE and densitometric analysis of the purified native human Eno1 protein (Enolase A, EnoA).

FIG. 2 shows size exclusion analysis of the pooled purified native human Eno1 protein. The single uniform peak indicates the purity of the protein.

FIG. 3 shows a dynamic light scattering (DLS) histogram of an Eno1 fusion protein containing an N-terminal muscle targeting peptide (ASSLNIA, SEQ ID NO: 7) in PBS buffer, pH 7.4. Dynamic light scattering provides an estimate of the globular size of the protein.

FIG. 4 shows the results of MALDI-TOF analysis of purified native human Eno1 protein. A primary peak (MH+) was observed at 47,009 Da, an MH2+ peak at 23,517.4 Da, and an MH3+ peak at 15,681.4 Da. This molecular weight matches that of untagged human Eno1 in which the N-terminal methionine residue has been removed.

FIG. 5 shows fed blood glucose levels in a leptin receptor mutation (db/db) mouse model of diabetes. Mice were dosed twice daily at 12 hour intervals by intravenous injection with saline (control), 400 μg/kg/day Eno1-SMTP fusion protein, or 800 μg/kg/day Eno1-SMTP fusion protein. Fed blood glucose was measured immediately before the morning injection, i.e. approximately 12 hours after the previous evening injection.

FIGS. 6A-6D show the levels of human Eno1 in serum (6A), liver (6B), muscle (6C) and kidney (6D) in db/db mice after 22 days of treatment with the Eno1-SMTP fusion protein or saline (control). Eno1 levels were detected by ELISA using a polyclonal anti-Eno1 antibody. The amount of Eno1 detected in the saline-treated mice was subtracted.

FIG. 7 shows the three dimensional structure of the human Eno1 dimer orienting the dimer along the axis of symmetry with the N-terminus at the top. This structure indicates that serine residues S26 and S78 are at the top of the dimer and point in the same direction; serine residues S140 and S418 are in the middle of the dimer (near the C-terminus) and point in opposite directions; and residues S236, S253 and S267 are at the bottom of the dimer and point in the same direction. Position numbering is based on the human Eno1 sequence with the N-terminal methionine removed (SEQ ID NO: 13).

FIGS. 8A and 8B show the (A) amino acid (SEQ ID NO: 2) and (B) nucleic acid coding sequence (SEQ ID NO: 1) of human Eno1, variant 1 (NCBI Accession No. NM_001428.3).

FIGS. 9A and 9B show the (A) amino acid (SEQ ID NO: 4) and (B) nucleic acid coding sequence (SEQ ID NO: 3) of human Eno1, variant 2 (NCBI Accession No. NM_001201483.1). The human Eno1, variant 2 protein is also referred to as MBP-1.

FIG. 10 shows the amino acid sequence of human Eno1, variant 1 from FIG. 8A with the N-terminal methionine removed (SEQ ID NO: 13). The serines at positions 140, 267 and 418 are shown in bold and underlined.

FIGS. 11A, 11B and 11C show fed blood glucose levels in a leptin receptor mutation (db/db) mouse model of diabetes. Mice were dosed once daily for 3 days by intravenous (IV) injection with saline (control), 0.4 mg/kg/day Eno1-SMTP fusion protein (Eno1), or 1.6 mg/kg/day Eno1-SMTP fusion protein (Eno1). Fed blood glucose was measured immediately before the injection on the third day and 1, 2, 4, 6, 10 and 24 hours after the injection on the third day. FIG. 11A shows the average glucose level of three mice. FIG. 11B shows glucose levels as a percentage of the initial value before Eno1 injection on the third day (% of Baseline). FIG. 11C shows glucose levels as a percentage of the saline control (% of Saline).

FIGS. 12A and 12B show fed blood glucose levels in a leptin receptor mutation (db/db) mouse model of diabetes. Mice were dosed once daily for 3 days by intraperitoneal (IP) injection with saline (control) or 1.6 mg/kg/day Eno1-SMTP fusion protein (Eno1). Fed blood glucose was measured immediately before the injection on the third day and 1, 2, 4, 6, 10 and 24 hours after the injection (Time After TX) on the third day. FIG. 12A shows the average glucose level of three mice. FIG. 12B shows glucose levels as a percentage of the initial value before Eno1 injection on the third day (% of baseline).

FIG. 13A shows fed blood glucose levels in db/db mice. Mice were dosed twice daily by intraperitoneal injection with saline (control) or escalating doses of Enolase-1+SMTP fusion protein (100, 200, 400, 600, 800, 1200 or 1600 μg/kg/day). Fed blood glucose was measured daily before injection. FIG. 13B shows fasted blood glucose levels in the db/db mice.

FIGS. 14A and 14B show the levels of human Eno1 in serum (14A) and skeletal muscle, liver, kidney, subcutaneous fat and visceral fat (14B) in db/db mice after 22 days of treatment with the Eno1-SMTP fusion protein or saline (control). Eno1 levels were detected by ELISA using a polyclonal anti-Eno1 antibody. The amount of Eno1 detected in the saline-treated mice was subtracted from the amount in the Eno1 treated mice.

FIG. 15 shows the three dimensional structure of monomeric human Eno1. Exemplary positions of serine residues that may be substituted with cysteines (e.g. S26C, S140C, S267C and S418C) are shown.

FIGS. 16A, 16B and 16C show fed blood glucose levels in db/db mice intravenously administered a saline control or 1.6 mg/kg/day of a cysteine modified Eno1-MTP fusion protein conjugated to PEG. Three different cysteine modified Eno1-MTP fusion proteins were evaluated in which a serine residue at position 140 (Enolase-1+SMTP-140-PEG20K), position 267 (Enolase-1+SMTP-257-PEG20K) or position 418 (Enolase-1+SMTP-418-PEG20K) of SEQ ID NO: 13 was replaced with a cysteine residue. The added cysteine residue was conjugated to linear 20 kD PEG with a maleimide linkage. Fed blood glucose was measured before injection (Pre) and 2 hours and 6 hours after injection.

FIG. 17 shows the amino acid sequence of a cysteine modified Eno1-MTP fusion protein (S267C) (SEQ ID NO: 16). The fusion protein comprises human Eno1, transcript variant 1, with the N-terminal methionine removed. A serine residue at position 267 of the Eno1 protein (SEQ ID NO: 13) was replaced with a cysteine residue. The MTP peptide (ASSLNIA, SEQ ID NO: 7) is shown in bold and underlined, and the protease tag (SSGVDLGTENLYFQ, SEQ ID NO: 6) is shown in bold. The peptide GIEGR (SEQ ID NO: 15) was added to the C-terminus of the Eno1 protein.

FIG. 18 shows the effect of rosiglitazone and Eno1 on body weight in a diabetic mouse model (db/db mice). Treatment groups shown are Saline_Lean (saline treatment of lean mice); Saline-db (saline treatment of db/db mice); Rosi (rosiglitazone treatment of db/db mice, 20 mg/kg/day); and Rosi+Eno1 (combination of 20 mg/kg/day rosiglitazone and 400 μg/kg/day Eno1 treatment of db/db mice). Rosiglitazone alone and rosiglitazone+Eno1 showed increased body weight compared to control (saline treated) db/db mice. However, body weight was lower in the rosiglitazone+Eno1 treatment group compared to rosiglitazone alone, indicating that Eno1 attenuates rosiglitazone induced weight gain.

FIG. 19 shows the effect of rosiglitazone and Eno1 on gained body weight in a diabetic mouse model (db/db mice). Treatment groups shown are Saline_Lean (saline treatment of lean mice); Saline-db (saline treatment of db/db mice); Rosi (rosiglitazone treatment of db/db mice, 20 mg/kg/day); and Rosi+Eno1 (combination of 20 mg/kg/day rosiglitazone and 400 μg/kg/day Eno1 treatment of db/db mice). Diabetic mice treated with rosiglitazone alone or rosiglitazone+Eno1 gained more body weight than control (saline treated) db/db mice. Body weight gain in Rosiglitazone treated mice was attenuated when mice were also administered Eno1.

FIG. 20 shows the effect of rosiglitazone and Eno1 on fed blood glucose levels in a diabetic mouse model (db/db mice). Treatment groups shown are Saline_Lean (saline treatment of lean mice); Saline-db (saline treatment of db/db mice); Rosi (rosiglitazone treatment of db/db mice, 20 mg/kg/day); and Rosi+Eno1 (combination of 20 mg/kg/day rosiglitazone and 400 μg/kg/day Eno1 treatment of db/db mice). The combination of rosiglitazone and Eno1 reduced blood glucose levels more quickly than rosiglitazone alone.

FIGS. 21A and 21B show fluorescent images of the tissue distribution in mice of (A) a fluorescently-labeled Eno1-G5-PAMAM dendrimer complex and (B) a fluorescently-labeled, muscle targeted Eno-1-G5-PAMAM dendrimer complex.

### DETAILED DESCRIPTION AND PREFERRED EMBODIMENTS

The present invention is based, at least in part, on the results of in vivo studies presented herein demonstrating a role for Eno1 muscle targeted fusion proteins in insulin dependent and independent glucose uptake, glucose tolerance, insulin sensitivity, and/or diabetes, e.g., type 1 diabetes, type 2 diabetes, pre-diabetes, and gestational diabetes. More specifically, administration of an Eno1 fusion protein comprising a muscle targeting peptide reduced fed blood glucose levels in a diabetic mouse model (db/db mice). Accordingly, compositions comprising Eno1 muscle targeted fusion proteins are provided. Further, variants of Eno1 fusion proteins in which certain serine residues are replaced with cysteine residues to provide reactive sites for attaching functional moieties, such as cell penetrating peptides or targeting groups (e.g., muscle targeting peptides, creatine or methoxypoly(ethylene glycol) (PEG)), are also provided. The results described herein demonstrate that muscle targeted Eno1 fusion proteins of the invention are effective in normalizing glucose, and thus indicate that these proteins are useful in improving glucose tolerance, to thereby treat glucose tolerance, insulin sensitivity, and/or diabetes.

### I. DEFINITIONS

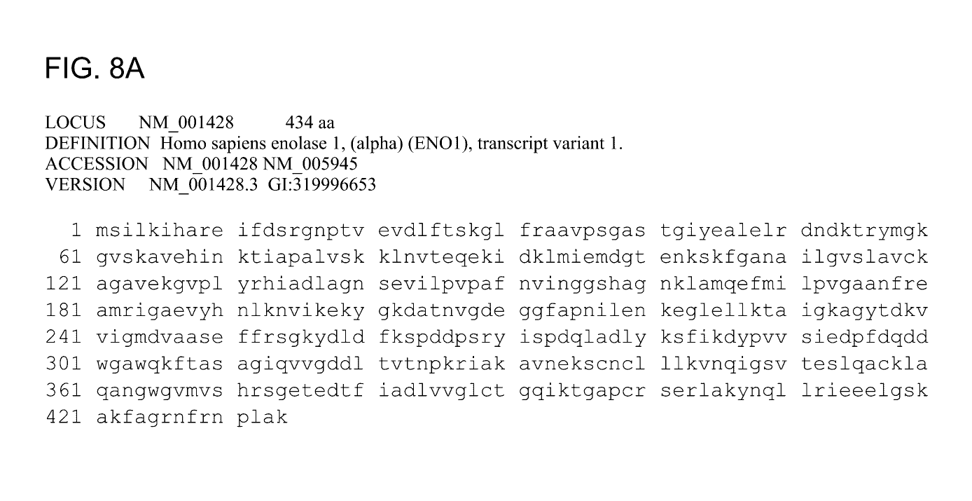

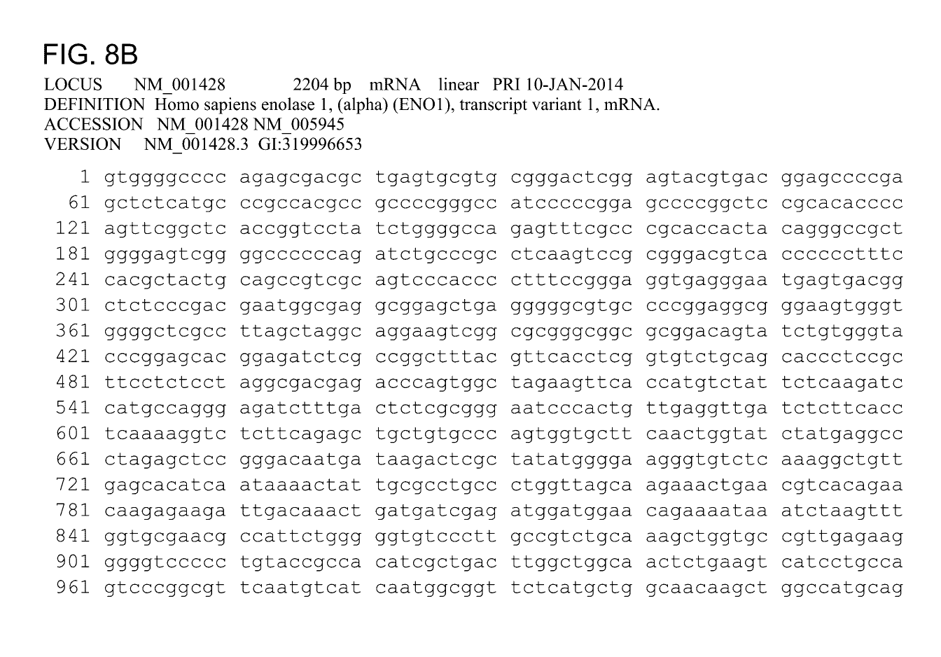

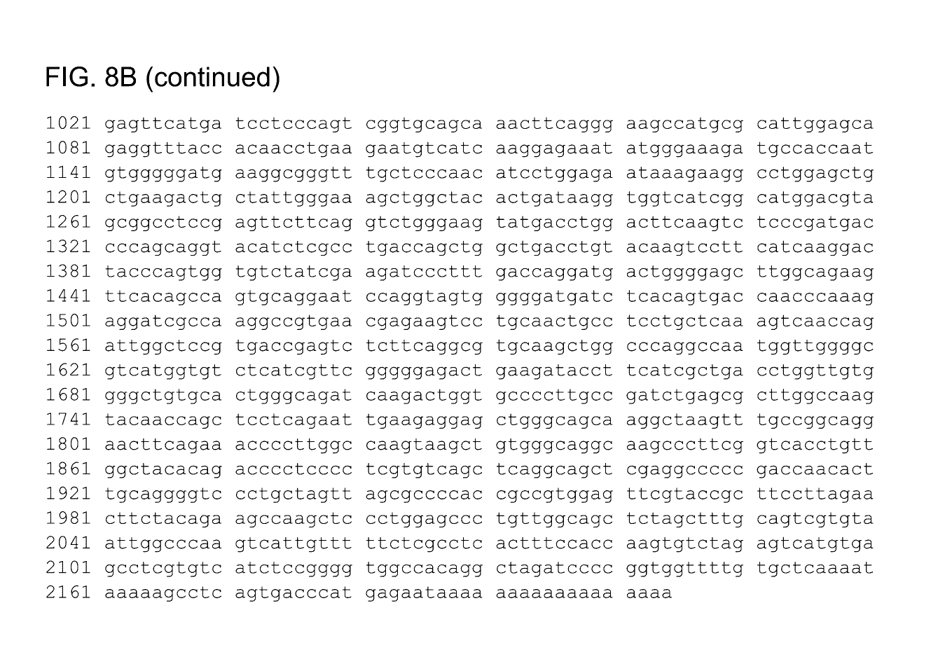

Enolase 1, (alpha), also known as ENO1L, alpha-enolase, enolase-alpha, tau-crystallin, non-neural enolase (NNE), alpha enolase like 1, phosphopyruvate hydratase (PPH), plasminogen-binding protein, MYC promoter-binding protein 1 (MPB1), and 2-phospho-D-glycerate hydro-lyase, is one of three enolase isoenzymes found in mammals. Protein and nucleic acid sequences of human Eno1 isoforms are provided herein in FIGS. 8-10. The instant application provides human amino acid and nucleic acid sequences for the treatment of human disease. However, it is understood that the compositions and methods of the invention can be readily adapted for treatment of non-human animals by selection of an Eno1 of the species to be treated Amino acid and nucleic acid sequences of Eno1 for non-human species are known in the art and can be found, for example, at ncbi.nlm.nih.gov/genbank/. In some embodiments, the Eno1 used in the compositions and methods of the invention is a mammalian Eno1. In a preferred embodiment, the Eno1 is human Eno1.

As used herein, an “Eno1 molecule” refers to a molecule comprising an Eno1 polypeptide or a fragment thereof. In certain embodiments, the Eno 1 molecule further comprises at least one functional moiety, such as a muscle targeting moiety, e.g., muscle targeting peptide, a cell penetrating peptide, a biocompatible polymer, or any combination thereof.

As used herein, “administration of Eno1” unless otherwise indicated is understood as administration of either Eno1 protein or a nucleic acid construct for expression of Eno1 protein. In certain embodiments the Eno1 protein can include an Eno1 protein fragment or a nucleic acid for encoding an Eno1 protein fragment. In certain embodiments, administration of Eno1 is administration of Eno1 protein. In certain embodiments, administration of Eno1 is administration of Eno1 polynucleotide. Protein and nucleic acid sequences of human Eno1 are provided herein. In certain embodiments, administration of Eno1 comprises administration of the first variant or the second variant of human Eno1. In certain embodiments, administration of Eno1 comprises administration of the first variant and the second variant of human Eno1. In certain embodiments, administration of Eno1 comprises administration of the first variant of human Eno1. In certain embodiments, administration of Eno1 comprises administration of the second variant of human Eno1. In certain embodiments, administration of Eno1 comprises administration of only the first variant of human Eno1. In certain embodiments, administration of Eno1 comprises administration of only the second variant of human Eno1.

As used herein, “biologically active” refers to an Eno1 molecule or fragment thereof that has at least one activity of an endogenous Eno1 protein. For example, in some embodiments, the biologically active Eno1 molecule or fragment thereof catalyzes the dehydration of 2-phospho-D-glycerate (PGA) to phosphoenolpyruvate (PEP). In some embodiments, the biologically active Eno1 molecule or fragment thereof catalyzes the hydration of PEP to PGA. In some embodiments, the biologically active Eno1 molecule or fragment thereof increases glucose uptake by a cell, for example a muscle cell, preferably a skeletal muscle cell. In some embodiments, the biologically active Eno1 molecule or fragment thereof reduces blood glucose levels, e.g. fed blood glucose levels or blood glucose levels in a glucose tolerance test. In some embodiments, the biologically active Eno1 molecule or fragment thereof binds to Nampt, for example, extracellular Nampt (eNampt).

As used herein, “administration to a muscle”, “delivery to a muscle”, or “delivery to a muscle cell” including a skeletal muscle cell, smooth muscle cell, and the like are understood as a formulation, method, or combination thereof to provide an effective dose of Eno1 to a muscle e.g., a muscle cell, to provide a desired systemic effect, e.g., normalization of blood glucose in a subject with abnormal blood glucose, e.g., by increasing glucose tolerance and/or insulin sensitivity, or treating diabetes. In certain embodiments, the Eno1 is formulated for administration directly to, and preferably retention in, muscle. In certain embodiments, the formulation used for administration directly to the muscle (i.e., intramuscular administration) preferably a sustained release formulation of the Eno1 to permit a relatively low frequency of administration (e.g., once per week or less, every other week or less, once a month or less, once every other month or less, once every three months or less, once every four months or less, once every five months or less, once every six months or less). In certain embodiments, the Eno1 is linked to a targeting moiety to increase delivery of the Eno1 to muscle so that the Eno1 need not be delivered directly to muscle (e.g., is delivered subcutaneously or intravenously). It is understood that administration to muscle does not require that the entire dose of Eno1 be delivered to the muscle or into muscle cells. In certain embodiments, at least 5%, at least 10%, at least 15%, at least 20%, at least 25%, at least 30%, at least 35% of the Eno1 is delivered to muscle, preferably skeletal muscle and/or smooth muscle. In certain embodiments, the amount of non-intramuscularly administered muscle-targeted Eno1 delivered to a muscle cell is about 1.5 or more times greater, 2 or more times greater, 3 or more times greater, 4 or more times greater, 5 or more times greater, or 6 or more times greater than the amount of non-targeted Eno1 delivered to muscle. In certain embodiments, the Eno1 is delivered to skeletal muscle. In certain embodiments, the Eno1 is delivered to smooth muscle. In certain embodiments, the Eno1 is delivered to skeletal muscle and smooth muscle. In certain embodiments, is delivered preferentially or in greater amount to skeletal muscle as compared to smooth muscle. In certain embodiments, at least 50%, 60%, 70%, 75%, 80%, 85%, 90%, 95% or greater of the Eno1 delivered to muscle is delivered to skeletal muscle. In certain embodiments, the Eno1 is not delivered to smooth muscle. Assays to determine the relative targeting of a payload by a targeting moiety are known in the art and provided, for example, in Samoylova et al., 1999, Muscle Nerve, 22:460-466, incorporated herein by reference.

As used herein, a “muscle targeting moiety” includes a muscle targeting peptide (MTP), for example a skeletal and/or smooth muscle targeting peptide (SMTP). In certain embodiments, the targeting moiety include ligands to bind integrins αvβ5 or αvβ3 integrins. In certain embodiments, the targeting moiety includes a CD-46 ligand. In certain embodiments, the targeting moiety includes an adenovirus peton protein optionally in combination with an adenovirus 35 fiber protein. In certain embodiments, at least 5%, at least 10%, at least 15%, at least 20%, at least 25%, at least 30%, at least 35% of muscle-targeted Eno1 is delivered to muscle, in some embodiments preferably skeletal and/or smooth muscle, by a muscle-targeting moiety. In certain embodiments, the amount of non-intramuscularly administered muscle-targeted Eno1 delivered to a muscle cell is about 1.1, 1.2, 1.3, 1.4, 1.5, 1.7, 1.8, 1.9 or more times greater, 2 or more times greater, 3 or more times greater, 4 or more times greater, 5 or more times greater, or 6 or more times greater than the amount of non-targeted Eno1 delivered to muscle. In certain embodiments, the amount of non-intramuscularly administered muscle-targeted Eno1 delivered to a muscle cell is increased by 5%, 10%, 15%, 20%, 25%, 30%, 35%, 40%, 45%, 50%, 55%, 60%, 65%, 70%, 75%, 80%, 90%, 100%, 150%, 200%, 300%, 400%, 500%, 600% or more as compared to the amount of non-targeted Eno1 delivered to muscle.

As used herein, a “muscle targeting peptide” or “MTP” is understood as a peptide sequence that increases the delivery of its payload (e.g., Eno1) to a muscle cell, preferably a skeletal and/or smooth muscle cell. MTPs are known in the art and are provided, for example, in U.S. Pat. No. 6,329,501; US Patent Publication No. 20110130346; and Samoylova et al., 1999, Muscle and Nerve 22: 460-466, each of which is incorporated herein in its entirety. In certain embodiments the MTP is a skeletal muscle targeting peptide. A “skeletal muscle targeting peptide” is a peptide sequence that increases the delivery of its payload (e.g., Eno1) to a skeletal muscle cell. In certain embodiments the MTP is a smooth muscle targeting peptide. A “smooth muscle targeting peptide” is a peptide sequence that increases the delivery of its payload (e.g., Eno1) to a smooth muscle cell. In certain embodiments the MTP increases the delivery of its payload (e.g., Eno1) to a skeletal cell and to a smooth muscle cell. In certain embodiments the MTP, e.g., skeletal muscle targeting peptide and/or smooth muscle targeting peptide, does not increase the delivery of its payload to cardiac muscle cell. MTP, e.g., skeletal muscle, targeting peptides include, but are not limited to peptides comprising the following sequences: ASSLNIA (SEQ ID NO: 7); WDANGKT (SEQ ID NO: 8); GETRAPL (SEQ ID NO: 9); CGHHPVYAC (SEQ ID NO: 10); and HAIYPRH (SEQ ID NO: 11).

In a preferred embodiment, the MTP comprises the amino acid sequence ASSLNIA (SEQ ID NO: 7).

As used herein, a “fusion protein” refers to a genetically engineered protein arising as a result of a laboratory induced mutation to a protein or polypeptide. For example, in some embodiments, a fusion protein comprises at least two peptides that are not found together in the same polypeptide in nature.

As used herein, an “ENO1 muscle targeted fusion protein” refers to a fusion protein comprising an ENO1 polypeptide, or a fragment thereof, and a muscle targeting moiety, e.g. a muscle targeting peptide.

As used herein, “payload” is understood as a moiety for delivery to a target cell by a targeting moiety. In certain embodiments, the payload is a peptide, e.g., an Eno1 peptide. In certain embodiments, the payload is a nucleic acid, e.g., a nucleic acid encoding an Eno1 peptide. In certain embodiments, the payload further comprises additional components (e.g., dendrimers, liposomes, microparticles) or agents (e.g., therapeutic agents) for delivery with the Eno1 payload to the target cell.

As used herein, an “added cysteine residue” is a cysteine residue that does not naturally occur in a native Eno1 protein. For example, in some embodiments, an added cysteine residue is a cysteine residue that is used to replace another amino acid residue (for example a serine residue or a threonine residue) in a native Eno1 polypeptide. In other embodiments, the added cysteine residue is a cysteine residue that is inserted into a native Eno1 polypeptide without replacing any of the amino acid residues of the native polypeptide. In a particular embodiment, the added cysteine residue is added to the N-terminus or the C-terminus of the Eno1 polypeptide.

As used herein, a “linker” is understood as a moiety that juxtaposes a functional moiety (e.g. a targeting moiety or cell penetrating peptide) and an Eno1 polypeptide or fragment thereof in sufficiently close proximity such that the functional moiety functions in its intended manner (e.g. the targeting moiety delivers Eno1 to the desired site or the cell penetrating peptide enhances cell penetration). In certain embodiments, the linker is a covalent linker, e.g., a cross-linking agent including a reversible cross-linking agent; or a peptide bond, e.g., wherein the payload is a protein co-translated with the targeting moiety. In certain embodiments, the linker is covalently joined to the Eno1 or the functional moiety and non-covalently linked to the other. In certain embodiments, the linker is a liposome or a microparticle, and the targeting moiety is exposed on the surface of the liposome and the payload, e.g., Eno1 is encapsulated in the liposome or microparticle. In certain embodiments, the linker and the Eno1 are present on the surface of the microparticle linker. In certain embodiments, the targeting moiety is present on the surface of a virus particle and the payload comprises a nucleic acid encoding Eno1.

As used herein, “linked”, “operably linked”, “joined” and the like refer to a juxtaposition wherein the components described are present in a complex permitting them to function in their intended manner. The components can be linked covalently (e.g., peptide bond, disulfide bond, non-natural chemical linkage), through hydrogen bonding (e.g. knob-into-holes pairing of proteins, see, e.g., U.S. Pat. No. 5,582,996; Watson-Crick nucleotide pairing), or ionic binding (e.g., chelator and metal) either directly or through linkers (e.g., peptide sequences, typically short peptide sequences; nucleic acid sequences; or chemical linkers, including the use of linkers for attachment to higher order or larger structures including microparticles, beads, or dendrimers). As used herein, components of a complex can be linked to each other wherein some of the components of the complex can be attached covalently and some non-covalently. Linkers can be used to provide separation between active molecules so that the activity of the molecules is not substantially inhibited (less than 10%, less than 20%, less than 30%, less than 40%, less than 50%) by linking the first molecule to the second molecule. Linkers can be used, for example, in joining Eno1 to a functional moiety (e.g. a targeting moiety, a cell penetrating peptide, or an added cysteine residue). As used herein, molecules that are linked, but no covalently joined, have a binding affinity (Kd) of less than 10⁻³, 10⁻⁴, 10⁻⁵, 10⁻⁶, 10⁻⁷, 10⁻⁸, 10⁻⁹, 10⁻¹⁰, 10⁻¹¹, or 10⁻¹², or any range bracketed by those values, for each other under conditions in which the reagents of the invention are used, i.e., typically physiological conditions.

In certain embodiments, the Eno1 and the functional moiety (e.g. a targeting moiety or a cell penetrating peptide) are present in a complex at about a 1:1 molar ratio. In certain embodiments, the functional moiety is present in a complex with a molar excess of Eno1. In certain embodiments, the ratio of the functional moiety to Eno1 is about 0.1:1, about 0.2:1, about 0.3:1, about 0.4:1, about 0.5:1, about 0.6:1, about 0.7:1, about 0.8:1, about 0.9:1, about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, or about 20:1.

As used herein a “biocompatible polymer” includes polyalkylene oxides such as without limitation polyethylene glycol (PEG), dextrans, colominic acids or other carbohydrate based polymers, polymers of amino acids, biotin derivatives, polyvinyl alcohol (PVA), polycarboxylates, polyvinylpyrrolidone, polyethylene-co-maleic acid anhydride, polystyrene-co-malic acid anhydride, polyoxazoline, polyacryloylmorpholine, heparin, albumin, celluloses, hydrolysates of chitosan, starches such as hydroxyethyl-starches and hydroxy propyl-starches, glycogen, agaroses and derivatives thereof, guar gum, pullulan, inulin, xanthan gum, carrageenan, pectin, alginic acid hydrolysates, other bio-polymers and any equivalents thereof. In a particular embodiment, the biocompatible polymer is polyethylene glycol. Other useful polyalkylene glycol compounds include polypropylene glycols (PPG), polybutylene glycols (PBG), PEG-glycidyl ethers (Epox-PEG), PEG-oxycarbonylimidazole (CDI-PEG), branched polyethylene glycols, linear polyethylene glycols, forked polyethylene glycols and multi-armed or “super branched” polyethylene glycols (star-PEG). Biocompatible polymers are described, for example, in U.S. Pat. No. 7,632,921, which is incorporated by reference herein in its entirety.

As used herein, “polyethylene glycol”, “PEG”, “PEG group”, or “mPEG” and the like refers to any water-soluble poly(ethylene oxide). PEG comprises polymer chains consisting of repeating polyethylene glycol units, also described as methoxypoly(ethylene glycol). The basic structure of PEG is:

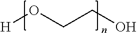

wherein n is the number of units in the polymer, and n ranges from 2 to 4000. Thus, PEGs for use in accordance with the invention may comprise the following structure “—(OCH₂CH₂)ₙ—” where (n) is 2 to 4000. As used herein, PEG also includes “—CH₂CH₂—O(CH₂CH₂O)ₙ—CH₂CH₂—” and “—(OCH₂CH₂)ₙO—,” depending upon whether or not the terminal oxygens have been displaced. The term “PEG” also includes structures having various terminal or “end capping” groups, such as without limitation a hydroxyl or a C₁₋₂₀ alkoxy group. The term “PEG” also means a polymer that contains a majority, that is to say, greater than 50%, of —OCH₂CH₂-repeating subunits. Each PEG group may be straight-chained (i.e., linear) or branched. When branched, a PEG polymer may be forked (Y-shaped), multi-arm (e.g., having more than one fork) or comb-shaped. In certain embodiments, PEG has a weight of between about 1 kDa and about 50 kDa.

As used herein, “pegylated” or “pegylation” and the like refer to covalently linking one or more polymer polyethylene glycol (PEG) groups to an Eno 1 protein as described above. PEG groups may be linked through reactive molecular groups on amino acid side chains, such as lysine, cysteine, histidine, arginine, aspartic acid, glutamic acid, serine, threonine, and/or tyrosine. In order for PEG groups to react with an amino acid, they must be functionalized with a reactive linker group such as a maleimide, vinyl sulfone, pyridyl disulfide, amine, carboxylic acid, or an N-hydroxysuccinimide (NHS) ester. Pegylation of a protein such as Eno 1 may lead to increased bioavailability, improved pharmacokinetics (e.g increased half-life of the protein), improved pharmacodynamics, less frequent administration, and/or lower immunogenicity.

As used herein, a “subject with elevated blood glucose” or “increased blood glucose” is understood as a subject who has elevated blood glucose for a sufficient duration and frequency to be considered a pathological condition, i.e., a subject that does not produce enough insulin or is not sufficiently sensitive to insulin so that the glucose level of the subject remains elevated for an extended period after eating a meal, e.g. for more than two hours after eating a meal and/or who has an elevated fasting blood glucose. In certain embodiments, a subject with elevated blood glucose is understood as a subject with one or both of fasting blood glucose of at least 100 mg/dl and 2-hour plasma glucose in a 75-g oral glucose tolerance test of at least 140 mg/dl. In certain embodiments, a subject with elevated blood glucose is understood as a subject with one or more of fasting blood glucose of at least 126 mg/dl; a 2-hour plasma glucose in a 75-g oral glucose tolerance test of at least 200 mg/dl; or a random plasma glucose of at least 200 mg/dl. In certain embodiments, a subject with elevated blood glucose is understood as a pregnant subject with one or more of fasting blood glucose of at least 92 mg/dl; a 1-hour plasma glucose in a 75-g oral glucose tolerance test of at least 180 mg/dl; and a 2-hour plasma glucose in a 75-g oral glucose tolerance test of at least 153 mg/dl. In certain embodiments as used herein, a subject with elevated blood glucose does not include subjects with type 1 diabetes or pancreatic disease that results in an absolute insulin deficiency. In certain embodiments as used herein, a subject with elevated blood glucose includes subjects with type 1 diabetes or pancreatic disease that results in an absolute insulin deficiency.

As used herein, a “subject with elevated HbA1c” or a “subject with elevated Alc” is understood as a subject with an HbA1c level of at least 5.7%. In certain embodiments, the subject has an HbA1c level of at least 6.5%.

As used herein, “diabetes” is intended to refer to either type 1 diabetes or type 2 diabetes, or both type 1 and type 2 diabetes, optionally in combination with gestational diabetes. In certain embodiments, diabetes includes type 2 diabetes. In certain embodiments, diabetes does not include type 1 diabetes. In certain embodiments, diabetes includes gestational diabetes. In certain embodiments, diabetes does not include gestational diabetes. In certain embodiments, diabetes includes pre-diabetes. In certain embodiments, diabetes does not include pre-diabetes. In certain embodiments, diabetes includes pre-diabetes, type 1 diabetes, and type 2 diabetes. In certain embodiments, diabetes includes pre-diabetes and type 2 diabetes.

As used herein, “insulin resistance” and “insulin insensitivity” can be used interchangeably and refers to conditions, especially pathological conditions, wherein the amount of insulin is less effective at lowering blood sugar than in a normal subject resulting in an increase in blood sugar above the normal range that is not due to the absence of insulin. Without being bound by mechanism, the conditions are typically associated with a decrease in signaling through the insulin receptor. Typically, insulin resistance in muscle and fat cells reduces glucose uptake and storage as glycogen and triglycerides, respectively. Insulin resistance in liver cells results in reduced glycogen synthesis and a failure to suppress glucose production and release into the blood.

Insulin resistance is often present in the same subject together with “insulin insufficiency”, which also results in an increase in blood sugar, especially a pathological increase in blood sugar, above the normal range that is not due to the absence of insulin. Insulin insufficiency is a condition related to a lack of insulin action in which insulin is present and produced by the body. It is distinct from type 1 diabetes in which insulin is not produced due to the lack of islet cells.

For the purposes of the methods of the instant invention, it is not necessary to distinguish if a subject suffers from insulin resistance/insensitivity, insulin insufficiency, or both.

The term “impaired glucose tolerance” (IGT) or “pre-diabetes” is used to describe a person who, when given a glucose tolerance test, has a blood glucose level that falls between normal and hyperglycemic, i.e., has abnormal glucose tolerance, e.g., pathologically abnormal glucose tolerance. Such a person is at a higher risk of developing diabetes although they are not clinically characterized as having diabetes. For example, impaired glucose tolerance refers to a condition in which a patient has a fasting blood glucose concentration or fasting serum glucose concentration greater than 110 mg/dl and less than 126 mg/dl (7.00 mmol/L), or a 2 hour postprandial blood glucose or serum glucose concentration greater than 140 mg/dl (7.78 mmol/L) and less than 200 mg/dl (11.11 mmol/L). Prediabetes, also referred to as impaired glucose tolerance or impaired fasting glucose is a major risk factor for the development of type 2 diabetes mellitus, cardiovascular disease and mortality. Much focus has been given to developing therapeutic interventions that prevent the development of type 2 diabetes by effectively treating prediabetes (Pharmacotherapy, 24:362-71, 2004).

As used herein, a “pathological” condition reaches a clinically acceptable threshold of disease or condition. A pathological condition can result in significant adverse effects to the subject, particularly in the long term, if the condition is not resolved, e.g., blood glucose and/or HbA1c levels are not normalized. Pathological conditions can be reversed by therapeutic agents, surgery, and/or lifestyle changes. A pathological condition may or may not be chronic. A pathological condition may or may not be reversible. A pathological condition may or may not be terminal.

“Hyperinsulinemia” is defined as the condition in which a subject with insulin resistance, with or without euglycemia, in which the fasting or postprandial serum or plasma insulin concentration is elevated above that of normal, lean individuals without insulin resistance (i.e., >100 mg/dl in a fasting plasma glucose test or >140 mg/dl in an oral glucose tolerance test).

The condition of “hyperglycemia” (high blood sugar) is a condition in which the blood glucose level is too high. Typically, hyperglycemia occurs when the blood glucose level rises above 180 mg/dl. Symptoms of hyperglycemia include frequent urination, excessive thirst and, over a longer time span, weight loss.

The condition of “hypoglycemia” (low blood sugar) is a condition in which the blood glucose level is too low. Typically, hypoglycemia occurs when the blood glucose level falls below 70 mg/dl. Symptoms of hypoglycemia include moodiness, numbness of the extremities (especially in the hands and arms), confusion, shakiness or dizziness. Since this condition arises when there is an excess of insulin over the amount of available glucose it is sometimes referred to as an insulin reaction.

As used herein, an “HbA1c level” or “A1c level” is understood as a hemoglobin Alc (HbA1c) level determined from an HbA1c test, which assesses the average blood glucose levels during the previous two and three months. A person without diabetes typically has an HbA1c value that ranges between 4% and 6%. Prediabetes is characterized by a pathological HbA1c level of 5.7% to 6.5%, with an Hb1Ac level greater than 6.5% being indicative of diabetes. Every 1% increase in HbA1c reflects a blood glucose levels increases by approximately 30 mg/dL and increased risk of complications due to persistent elevated blood glucose. Preferably, the HbA1c value of a patient being treated according to the present invention is reduced to less than 9%, less than 7%, less than 6%, and most preferably to around 5%. Thus, the excess HbA1c level of the patient being treated (i.e., the Hb1Ac level in excess of 5.7%) is preferably lowered by at least 10%, 20%, 30%, 40%, 50%, 60%, 70%, 80%, 90%, or more relative to such levels prior to treatment (i.e., pre-treatment level-post-treatment level/pre-treatment level).

As used herein, the term “therapeutic treatment that induces weight gain” refers to any method of drug for the treatment of a disorder that results in increased body mass in a subject. Increased body mass can be relative to a subject or population of subjects that does not receive the treatment, or relative to the body mass of subject or population of subjects prior to treatment. Therapeutic treatments that induce weight gain include, but are not limited to, therapeutic agents for the treatment of diabetes, antipsychotic agents, antidepressants, mood stabilizers, anticonvulsants, steroid hormones, prednisone beta-blockers, oral contraceptives, antihistamines, HIV antiretroviral drugs, antiseizure and antimigraine drugs, protease inhibitors, antihyperlipemic agents, hypotensive or antihypertensive agents, anti-obesity agents, diuretics, chemotherapeutic agents, immunotherapeutic agents, and immunosuppressive agents.

“Obesity” or “obese” refers to the condition where a patient has a body mass index (BMI) equal to or greater than 30 kg/m². “Visceral obesity” refers to a waist to hip ration of 1.0 in male patients and 0.8 in female patients. In another aspect, visceral obesity defines the risk for insulin resistance and the development of pre-diabetes.

“Overweight” or “subject afflicted with an overweight condition” refers to a patient with a body mass index (BMI) greater than or equal to 25 kg/m² and less than 30 kg/m². “Weight gain” refers to the increase in body weight in relationship to behavioral habits or addictions, e.g., overeating or gluttony, smoking cessation, or in relationship to biological (life) changes, e.g., weight gain associated with aging in men and menopause in women or weight gain after pregnancy, or as a side effect of a therapeutic treatment, e.g., a treatment known to induce or cause weight gain.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, the terms “treat,” “treating” or “treatment” refer, preferably, to an action to obtain a beneficial or desired clinical result including, but not limited to, alleviation or amelioration of one or more signs or symptoms of a disease or condition, diminishing the extent of disease, stability (i.e., not worsening) state of disease, amelioration or palliation of the disease state. As used herein, treatment can include one or more of reduction of insulin resistance, increasing insulin sensitivity, decreasing insulin deficiency, improving or normalizing HbAc1 levels, improving or normalizing blood glucose levels (e.g., fed blood glucose levels, fasting blood glucose levels, glucose tolerance), and ameliorating at least one sign or symptom of diabetes. Therapeutic goals in the treatment of diabetes, including type 2 diabetes, include HbAc1 levels <6.5%; blood glucose 80-120 mg/dl before meals; and blood glucose <140 mg/dl 2 hours after meals. Therapeutic goals in the treatment of pre-diabetes include reduction of HbA1c, blood glucose levels, and glucose response to normal levels. Treatment does not need to be curative or reach the ideal therapeutic goals of treatment. Treatment outcomes need not be determined quantitatively. However, in certain embodiments, treatment outcomes can be quantitated by considering percent improvement towards a normal value at the end of a range. For example, metabolic syndrome is characterized by an excess of some measures (e.g., blood glucose levels, HbA1c levels) and a deficiency in other measures (e.g., insulin response). A subject with a fasting blood glucose level of 150 mg/dl would have excess fasting blood glucose of 50 mg/dl (150 mg/dl-100 mg/dl, the maximum normal blood glucose level). Reduction of excess blood glucose by 20% would be an 10 mg/dl reduction in excess blood glucose. Similar calculations can be made for other values.

As used herein, “reducing glucose levels” means reducing excess of glucose by at least 10%, 20%, 30%, 40%, 50%, 60%, 70%, 80%, 90%, 95%, or more to achieve a normalized glucose level, i.e., a glucose level no greater than 150 mg/dl. Desirably, glucose levels prior to meals are reduced to normoglycemic levels, i.e., between 150 to 60 mg/dL, between 140 to 70 mg/dL, between 130 to 70 mg/dL, between 125 to 80 mg/dL, and preferably between 120 to 80 mg/dL. Such reduction in glucose levels may be obtained by increasing any one of the biological activities associated with the clearance of glucose from the blood. Accordingly, an agent having the ability to reduce glucose levels may increase insulin production, secretion, or action. Insulin action may be increased, for example, by increasing glucose uptake by peripheral tissues and/or by reducing hepatic glucose production. Alternatively, the agent may reduce the absorption of carbohydrates from the intestines, alter glucose transporter activity (e.g., by increasing GLUT4 expression, intrinsic activity, or translocation), increase the amount of insulin-sensitive tissue (e.g., by increasing muscle cell or adipocyte cell differentiation), or alter gene transcription in adipocytes or muscle cells (e.g., altered secretion of factors from adipocytes expression of metabolic pathway genes). Desirably, the agent increases more than one of the activities associated with the clearance of glucose.

By “alter insulin signaling pathway such that glucose levels are reduced” is meant to alter (by increasing or reducing) any one of the activities involved in insulin signaling such that the overall result is an increase in the clearance of glucose from plasma and normalizes blood glucose. For example, altering the insulin signaling pathway thereby causing an increase in insulin production, secretion, or action, an increasing glucose uptake by peripheral tissues, a reducing hepatic glucose production, or a reducing the absorption of carbohydrates from the intestines.

A “therapeutically effective amount” is that amount sufficient to treat a disease in a subject. A therapeutically effective amount can be administered in one or more administrations.

A number of treatments for type 2 diabetes are known in the art including both drug and behavioral interventions. Drugs for treatment of type 2 diabetes include, but are not limited to meglitinides (repaglinide (Prandin) and nateglinide (Starlix); sulfonylureas (glipizide (Glucotrol), glimepiride (Amaryl), and glyburide (DiaBeta, Glynase)); Dipeptidy peptidase-4 (DPP-4) inhibitors (saxagliptin (Onglyza), sitagliptin (Januvia), and linagliptin (Tradjenta)); biguanides (metformin (Fortamet, Glucophage)); thiazolidinediones (rosiglitazone (Avandia) and pioglitazone (Actos)); and alpha-glucosidase inhibitors (acarbose (Precose) and miglitol (Glyset)). Insulins are typically used only in treatment of later stage type 2 diabetes and include rapid-acting insulin (insulin aspart (NovoLog), insulin glulisine (Apidra), and insulin lispro (Humalog)); short-acting insulin (insulin regular (Humulin R, Novolin R)); intermediate-acting insulin (insulin NPH human (Humulin N, Novolin N)), and long-acting insulin (insulin glargine (Lantus) and insulin detemir (Levemir)). Treatments for diabetes can also include behavior modification including exercise and weight loss which can be facilitated by the use of drugs or surgery. Treatments for elevated blood glucose and diabetes can be combined. For example, drug therapy can be combined with behavior modification therapy.

The terms “administer”, “administering” or “administration” include any method of delivery of a pharmaceutical composition or agent into a subject's system or to a particular region in or on a subject. In certain embodiments, the agent is administered enterally or parenterally. In certain embodiments of the invention, an agent is administered intravenously, intramuscularly, subcutaneously, intradermally, intranasally, orally, transcutaneously, or mucosally. In certain preferred embodiments, an agent is administered by injection or infusion, e.g., intravenously, intramuscularly, subcutaneously. In certain embodiments, administration includes the use of a pump. In certain embodiments, the agent is administered locally or systemically. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, etc.

As used herein, the term “co-administering” refers to administration of Eno1 prior to, concurrently or substantially concurrently with, subsequently to, or intermittently with the administration of an agent for the treatment of diabetes, pre-diabetes, glucose intolerance, or insulin resistance. The Eno1 formulations provided herein, can be used in combination therapy with at least one other therapeutic agent for the treatment of diabetes, pre-diabetes, glucose intolerance, or insulin resistance. Eno1 and/or pharmaceutical formulations thereof and the other therapeutic agent can act additively or, more preferably, synergistically. In one embodiment, Eno1 and/or a formulation thereof is administered concurrently with the administration of another therapeutic agent for the treatment of diabetes, pre-diabetes, glucose intolerance, or insulin resistance. In another embodiment, Eno1 and/or a pharmaceutical formulation thereof is administered prior or subsequent to administration of another therapeutic agent for the treatment of diabetes, pre-diabetes, glucose intolerance, or insulin resistance.

The term “sample” as used herein refers to a collection of similar fluids, cells, or tissues isolated from a subject. The term “sample” includes any body fluid (e.g., urine, serum, blood fluids, lymph, gynecological fluids, cystic fluid, ascetic fluid, ocular fluids, and fluids collected by bronchial lavage and/or peritoneal rinsing), ascites, tissue samples or a cell from a subject. Other subject samples include tear drops, serum, cerebrospinal fluid, feces, sputum, and cell extracts. In a particular embodiment, the sample is urine or serum. In certain embodiments, the sample comprises cells. In other embodiments, the sample does not comprise cells.

The term “control sample,” as used herein, refers to any clinically relevant comparative sample, including, for example, a sample from a healthy subject not afflicted with any of impaired glucose tolerance, increased blood glucose, insulin resistance, diabetes, or prediabetes; or a sample from a subject from an earlier time point in the subject, e.g., prior to treatment, at an earlier stage of treatment. A control sample can be a purified sample, protein, and/or nucleic acid provided with a kit. Such control samples can be diluted, for example, in a dilution series to allow for quantitative measurement of analytes in test samples. A control sample may include a sample derived from one or more subjects. A control sample may also be a sample made at an earlier time point from the subject to be assessed. For example, the control sample can be a sample taken from the subject to be assessed before the onset abnormal blood glucose levels or A1c levels, at an earlier stage of disease, or before the administration of treatment or of a portion of treatment. The control sample may also be a sample from an animal model, or from a tissue or cell lines derived from the animal model of impaired glucose tolerance, increased blood glucose, insulin resistance, diabetes, or prediabetes. The level of Eno1 activity or expression in a control sample that consists of a group of measurements may be determined, e.g., based on any appropriate statistical measure, such as, for example, measures of central tendency including average, median, or modal values.

The term “control level” refers to an accepted or pre-determined level of a sign of a impaired glucose tolerance, increased blood glucose, insulin resistance, diabetes, or pre-diabetes in a subject or a subject sample. The following levels are considered to be normal levels: (i) fasting blood glucose less than or equal to 100 mg/dl; (ii) HbA1c less than or equal to 5.7%; (iii) oral glucose tolerance test less than or equal to 140 mg/dl. Levels above these levels are understood to be pathological levels.

The terms “modulate” or “modulation” refer to upregulation (i.e., activation or stimulation), downregulation (i.e., inhibition or suppression) of a level, or the two in combination or apart. A “modulator” is a compound or molecule that modulates, and may be, e.g., an agonist, antagonist, activator, stimulator, suppressor, or inhibitor.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, or protein, or both.

The terms “level of expression of a gene” or “gene expression level” refer to the level of mRNA, as well as pre-mRNA nascent transcript(s), transcript processing intermediates, mature mRNA(s) and degradation products, or the level of protein, encoded by the gene in the cell.

As used herein, the term “antigen” refers to a molecule, e.g., a peptide, polypeptide, protein, fragment, or other biological moiety, which elicits an antibody response in a subject, or is recognized and bound by an antibody.

As used herein, the term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The articles “a”, “an” and “the” are used herein to refer to one or to more than one (i.e. to at least one) of the grammatical object of the article unless otherwise clearly indicated by contrast. By way of example, “an element” means one element or more than one element.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The recitation of a listing of chemical group(s) in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, modifications, and equivalents as may be included within the spirit and scope of the invention as defined by the appended claims.

### II. ENOLASE 1

Enolase 1, (alpha), also known as ENO1L, alpha-enolase, enolase-alpha, tau-crystallin, non-neural enolase (NNE), alpha enolase like 1, phosphopyruvate hydratase (PPH), plasminogen-binding protein, MYC promoter-binding protein 1 (MPB1), and 2-phospho-D-glycerate hydro-lyase, is one of three enolase isoenzymes found in mammals. Each isoenzyme is a homodimer composed of 2 alpha, 2 gamma, or 2 beta subunits, and functions as a glycolytic enzyme. Alpha-enolase in addition, functions as a structural lens protein (tau-crystallin) in the monomeric form. Alternative splicing of this gene results in a shorter isoform that has been shown to bind to the c-myc promoter and function as a tumor suppressor. Several pseudogenes have been identified, including one on the long arm of chromosome 1. Alpha-enolase has also been identified as an autoantigen in Hashimoto encephalopathy. Further information regarding human Eno1 can be found, for example, in the NCBI gene database under Gene ID No. 2023 (see, www.ncbi.nlm.nih.gov/gene/2023, incorporated herein by reference in the version available on the date of filing this application).

### Eno1 Variants

Two isoforms of human Eno1 are known. Protein and mRNA sequences of Homo sapiens enolase 1, (alpha) (ENO1), transcript variant 1, mRNA can be found at GenBank Accession No. NM_001428 (see www.ncbi.nlm.nih.gov/nuccore/NM_001428.3, which is incorporated by reference in the version available on the date of filing the instant application). This variant encodes the longer isoform, which is localized to the cytosol, and has alpha-enolase activity. It has been reported that the monomeric form of this isoform functions as a structural lens protein (tau-crystallin), and the dimeric form as an enolase. In a preferred embodiment of the invention, Eno1 is the transcript variant 1 of Eno1.

Protein and mRNA sequences of the Homo sapiens enolase 1, (alpha) (ENO1), transcript variant 2, mRNA can be found at GenBank Accession No. NM_001201483 (see www.ncbi.nlm.nih.gov/nuccore/NM_001201483.1, which is incorporated by reference in the version available on the date of filing the instant application). This variant differs at the 5′ end compared to variant 1, and initiates translation from an in-frame downstream start codon, resulting in a shorter isoform (MBP-1). This isoform is localized to the nucleus, and functions as a transcriptional repressor of c-myc protooncogene by binding to its promoter. In certain embodiments of the invention, Eno1 is the transcript variant 2 of Eno1.

Several additional variants of the Eno1 protein have been described, for example, in the UniProtKB/Swiss-Prot database under Accession No. P06733. Examples of Eno1 protein variants are shown in Table 1 below.

TABLE 1
Eno1 variants.

| | AA | |
|---|---|---|
| | residue | Modification |
| AA modification | 2 | N-acetylserine |
| AA modification | 5 | N6-acetyllysine |
| AA modification | 44 | Phosphotyrosine |
| AA modification | 60 | N6-acetyllysine; alternate |
| AA modification | 60 | N6-succinyllysine; alternate |
| AA modification | 64 | N6-acetyllysine |
| AA modification | 71 | N6-acetyllysine |
| AA modification | 89 | N6-acetyllysine; alternate |
| AA modification | 89 | N6-succinyllysine; alternate |
| AA modification | 92 | N6-acetyllysine |
| AA modification | 126 | N6-acetyllysine |
| AA modification | 193 | N6-acetyllysine |
| AA modification | 199 | N6-acetyllysine |
| AA modification | 202 | N6-acetyllysine |
| AA modification | 228 | N6-acetyllysine; alternate |
| AA modification | 228 | N6-succinyllysine; alternate |
| AA modification | 233 | N6-acetyllysine; alternate |
| AA modification | 233 | N6-malonyllysine; alternate |
| AA modification | 254 | Phosphoserine |
| AA modification | 256 | N6-acetyllysine |
| AA modification | 263 | Phosphoserine |
| AA modification | 272 | Phosphoserine |
| AA modification | 281 | N6-acetyllysine |
| AA modification | 285 | N6-acetyllysine |
| AA modification | 287 | Phosphotyrosine |
| AA modification | 335 | N6-acetyllysine |
| AA modification | 343 | N6-acetyllysine |
| AA modification | 406 | N6-acetyllysine |
| AA modification | 420 | N6-acetyllysine; alternate |
| AA modification | 420 | N6-malonyllysine; alternate |
| AA modification | 420 | N6-succinyllysine; alternate |
| Natural variant | 177 | N → K. |
| | | Corresponds to variant |
| | | rs11544513 [dbSNP \| |
| | | Ensembl]. |
| Natural variant | 325 | P → Q. |
| | | Corresponds to variant |
| | | rs11544514 [dbSNP \| |
| | | Ensembl]. |
| Mutagenesis | 94 | M → I: MBP1 protein production. No |
| | | MBP1 protein production; when |
| | | associated with I-97. |
| Mutagenesis | 97 | M → I: MBP1 protein production. No |
| | | MBP1 protein production; when |
| | | associated with I-94. |
| Mutagenesis | 159 | Dramatically decreases activity levels |
| Mutagenesis | 168 | Dramatically decreases activity levels |
| Mutagenesis | 211 | Dramatically decreases activity levels |
| Mutagenesis | 345 | Dramatically decreases activity levels |
| Mutagenesis | 384 | L → A: Loss of transcriptional repression |
| | | and cell growth inhibition; when |
| | | associated with A-388. |
| Mutagenesis | 388 | L → A: Loss of transcriptional repression |
| | | and cell growth inhibition; when |
| | | associated with A-384. |
| Mutagenesis | 396 | Dramatically decreases activity levels |

In certain embodiments of the invention, Eno1 is one of the variants listed in Table 1.

In some embodiments, the Eno1 comprises a nucleic acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the nucleic acid sequence of SEQ ID NO: 1 or SEQ ID NO: 3.

In some embodiments, the Eno1 consists of a nucleic acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the nucleic acid sequence of SEQ ID NO: 1 or SEQ ID NO: 3.

In some embodiments, the Eno1 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the amino acid sequence of SEQ ID NO: 2, SEQ ID NO: 4 or SEQ ID NO: 5. In a particular embodiment, the Eno1 comprises the amino acid sequence of SEQ ID NO: 5.

In some embodiments, the Eno1 consists of an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the amino acid sequence of SEQ ID NO: 2, SEQ ID NO: 4 or SEQ ID NO: 5. In a particular embodiment, the Eno1 consists of the amino acid sequence of SEQ ID NO: 5.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percentage sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol. 147(1); 195-7).

The term “hybridization” as defined herein is a process wherein substantially homologous complementary nucleotide sequences anneal to each other. The term “stringency” refers to the conditions under which a hybridization takes place. The stringency of hybridization is influenced by conditions such as temperature, salt concentration, ionic strength and hybridization buffer composition. Generally, low stringency conditions are selected to be about 30° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. High stringency hybridization conditions are typically used for isolating hybridizing sequences that have high sequence similarity to the target nucleic acid sequence. However, nucleic acids may deviate in sequence and still encode a substantially identical polypeptide, due to the degeneracy of the genetic code. Therefore medium stringency hybridization conditions may sometimes be needed to identify such nucleic acid molecules.

For example, typical high stringency hybridization conditions for DNA hybrids longer than 50 nucleotides encompass hybridization at 65° C. in 1×SSC or at 42° C. in 1×SSC and 50% formamide, followed by washing at 65° C. in 0.3×SSC. Examples of medium stringency hybridization conditions for DNA hybrids longer than 50 nucleotides encompass hybridization at 50° C. in 4×SSC or at 40° C. in 6×SSC and 50% formamide, followed by washing at 50° C. in 2×SSC. 1×SSC is 0.15M NaCl and 15 mM sodium citrate; the hybridization solution and wash solutions may additionally include 5×Denhardt's reagent, 0.5-1.0% SDS, 100 μg/ml denatured, fragmented salmon sperm DNA, 0.5% sodium pyrophosphate. In a preferred embodiment high stringency conditions mean hybridization at 65° C. in 0.1×SSC comprising 0.1% SDS and optionally 5×Denhardt's reagent, 100 μg/ml denatured, fragmented salmon sperm DNA, 0.5% sodium pyrophosphate, followed by the washing at 65° C. in 0.3×SSC. For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

In some embodiments, the Eno1 hybridizes to the complement of the nucleic acid sequence of SEQ ID NO: 1 or SEQ ID NO: 3 under high stringency hybridization conditions or medium stringency hybridization conditions as defined above.

In certain embodiments, the fragment of the Eno1 polypeptide comprises at least 10, 20, 30, 40, 50, 60, 70, 80, 90, 100, 150, 200, 250, 300, 350 or 400 amino acid residues.

### Eno1 Comprising Added Cysteine Residues

In some embodiments, the Eno1 comprises at least one or more added cysteine residues. The added cysteine residue provides a reactive site that enables defined chemistry, for example for attaching functional moieties such as a cell penetrating peptide or muscle targeting moiety. In some embodiments, the added cysteine residue replaces a residue in the native Eno1 polypeptide or a fragment thereof, for example, a serine residue or a threonine residue.

Selection of the amino acid residues for substitution may be based on the crystal structure of human Eno1 (e.g. PDB ID: 3B97; available at ncbi.nlm.nih.gov/Structure/mmdb/mmdbsrv.cgi?uid=66725). In certain embodiments, serine or threonine residues may be selected for substitution with an added cysteine, since their structures are similar to cysteine and less likely to disrupt protein structure and function. In some embodiments, serine and threonine residues with 100% solvent exposed R chains may be selected. Residues in active enzyme cleft locations may be avoided to prevent disruption of enzyme activity. Seven serine residues were identified with the above characteristics: S26, S78, S140, S253, S267, S236 and S418 (numbering is based on the human Eno1 sequence with the N-terminal methionine removed, SEQ ID NO: 13).

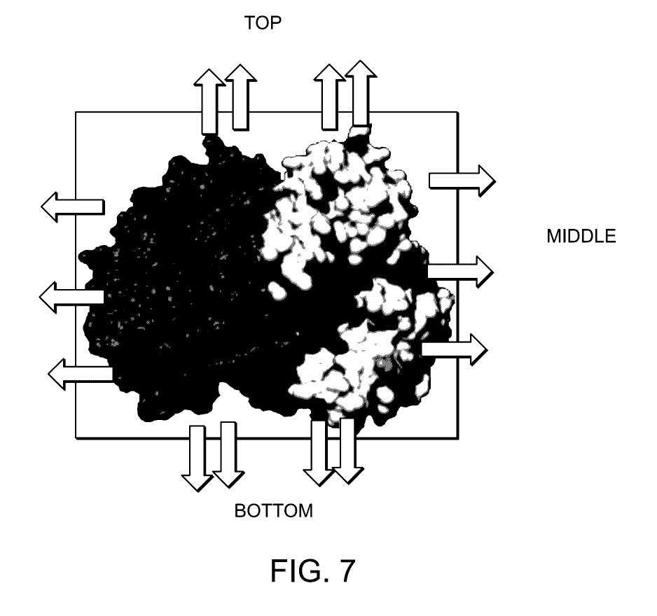

Orientation of the three dimensional model of the Eno1 dimer along the axis of symmetry with the N-terminus at the top (see FIG. 7) reveals that positions S26 and S78 are at the top of the dimer, S140 and S418 are at the side of the dimer (near C-terminus) and S236, S253 and S267 are at the bottom. In addition, the crystal structure reveals that sites near the N-terminus (i.e. the top of the dimer) point in the same direction (up), sites at the middle point in opposite directions, and sites at the bottom point in the same direction (down). See FIG. 7. In some cases, it may be optimal to have all of the functional peptides situated facing in the same direction to capture cooperative (avidity) effects. However, in other cases closely situated peptides may self assemble and become inactive. In addition, for some peptides such as cell penetrating peptides or targeting peptides, it may be beneficial to attach several peptides to the dimer to improve cell penetration or targeting.

In a particular embodiment, an added cysteine residue replaces one or more of the serine residues at positions 26, 78, 140, 236, 253, 267, or 418 of the native human Eno1 protein, transcript variant 1, with the N-terminal methionine removed (SEQ ID NO: 13). Any combination and number of substitutions at the serine positions described above may be made. In particular embodiments, an added cysteine residue replaces a serine residue at positions 26 and 78; positions 26, 418 and 267; positions 140, 418 and 267; positions 236, 253 and 267; positions 140 and 418; or positions 236, 253 and 267 of SEQ ID NO: 13. In a further particular embodiment, an added cysteine residue replaces the serine residue at position 267 of SEQ ID NO: 13.

In certain embodiments, an added cysteine residue replaces two of the serine residues at amino acid positions selected from positions 26, 78, 140, 236, 253, 267, or 418 of the native human Eno1 protein, transcript variant 1, with the N-terminal methionine removed (SEQ ID NO: 13), for example, at positions 26 and 78, 26 and 140, 26 and 236, 26 and 253, 26 and 267, 26 and 418, 78 and 140, 78 and 236, 78 and 253, 78 and 267, 78 and 418, 140 and 236, 140 and 253, 140 and 267, 140 and 418, 236 and 253, 236 and 267, 236 and 418, 253 and 267, 253 and 418, or 267 and 418.

In certain embodiments, an added cysteine residue replaces three of the serine residues at amino acid positions selected from 26, 78, 140, 236, 253, 267, or 418 of the native human Eno1 protein, transcript variant 1, with the N-terminal methionine removed (SEQ ID NO: 13), for example, at positions 26, 78 and 140; 26, 78 and 236; 26, 78 and 253; 26, 78 and 267; 26, 78 and 418; 26, 140 and 236; 26, 140 and 253; 26, 140 and 267; 26, 140 and 418; 26, 236 and 253; 26, 236 and 267; 26, 236 and 418; 26, 253 and 267; 26, 267 and 418; 78, 140 and 236; 78, 140 and 253; 78, 140 and 267; 78, 140 and 418; 78, 236 and 253; 78, 236 and 267; 78, 236 and 418; 78, 253 and 267; 78, 253 and 418; 140, 236 and 253; 140, 236 and 267; 140, 236 and 418; 140, 253 and 267; 140, 253 and 419; 140, 267 and 419; 236, 253 and 267; 236, 253 and 418; 236, 267 and 418; or 253, 267 and 418.

In other embodiments, amino acid residues of Eno1 that are partially solvent exposed (i e amino acid residues that are not 100% solvent exposed) may be selected for substitution with cysteine. Examples of serine and threonine residues of Eno1 that are partially solvent exposed include T40, S62, T71, T99, S103, and S309 (numbering is based on the human Eno1 sequence with the N-terminal methionine removed, SEQ ID NO: 13). In some embodiments, amino acid residues of Eno1 that are greater than 50%, 60%, 70%, 80% or 90% solvent exposed may be selected for substitution with cysteine. In some embodiments, amino acid residues of Eno1 other than serine and threonine may be selected for substitution with cysteine. Examples of amino acid residues of Eno1 other than serine and threonine that are greater than 90% solvent exposed and that may be selected for substitution with cysteine include N51, K53, K80, E95, A175, E197, D237, P263, K174, A308, N332, A361, E415, K419, L431, A432, and K433 (numbering is based on the human Eno1 sequence with the N-terminal methionine removed, SEQ ID NO: 13). Any of these substitutions may be combined with substitutions of serine residues at positions 26, 78, 140, 236, 253, 267, or 418 of the native human Eno1 protein, transcript variant 1, with the N-terminal methionine removed (SEQ ID NO: 13), as described above.

It will be understood that the present invention is intended to encompass ENO 1 fusion proteins in which any of the substitutions described above may be made in any ENO variant, e.g., the variants listed in Table 1, by replacing the corresponding amino acids in that particular variant. A person of ordinary skill in the art would be able to determine the amino acid positions for replacement by routine methods, for example by aligning the amino acid sequence of SEQ ID NO: 13 with the amino acid sequence of the variants described in Table 1 and identifying the corresponding amino acid position in the variant polypeptide.

In other embodiments, the added cysteine residue is attached to the N-terminus or the C-terminus of the Eno1 polypeptide or fragment thereof. In some embodiments, the added cysteine residue is attached to the Eno1 polypeptide or fragment thereof via a linker. In a particular embodiment, the linker comprises the amino acid sequence of SEQ ID NO: 14. In a further particular embodiment, the linker consists of the amino acids sequence of SEQ ID NO: 14. In some embodiments, the Eno1 polypeptide or fragment thereof comprises 1, 2, 3, 4, 5, 6, 7, 8, 9 or 10 added cysteine residues. In some embodiments, the Eno1 polypeptide or fragment thereof comprises at least 1, 2, 3, 4, 5, 6, 7, 8, 9 or 10 added cysteine residues.

In some aspects, the invention also relates to a nucleic acid sequence encoding any of the Eno1 polypeptides or fragments thereof comprising one or more added cysteine residues as described above.

### Biocompatible Polymers

The biocompatible polymer used for conjugation to Eno1 molecules, e.g., Eno1 fusion proteins, may be any of the polymers discussed above. The biocompatible polymer may be selected to provide desired improvements in pharmacokinetics (e.g. increased half-life of the Eno1 protein) or reduced immunogenicity. For example, in some embodiments, the identity, size and structure of the polymer is selected to improve the circulation half-life of the Eno1 protein or decrease the antigenicity of the polypeptide without an unacceptable decrease in activity. In particular embodiments, the polymer comprises PEG. In a further particular embodiment, the biocompatible polymer has at least 50% of its molecular weight as PEG. In one embodiment, the polymer is a polyethylene glycol terminally capped with an end-capping moiety such as hydroxyl, alkoxy, substituted alkoxy, alkenoxy, substituted alkenoxy, alkynoxy, substituted alkynoxy, aryloxy and substituted aryloxy. In a particular embodiment, the biocompatible polymer comprises methoxypolyethylene glycol. In a further particular embodiment, the biocompatible polymer comprises a methoxypolyethylene glycol having a size range from 1 kD to 50 kD, 3 kD to 100 kD, from 5 kD to 64 kD or from 5 kD to 43 kD.

In certain embodiments, the polymer has a reactive moiety. For example, in one embodiment, the polymer has a sulfhydryl reactive moiety that can react with a free cysteine on a functional Eno1 polypeptide to form a covalent linkage. Such sulfhydryl reactive moieties include thiol, triflate, tresylate, aziridine, oxirane, S-pyridyl or maleimide moieties. In a particular embodiment, the reactive moiety is a maleimide moiety. In one embodiment, the polymer is linear and has a “cap” at one terminus that is not strongly reactive towards sulfhydryls (such as methoxy) and a sulfhydryl reactive moiety at the other terminus. In a particular embodiment, the biocompatible polymer comprises PEG-maleimide and has a size range from 1 kD to 50 kD.

In certain embodiments, the biocompatible polymer is PEG. Pegylation is a technique of conjugating polymer polyethylene glycol (PEG) groups onto a protein, or fragment thereof. The resulting macromolecule typically has significantly altered physicochemical characteristics, such as increased half-life, increased solubility, increased drug stability, lower toxicity, and/or low immunogenicity. Additional advantages include reduced dosing frequency, which can lead to greater patient compliance and therefore, therapeutic efficacy.

In certain embodiments, the conjugates of the Eno1 molecule, e.g., fusion protein, and the biocompatible polymer are prepared by first replacing the codon for one or more amino acids on the surface of Eno1 with a codon for cysteine, producing the cysteine added variant in a recombinant expression system, reacting the cysteine added variant with a cysteine-specific polymer reagent, and purifying the conjugated Eno1.

In this system, the addition of a polymer at the cysteine site can be accomplished through a maleimide active functionality on the polymer. Examples of this technology are provided infra. The amount of sulfhydryl reactive polymer used should be at least equimolar to the molar amount of cysteines to be derivatized and preferably is present in excess. In certain embodiments, at least a 5-fold molar excess of sulfhydryl reactive polymer is used, and in a further embodiment at least a ten-fold excess of such polymer is used. Other conditions useful for covalent attachment of the biocompatible polymer to Eno1 are within the skill of those in the art.

Accordingly, in certain aspects, the invention also relates to a method for the preparation of an Eno1 molecule, e.g., fusion protein, conjugated to a biocompatible polymer comprising mutating a nucleotide sequence that encodes for a functional Eno1 polypeptide to substitute a coding sequence for a cysteine residue; expressing the mutated nucleotide sequence to produce Eno1 with an added cysteine; purifying the Eno1; reacting the Eno1 with the biocompatible polymer that has been activated to react with polypeptides at substantially only reduced cysteine residues such that the conjugate is formed; and purifying the conjugate. In another embodiment, the invention provides a method for site-directed PEGylation of an Eno1 molecule, e.g., fusion protein, comprising: (a) expressing an Eno1 polypeptide (e.g., fusion protein) comprising an added cysteine residue, wherein the added cysteine is capped; (b) contacting the Eno1 cysteine variant with a reductant under conditions to mildly reduce the added cysteine residue and to release the cap; (c) removing the cap and the reductant from the Eno1 cysteine variant; and (d) at least about 5 minutes, at least 15 minutes, or at least 30 minutes after the removal of the reductant, treating the cysteine variant with PEG comprising a sulfhydryl coupling moiety under conditions such that PEGylated Eno1 is produced. The sulfhydryl coupling moiety of the PEG is selected from the group consisting of thiol, triflate, tresylate, aziridine, oxirane, S-pyridyl and maleimide moieties, preferably maleimide.

For example, in certain embodiments, PEG groups may be linked to a protein through a reactive functional group on an amino acid sidechain Amino acids suitable for such linkage include lysine, cysteine, histidine, arginine, aspartic acid, glutamic acid, serine, threonine, and tyrosine. PEG groups themselves have to be functionalized with a reactive group such as a maleimide, vinyl sulfone, pyridyl disulfide, amine, carboxylic acid, or NHS ester. One or more unpaired cysteine residues on Eno1 may be selectively reacted with a functionalized PEG group, such as a maleimide derivative, to form a pegylated conjugate, as depicted in Scheme 1 below:

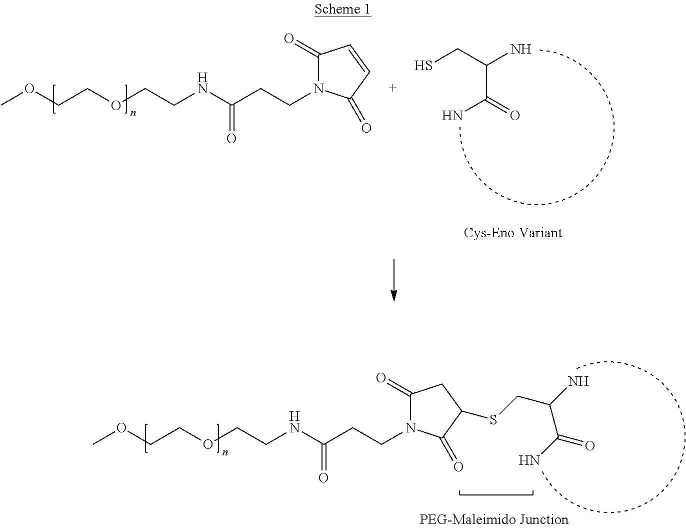

The process of pegylation is typically done using a solution phase batch process or on on-column fed batch process. The batch process generally is done by mixing reagents in a buffered solution held around 5° C. (±2° C.), followed by separation and purification by chromatography.

Accordingly, in some aspects, the invention also relates to an Eno1 protein, or fragment thereof, with one or more added cysteine residues as described above, wherein one or more of the cysteine residues is pegylated. In one embodiment of this aspect, the Eno1 protein is linked to one PEG group. In another embodiment of this aspect, the Eno1 protein is linked to two PEG groups. In a further embodiment of this aspect, the Eno1 protein is linked to three PEG groups. In a further embodiment of this aspect, the Eno1 protein is linked to 1, 2, 3, 4, 5 or 6 PEG groups.

In certain embodiments, the PEG group has a weight of between about 5 kDa to about 40 kDa. In certain embodiments, the PEG group has a weight of between about 5 kDa to about 20 kDa. In certain embodiments, the PEG group has a weight of about 5 kDa. In other embodiments, the PEG group has a weight of about 10 kDa. In further embodiments, the PEG group has a weight of about 20 kDa. In any of the above embodiments, the PEG group may be straight-chained or branched. In one embodiment, the PEG group is about 5 kDa or about 10 kDa and is branched. In another embodiment, the PEG group is about 10 kDa and linear. In another embodiment, the PEG group is about 20 kDa and is linear.

In certain embodiments, a PEG group is attached through a maleimide linker to one or more cysteine residues on the Eno 1 protein. In certain embodiments, a PEG group is attached through a maleimide linker to two cysteine residues on the Eno 1 protein. In one aspect of the previous embodiments, a PEG group(s) is attached to one or more cysteine residues at position 26, 78, 140, 236, 253, 267 and/or 418 of the Eno1 amino acid sequence of SEQ ID NO: 13. In another aspect of the previous embodiments, a PEG group(s) is attached to one or more cysteine residues at position 140, 267 and/or 418 of the Eno1 amino acid sequence of SEQ ID NO: 13.

In certain embodiments, the ratio of the Eno 1 to the PEG group is about 2:1 to about 1:5. In certain embodiments, the ratio of the Eno 1 to the PEG group is about 1.5:1, about 1:1, about 1:1.5, about 1:2, about 1:2.5 or about 1:3.

In certain aspects, the invention relates to a dimer comprising two Eno1 proteins or fragments thereof. In some embodiments, the dimer comprises or consists of one Eno1 protein comprising an added cysteine residue which is pegylated, and one Eno1 protein that is not pegylated and does not comprise an added cysteine reside (e.g. an endogenous Eno1 protein). In some embodiments, an Eno1 protein comprising a pegylated added cysteine residue is administered to a subject as a monomer, and this pegylated monomer forms a dimer with an endogenous Eno1 protein after administration to the subject.

### Targeted Eno1 Molecules

Delivery of drugs to their site of action can increase the therapeutic index by reducing the amount of drug required to provide the desired systemic effect. Drugs can be delivered to the site of action by administration of the drug to the target tissue using a method or formulation that will limit systemic exposure, e.g., intramuscular injection, intrasinovial injection, intrathecal injection, intraocular injection. A number of the sustained delivery formulations discussed above are for intramuscular administration and provide local delivery to muscle tissue. Alternatively, targeting moieties can be associated with or linked to therapeutic payloads for administration to the target site. Targeting moieties can include any of a number of moieties that bind to specific cell types.

1. Targeting Moieties

Certain embodiments of the invention include the use of targeting moieties include relatively small peptides (e.g., 25 amino acids or less, 20 amino acids or less, 15 amino acids or less, 10 amino acids or less), muscle targeting peptides (MTP) including smooth muscle and/or skeletal muscle targeting peptides, αvβ3 integrin ligands (e.g., RGD peptides and peptide analogs), αvrβ5 integrin ligands, or CD46 ligands as discussed above. It is understood that such peptides can include one or more chemical modifications to permit formation of a complex with Eno1, to modify pharmacokinetic and/or pharmacodynamic properties of the peptides. In certain embodiments, the targeting moiety can be a small molecule, e.g., RGD peptide mimetics. In certain embodiments, the targeting moiety can include a protein and optionally a fiber protein from an adenovirus 35. In certain embodiments, the viral proteins are present on a virus particle. In certain embodiments, the viral proteins are not present on a viral particle. In certain embodiments, the targeting moiety can be an antibody, antibody fragment, antibody mimetic, or T-cell receptor.

In certain embodiments, the targeting moiety is creatine. Creatine may be conjugated to Eno1 by making amide derivatives of creatine starting from a new protected creatine molecule ((Boc)₂-creatine). The creatine guanidine groups may be doubly Boc protected while allowing good reactivity of the carboxylic group of creatine. This temporary protection ensures efficient creatine dissolution in organic solvents and offers simultaneous protection of creatine from intramolecular cyclization to creatinine. In this manner, it is possible to selectively conjugate molecules (e.g. Eno1 protein or a fragment thereof) to creatine via the carboxylic group. The creatine guanidine group is easily deprotected at the end of the reaction, obtaining the desired creatine amide conjugate. In certain embodiments the targeting moiety is a muscle targeting peptide. Examples of muscle targeting peptides include, but are not limited to, ASSLNIA (SEQ ID NO: 7); WDANGKT (SEQ ID NO: 8); GETRAPL (SEQ ID NO: 9); CGHHPVYAC (SEQ ID NO: 5); and HAIYPRH (SEQ ID NO: 6).

2. Targeted Complexes

Targeted Eno1 complexes can be administered by a route other than intramuscular injection (e.g., subcutaneous injection, intravenous injection) while providing delivery of the Eno1 to muscle. Targeted complexes can include one or more targeting moieties attached either directly or indirectly to Eno1. Formation of the targeted complex does not substantially or irreversibly inhibit the activity of Eno1 and its effect on normalizing blood glucose levels and insulin response. In certain embodiments, use of a targeted complex can reduce the total amount of Eno1 required to provide an effective dose. Some exemplary, non-limiting, embodiments of targeted complexes are discussed below.

In certain embodiments, the Eno1 and the targeting moiety are present in an Eno1 molecule or complex at about a 1:1 molar ratio. In certain embodiments, the targeting moiety is present in an Eno1 molecule or complex with a molar excess of the payload (e.g., 2:1, 3:1, 4:1, 5:1, 6:1, 7:1; 8:1, 9:1, 10:1, 11:1, 12:1, 13:1, 14:1, 15:1, 16:1, 17:1; 18:1, 19:1, 20:1, 21:1, 22:1, 23:1, 24:1, 25:1, 26:1, 27:1; 28:1, 29:1, 30:1, or more; or any range bracketed by any two values). In certain embodiments, the payload to targeting moiety is about 1:5-1:15; about 1:7-1:13, about 1:8-1:12.

It is understood that the compositions and methods of the invention include the administration of more than one, i.e., a population of, targeting moiety-payload complexes. Therefore, it is understood that the number of targeting moieties per payload can represent an average number of targeting moieties per payload in a population of complexes. In certain embodiments, at least 70% of the complexes have the selected molar ratio of targeting moieties to payload. In certain embodiments, at least 75% of the complexes have the selected molar ratio of targeting moieties to payload. In certain embodiments, at least 80% of the complexes have the selected molar ratio of targeting moieties to payload. In certain embodiments, at least 85% of the complexes have the selected molar ratio of targeting moieties to payload. In certain embodiments, at least 90% of the complexes have the selected molar ratio of targeting moieties to payload.

3. Eno1 Muscle Targeted Fusion Proteins

In certain embodiments, the targeted Eno1 molecule is an Eno1 muscle targeted fusion protein. Eno1 muscle targeted fusion proteins may comprise a muscle targeting peptide, for example, ASSLNIA (SEQ ID NO: 7); WDANGKT (SEQ ID NO: 8); GETRAPL (SEQ ID NO: 9); CGHHPVYAC (SEQ ID NO: 5); and HAIYPRH (SEQ ID NO: 6). In certain embodiments, the muscle targeting peptide is attached to the N-terminus of Eno1. In other embodiments, the muscle targeting peptide is attached to the C-terminus of Eno1. The ENO1 muscle targeted fusion protein may also comprise a linker between the muscle targeting peptide and Eno1. In a particular embodiment, the linker comprises the amino acid sequence of SEQ ID NO: 14. In some embodiments, the Eno1 muscle targeted fusion protein comprises a peptide or protease tag comprising a protease cleavage site between the muscle targeting peptide and Eno1. In a particular embodiment, the peptide comprises the amino acid sequence of SEQ ID NO: 6. The Eno1 muscle targeted fusion protein may also comprise added cysteine residues as described above. In certain embodiments, the added cysteine residue is pegylated as described herein.

In certain embodiments, the Eno1 molecule comprises a ratio of muscle targeting peptide to Eno1 polypeptide of 1:1-5:1. In certain embodiments, the Eno1 molecule comprises a ratio of muscle targeting peptide to Eno1 polypeptide of 1:1. In certain embodiments, the Eno1 molecule comprises a ratio of muscle targeting peptide to Eno1 polypeptide of 2:1. In certain embodiments, the Eno1 molecule comprises a ratio of muscle targeting peptide to Eno1 polypeptide of 3:1. In certain embodiments, the Eno1 molecule comprises a ratio of muscle targeting peptide to Eno1 polypeptide of 4:1. In certain embodiments, the Eno1 molecule comprises a ratio of muscle targeting peptide to Eno1 polypeptide of 5:1.

4. Cell Penetrating Peptides

In some embodiments the complex comprising the Eno1 polypeptide further comprises a “cell penetrating peptide.” A “cell penetrating peptide” is capable of permeating a cell, e.g., a human cell. A microbial cell-permeating peptide can be, for example, an alpha-helical linear peptide (e.g., LL-37 or Ceropin P1), a disulfide bond-containing peptide (e.g., alpha-defensin, beta-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003). Suitable cell penetrating peptides include, but are not limited to, Penetratin (R6) (RQIKIWFQNRRMKWKK-NH2; (SEQ ID NO: 20) Derossi et al., 1994, J. Biol. Chem. 269:10444), HIV TAT, Transportan (AGYLLGK*INLKALAALAKKIL-NH2, SEQ ID NO: 21), Oligoarginine (R9) peptide, MPG peptide, KALA peptide, M918 (MVTVLFRRLRIRRACGPPRVRV-NH2, SEQ ID NO: 22), and YDEEGGGE-NH2 (SEQ ID NO: 23). Additional cell penetrating peptides are described, for example, in U.S. Pat. No. 8,796,436, the entire contents of which are incorporated herein by reference.

a. Linkers

A number of chemical linkers are known in the art and available from commercial sources (e.g., Pierce Thermo Fisher Scientific Inc., see, e.g., www.piercenet.com/cat/crosslinking-reagents). Such agents can be used to chemically link, reversibly or irreversibly, one or more functional moieties (e.g. an added cysteine, a targeting moiety, or a cell penetrating peptide) to Eno1. Linkers can also be used to attach targeting moieties and Eno1 to a structure, e.g., microparticle, dendrimer, rather than attaching the targeting moiety directly to Eno1. In some embodiments, a linker may be used to attach an added cysteine residue to Eno1, for example, to the N-terminus or C-terminus of Eno1. In certain embodiments, the linker attaching Eno1 to the functional moiety is reversible so that the Eno1 is released from the complex after administration, preferably substantially at the muscle.

In some embodiments, the linker is a serine linker, i.e. a linker comprising 1, 2, 3, 4, 5, 6, 7, 8, 9, 10, 11, 12, 13, 14, 15, 16, 17, 18, 19, 20 or more contiguous serine residues. In some embodiments, the linker is a glycine linker, i.e. a linker comprising 1, 2, 3, 4, 5, 6, 7, 8, 9, 10, 11, 12, 13, 14, 15, 16, 17, 18, 19, 20 or more contiguous glycine residues. In some embodiments, the linker is a glycine-serine linker i.e. a linker comprising 1, 2, 3, 4, 5, 6, 7, 8, 9, 10, 11, 12, 13, 14, 15, 16, 17, 18, 19, 20 or more contiguous serine and glycine residues. In a particular embodiment, the glycine-serine linker comprises 1, 2, 3, 4, 5, 6, 7, 8, 9, 10, 11, 12, 13, 14, 15, 16, 17, 18, 19, 20 or more contiguous repeats of the sequence GGS (glycine-glycine-serine). In a further particular embodiment, the glycine-serine linker comprises SEQ ID NO: 14.

b. Peptide Bonds

As used herein, targeted complexes can include the translation of Eno1 with a peptide targeting moiety and/or cell penetrating peptide. Methods to generate expression constructs including an amino acid sequence for targeting Eno1 is well within the ability of those of skill in the art.

c. Liposomes

Liposomal delivery systems are known in the art including formulations to limit systemic exposure, thereby reducing systemic exposure and off target effects. For example, Doxil® is a composition in which doxorubicin encapsulated in long-circulating pegylated liposomes that further comprise cholesterol for treatment of certain types of cancer. Various liposomal formulations of amphotericin B including Ambisome®, Abelcet®, and Amphotec® are formulated for intravenous administration in liposomes or a lipid complex containing various phospholipids, cholesterol, and cholesteryl sulfate. Visudine® is verteporfin formulated as a liposome in egg phosphotidyl glycerol and DMPC for intravenous administration. Liposomal formulations are also known for intramuscular injection. Epaxal® is an inactivated hepatitis A virus and Inflexal V® is an inactivated hemaglutinine of influenza virus strains A and B. Both viral preparations are formulated in combinations of DOPC and DOPE. Such liposomes, or other physiologically acceptable liposomes, can be used for the packaging of Eno1 and subsequent surface decoration with targeting moieties to delivery Eno1 to the muscle. Additional moieties to modulate intracellular trafficking of the liposome can also be included. Upon uptake of the liposome into the cell, the liposome releases the Eno1 thereby allowing it to have its therapeutic effect.

### Eno1 Activity

Eno1 is a key glycolytic enzyme that catalyzes the dehydratation of 2-phospho-D-glycerate (PGA) to phosphoenolpyruvate (PEP) in the last steps of the catabolic glycolytic pathway. Diaz-Ramos et al., 2012, J Biomed Biotechnol. 2012: 156795. Enolase enzymes catalyse the dehydration of PGA to PEP in the Emden Mayerhoff-Parnas glycolytic pathway (catabolic direction). In the anabolic pathway (reverse reaction) during gluconeogenesis, Eno1 catalyses hydration of PEP to PGA. Accordingly Eno1 is also known as phosphopyruvate hydratase. Metal ions are cofactors impairing the increase of enolase activity; hence Eno1 is also called metal-activated metalloenzyme. Magnesium is a natural cofactor causing the highest activity and is required for the enzyme to be catalytically active. The relative activation strength profile of additional metal ions involved in the enzyme activity appears in the following order Mg²⁺>Zn²⁺>Mn²⁺>Fe(II)²⁺>Cd²⁺>Co²⁺, Ni²⁺, Sm³⁺, Tb³⁺ and most other divalent metal ions. In reactions catalyzed by enolases, the alpha-proton from a carbon adjacent to a carboxylate group of PGA, is abstracted, and PGA is conversed to enolate anion intermediate. This intermediate is further processed in a variety of chemical reactions, including racemization, cycloisomerization and beta-elimination of either water or ammonia. See Atlas of Genetics and Cytogenetics in Oncology and Haematology database, atlasgeneticsoncology.org/Genes/GC_ENO1.html.

Enzymatically active enolase exists in a dimeric (homo- or heterodimers) form and is composed of two subunits facing each other in an antiparallel fashion. The crystal structure of enolase from yeast and human has been determined and catalytic mechanisms have been proposed. (Diaz-Ramos et al., cited above.) The five residues that participate in catalytic activity of this enzyme are highly conserved throughout evolution. Studies in vitro revealed that mutant enolase enzymes that differ at positions Glu168, Glu211, Lys345, Lys396 or His159, demonstrate dramatically decreased activity levels. An integral and conserved part of enolases are two Mg2+ ions that participate in conformational changes of the active site of enolase and enable binding of a substrate or its analogues. (Atlas of Genetics and Cytogenetics in Oncology database, cited above.) Accordingly, in certain embodiments, the compositions of the invention further comprise a metal ion cofactor. The metal ion cofactor can provide increased stability of the Eno1 in the composition and/or increased activity of the Eno1 in vivo. In one embodiment, the metal ion cofactor is divalent. In one embodiment, the divalent metal ion cofactor is Mg²⁺, Zn²⁺, Mn²⁺, Fe(II)²⁺, Cd²⁺, Co²⁺, or Ni²⁺. In one embodiment, the metal ion cofactor is trivalent, e.g. Sm³⁺ or Tb3⁺.

Eno1 activity may be determined, for example, using the pyruvate kinase (PK)/lactate dehydrogenase (LDH) assay. The reaction for this enolase assay is shown below.

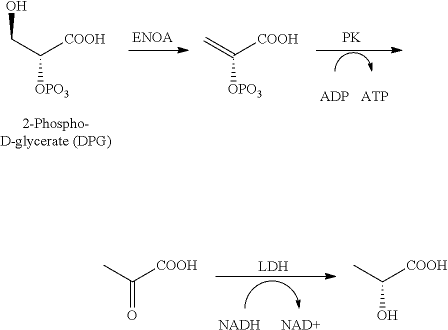

The rate of reaction of NADH to NAD⁺ conversion may be determined by measuring the decrease of fluorescence of NADH, for example by using a PTI Quantamaster 40 spectrophotometer from Photon Technology International, Inc. (pti-nj.com). Kits for measuring Eno1 activity by a colorimetric pyruvate kinase/lactate dehydrogenase assay are also commercially available, for example, from ABCAM (Cambridge, Mass.; Cat. No. ab117994). The ABCAM Eno1 activity assay is further described in Example 5 below.

Eno1 activity may also be determined by measuring the effect of Eno1 on glucose uptake in human skeletal muscle myotubes (HSMM) as described in Example 2.

In certain embodiments, the Eno1 or the fragment thereof has at least 10%, 20%, 30%, 40%, 50%, 60%, 70%, 80%, 90%, 100%, 110%, 120%, 130%, 140%, 150%, 160%, 170%, 180%, 190%, 200%, 300%, 400% or 500% of the activity of a purified endogenous human Eno1 polypeptide. In certain embodiments, the activity of the Eno1, the fragment thereof, and the purified endogenous human Eno1 polypeptide are determined by the pyruvate kinase/lactate dehydrogenase assay or the HSMM glucose uptake assay described above.

In certain embodiments, the Eno1 polypeptide in complex with a muscle targeting moiety (e.g. a muscle targeting peptide) as described herein has at least 10%, 20%, 30%, 40%, 50%, 60%, 70%, 80%, 90%, 100%, 110%, 120%, 130%, 140%, 150%, 160%, 170%, 180%, 190%, 200%, 300%, 400% or 500% of the activity of a purified endogenous Eno1 polypeptide that is not in complex with a muscle targeting moiety. In certain embodiments, the activity of the Eno1 polypeptide in complex with a muscle targeting moiety and the activity of the purified endogenous Eno1 polypeptide that is not in complex with a muscle targeting moiety are determined by the pyruvate kinase/lactate dehydrogenase assay or the HSMM glucose uptake assay described above.

In certain embodiments, the Eno1 muscle targeted fusion protein as described herein has at least 10%, 20%, 30%, 40%, 50%, 60%, 70%, 80%, 90%, 100%, 110%, 120%, 130%, 140%, 150%, 160%, 170%, 180%, 190%, 200%, 300%, 400% or 500% of the activity of a purified endogenous Eno1 polypeptide that is not fused with a muscle targeting peptide. In certain embodiments, the activity of the Eno1 muscle targeted fusion protein and the activity of the purified endogenous Eno1 polypeptide that is not fused with a muscle targeting peptide are determined by the pyruvate kinase/lactate dehydrogenase assay or the HSMM glucose uptake assay described above.

In certain embodiments the pegylated Eno1 polypeptide as described herein has at least 10%, 20%, 30%, 40%, 50%, 60%, 70%, 80%, 90%, 100%, 110%, 120%, 130%, 140%, 150%, 160%, 170%, 180%, 190%, 200%, 300%, 400% or 500% of the activity of a purified endogenous ENO1 polypeptide that is not pegylated. In certain embodiments the activity of the pegylated Eno1 polypeptide and the activity of the purified endogenous ENO1 polypeptide that is not pegylated are determined by the pyruvate kinase/lactate dehydrogenase assay or the HSMM glucose uptake assay described above.

In certain embodiments, the Eno1 muscle targeted fusion protein which is PEGylated as described herein has at least 10%, 20%, 30%, 40%, 50%, 60%, 70%, 80%, 90%, 100%, 110%, 120%, 130%, 140%, 150%, 160%, 170%, 180%, 190%, 200%, 300%, 400% or 500% of the activity of a purified endogenous Eno1 polypeptide that is not fused with a muscle targeting peptide or PEGylated. In certain embodiments, the activity of the Eno1 muscle targeted fusion protein which is PEGylated and the activity of the purified endogenous Eno1 polypeptide that is not fused with a muscle targeting peptide or PEGylated are determined by the pyruvate kinase/lactate dehydrogenase assay or the HSMM glucose uptake assay described above.

In one embodiment, the Eno1 or the fragment thereof in the composition of the invention, wherein the composition comprises a metal ion cofactor (e.g., a divalent metal ion cofactor, e.g., Mg²⁺, Zn²⁺, Mn²⁺, Fe(II)²⁺, Cd²⁺, Co²⁺, or Ni²⁺, or a trivalent metal ion cofactor, e.g. Sm³⁺ or Tb3⁺⁾ has at least 10%, 20%, 30%, 40%, 50%, 60%, 70%, 80%, 90%, 100%, 110%, 120%, 130%, 140%, 150%, 160%, 170%, 180%, 190%, 200%, 300%, 400% or 500% of the activity of a purified endogenous human Eno1 polypeptide. In certain embodiments, the activity of the Eno1 or the fragment thereof in the composition comprising a metal ion cofactor as described above and the activity of the purified endogenous human Eno1 polypeptide are determined by the pyruvate kinase/lactate dehydrogenase assay or the HSMM glucose uptake assay described above.

Glucose Flux

The regulation of muscle glucose uptake involves a three-step process consisting of: (1) delivery of glucose to muscle, (2) transport of glucose into the muscle by the glucose transporter GLUT4 and (3) phosphorylation of glucose within the muscle by a hexokinase (HK). The physiological regulation of muscle glucose uptake requires that glucose travels from the blood to the interstitium to the intracellular space and is then phosphorylated to G6P. Blood glucose concentration, muscle blood flow and recruitment of capillaries to muscle determine glucose movement from the blood to the interstitium. Plasma membrane GLUT4 content controls glucose transport into the cell. Muscle hexokinase (HK) activity, cellular HK compartmentalization and the concentration of the HK inhibitor, G6P, determine the capacity to phosphorylate glucose. These three steps—delivery, transport and phosphorylation of glucose—comprise glucose flux, and all three steps are important for glucose flux control. However steps downstream of glucose phosphorylation may also affect glucose uptake. For example, acceleration of glycolysis or glycogen synthesis could reduce G6P, increase HK activity, increase the capacity for glucose phosphorylation and potentially stimulate muscle glucose uptake. Wasserman et al., 2010, J Experimental Biology, Vol. 214, pp. 254-262.

The present invention provides methods for treatment of elevated blood glucose typically related to diabetes including at least type 1 diabetes, pre-diabetes, type 2 diabetes, and gestational diabetes by administration of Eno1 to the subject. The invention also provides methods for increasing glucose flux in a subject comprising administering to the subject a pharmaceutical composition comprising Eno1 or a fragment thereof. In certain embodiments, the pharmaceutical composition administered to the subject is any of the pharmaceutical compositions described herein. The invention also provides a method of increasing glucose flux in a skeletal muscle cell of a subject, the method comprising administering to the subject a pharmaceutical composition comprising Eno1 or a fragment thereof. In certain embodiments, the pharmaceutical composition administered to the subject is any of the pharmaceutical compositions described herein.

The invention also provides a method of increasing glycolytic activity in a skeletal muscle cell of a subject, the method comprising administering to the subject a pharmaceutical composition comprising Eno1 or a fragment thereof. In certain embodiments, the pharmaceutical composition administered to the subject is any of the pharmaceutical compositions described herein.

The invention also provides a method of increasing mitochondrial free fatty acid oxidation in a skeletal muscle cell of a subject, the method comprising administering to the subject a pharmaceutical composition comprising Eno1 or a fragment thereof. In certain embodiments, the pharmaceutical composition administered to the subject is any of the pharmaceutical compositions described herein.

“Increasing glucose flux” as used herein is understood as increasing at least one or more of (1) delivery of glucose to muscle, (2) transport of glucose into the muscle, and (3) phosphorylation of glucose within the muscle. In particular embodiments, increasing glucose flux includes increasing glycolytic activity or mitochondrial free fatty acid oxidation in a muscle cell.

### III. DIABETES DIAGNOSIS AND CLASSIFICATION

Diabetes mellitus (DM), often simply referred to as diabetes, is a group of metabolic diseases in which a person has high blood sugar, either because the body does not produce enough insulin or because cells do not respond to the insulin that is produced. This high blood sugar produces the classical symptoms of polyuria (frequent urination), polydipsia (increased thirst), and polyphagia (increased hunger).

Type 2 diabetes results from insulin resistance, a condition in which cells fail to use insulin properly, sometimes combined with an absolute insulin deficiency. The defective responsiveness of body tissues to insulin is believed, at least in part, to involve the insulin receptor. However, the specific defects are not known.

In the early stage of type 2 diabetes, the predominant abnormality is reduced insulin sensitivity. At this stage, hyperglycemia can be reversed by a variety of measures and medications that improve insulin sensitivity or reduce glucose production by the liver. Prediabetes indicates a condition that occurs when a person's blood glucose levels are higher than normal but not high enough for a diagnosis of type 2 diabetes.

Type 2 diabetes is due to insufficient insulin production from beta cells in the setting of insulin resistance. Insulin resistance, which is the inability of cells to respond adequately to normal levels of insulin, occurs primarily within the muscles, liver, and fat tissue. In the liver, insulin normally suppresses glucose release. However in the setting of insulin resistance, the liver inappropriately releases glucose into the blood. The proportion of insulin resistance verses beta cell dysfunction differs among individuals with some having primarily insulin resistance and only a minor defect in insulin secretion and others with slight insulin resistance and primarily a lack of insulin secretion.

Other potentially important mechanisms associated with type 2 diabetes and insulin resistance include: increased breakdown of lipids within fat cells, resistance to and lack of incretin, high glucagon levels in the blood, increased retention of salt and water by the kidneys, and inappropriate regulation of metabolism by the central nervous system. However not all people with insulin resistance develop diabetes, since an impairment of insulin secretion by pancreatic beta cells is also required.

Type 1 diabetes results from the body's failure to produce insulin, and presently requires treatment with injectable insulin. Type 1 diabetes is characterized by loss of the insulin-producing beta cells of the islets of Langerhans in the pancreas, leading to insulin deficiency. Most affected people are otherwise healthy and of a healthy weight when onset occurs. Sensitivity and responsiveness to insulin are usually normal, especially in the early stages. However, particularly in late stages, insulin resistance can occur, including insulin resistance due to immune system clearance of administered insulin.

### Diagnostic Criteria

Criteria for diagnosis and classification of diabetes mellitus were published by the American Diabetes Association in Diabetes Care, 36:S67-74, 2013, incorporated herein by reference, which provides a more detailed definition of the various types of diabetes. Diagnostic criteria for diabetes are discussed further below. The reference classifies type 1 diabetes or type 2 diabetes as follows:
I. Type 1 diabetes (β-cell destruction, usually leading to absolute insulin deficiency)
   A. Immune mediated
   B. Idiopathic
II. Type 2 diabetes (may range from predominantly insulin resistance with relative insulin deficiency to a predominantly secretory defect with insulin resistance)
III. Other specific types
IV. Gestational diabetes mellitus

Methods for performing diagnostic or assessment methods are provided therein. The diagnostic criteria for diabetes provided therein are as follows:

Criteria for the Diagnosis of Diabetes

HbA1c ≧ 6.5%. The test should be performed in a laboratory using a
method that is National Glycohemoglobin Standardization Program
(NGSP) certified and standardized to the Diabetes Control and
Complications Trial (DCCT) assay.*
OR
Fasting plasma glucose (FPG) ≧126 mg/dl (7.0 mmol/l). Fasting is
defined as no caloric intake for at least 8 h.*
OR
2-h plasma glucose ≧200 mg/dl (11.1 mmol/l) during an oral glucose
tolerance test (OGTT). The test should be performed as described by
the World Health Organization, using a glucose load containing the
equivalent of 75 g anhydrous glucose dissolved in water.*
OR
In a patient with classic symptoms of hyperglycemia or hyperglycemic
crisis, a random plasma glucose ≧200 mg/dl (11.1 mmol/l).
*In the absence of unequivocal hyperglycemia, criteria 1-3 should be confirmed by repeat testing.

The diagnostic criteria for increased risk of diabetes/pre-diabetes provided therein are as follows:

Criteria for Increased Risk of Diabetes (Pre-Diabetes)*

Fasting Plasma Glucose (FPG) 100 mg/dl (5.6 mmol/l) to 125 mg/dl
(6.9 mmol/l) [Impaired Fasting Glucose—IFG]
2-h Plasma Glucose (PG) in the 75-g oral glucose tolerance test
(OGTT) 140 mg/dl (7.8 mmol/l) to 199 mg/dl (11.0 mmol/l)
[Impaired Glucose Tolerance—IGT]
A1C 5.7-6.4%
*For all three tests, risk is continuous, extending below the lower limit of the range and becoming disproportionately greater at higher ends of the range.

The diagnostic criteria for gestational diabetes provided therein are as follows:

Screening for and diagnosis of Gestational
Diabetes Mellitus (GDM)

Perform a 75-g OGTT, with plasma glucose measurement fasting and at 1
and 2 h, at 24-28 weeks of gestation in women not previously diagnosed
with overt diabetes.
The OGTT should be performed in the morning after an overnight fast of
at least 8 h.
The diagnosis of GDM is made when any of the following plasma glucose
values are exceeded:

Fasting: ≧92 mg/dl (5.1 mmol/1)
1 h: ≧180 mg/dl (10.0 mmol/l)
2 h: ≧153 mg/dl (8.5 mmol/l)

The blood glucose measurements for the diagnosis and/or monitoring of elevated blood glucose or diabetes can be cumbersome due to the specific timing requirements relative to eating, e.g., a fasting blood glucose or the amount of time required to perform the test, e.g., as with an oral glucose tolerance test. Moreover, the diagnostic criteria explicitly require that in absence of unequivocal hyperglycemia, criteria 1-3 should be confirmed by repeat testing. The use of an HbA1c level as a diagnostic indicator can be advantageous as it provides an indication of blood glucose levels over time, i.e., for about the prior 1-2 months, and does not require special scheduling to perform the test. Similarly, an Eno1 level can be determined without particular scheduling requirements or food consumption limitations or requirements.

### Secondary Pathologies of Diabetes, Insulin Resistance, and Insulin Insufficiency

Abnormal glucose regulation resulting from diabetes, both type 1 and type 2, insulin resistance, and insulin insufficiency are associated with secondary pathologies, many of which result from poor circulation. Such secondary pathologies include macular degeneration, peripheral neuropathies, ulcers and decrease wound healing, and decreased kidney function. It has been suggested that maintaining glucose levels and/or HbAc1 levels within normal ranges decreases the occurrence of these secondary pathologies. It is understood that normalization of blood glucose, insulin, and HbAc1 levels will reduce the development of secondary pathologies by limiting the primary pathology, e.g., impaired glucose tolerance, increased blood glucose. In certain embodiments, Eno1 is not used for the treatment of secondary pathologies associated with impaired glucose tolerance, increased blood glucose, insulin resistance, insulin insufficiency, diabetes, or pre-diabetes. In certain embodiments, Eno1 is used for the treatment of secondary pathologies associated with impaired glucose tolerance, increased blood glucose, insulin resistance, insulin insufficiency, diabetes, or pre-diabetes.

### IV. OBESITY AND DIABETES

Obesity (commonly defined as a Body Mass Index of approximately >30 kg/m2) is often associated with a variety of pathologic conditions such as hyperinsulinemia, insulin resistance, diabetes, hypertension, and dyslipidemia. Each of these conditions contributes to the risk of cardiovascular disease.

Along with insulin resistance, hypertension, and dyslipidemia, obesity is considered to be a component of the Metabolic Syndrome (also known as Syndrome X) which together synergize to potentiate cardiovascular disease. More recently, the U.S. National Cholesterol Education Program has classified Metabolic Syndrome as meeting three out of the following five criteria: fasting glucose level of at least 110 mg/dl, plasma triglyceride level of at least 150 mg/dl (hypertriglycerdemia), HDL cholesterol below 40 mg/dl in men or below 50 mg/dl in women, blood pressure at least 130/85 mm Hg (hypertension), and central obesity, with central obesity being defined as abdominal waist circumference greater than 40 inches for men and greater than 35 inches for women.

Diabetes mellitus (DM), often simply referred to as diabetes, is a group of metabolic diseases in which a person has high blood sugar, either because the body does not produce enough insulin or because cells do not respond to the insulin that is produced. This high blood sugar produces the classical symptoms of polyuria (frequent urination), polydipsia (increased thirst), and polyphagia (increased hunger).

Type 2 diabetes results from insulin resistance, a condition in which cells fail to use insulin properly, sometimes combined with an absolute insulin deficiency. The defective responsiveness of body tissues to insulin is believed, at least in part, to involve the insulin receptor. However, the specific defects are not known.

In the early stage of type 2 diabetes, the predominant abnormality is reduced insulin sensitivity. At this stage, hyperglycemia can be reversed by a variety of measures and medications that improve insulin sensitivity or reduce glucose production by the liver. Prediabetes indicates a condition that occurs when a person's blood glucose levels are higher than normal but not high enough for a diagnosis of type 2 diabetes.

Type 2 diabetes is due to insufficient insulin production from beta cells in the setting of insulin resistance. Insulin resistance, which is the inability of cells to respond adequately to normal levels of insulin, occurs primarily within the muscles, liver, and fat tissue. In the liver, insulin normally suppresses glucose release. However in the setting of insulin resistance, the liver inappropriately releases glucose into the blood. The proportion of insulin resistance verses beta cell dysfunction differs among individuals with some having primarily insulin resistance and only a minor defect in insulin secretion and others with slight insulin resistance and primarily a lack of insulin secretion.

Other potentially important mechanisms associated with type 2 diabetes and insulin resistance include: increased breakdown of lipids within fat cells, resistance to and lack of incretin, high glucagon levels in the blood, increased retention of salt and water by the kidneys, and inappropriate regulation of metabolism by the central nervous system. However not all people with insulin resistance develop diabetes, since an impairment of insulin secretion by pancreatic beta cells is also required.

Type 1 diabetes results from the body's failure to produce insulin, and presently requires treatment with injectable insulin. Type 1 diabetes is characterized by loss of the insulin-producing beta cells of the islets of Langerhans in the pancreas, leading to insulin deficiency. Most affected people are otherwise healthy and of a healthy weight when onset occurs. Sensitivity and responsiveness to insulin are usually normal, especially in the early stages. However, particularly in late stages, insulin resistance can occur, including insulin resistance due to immune system clearance of administered insulin.

### V. DOSAGES AND MODES OF ADMINISTRATION

Techniques and dosages for administration vary depending on the type of compound (e.g., protein and/or nucleic acid, alone or complexed with a microparticle, liposome, or dendrimer) and are well known to those skilled in the art or are readily determined

Therapeutic compounds of the present invention may be administered with a pharmaceutically acceptable diluent, carrier, or excipient, in unit dosage form. Administration may be parenteral, intravenous, subcutaneous, oral, topical, or local. In certain embodiments, administration is not oral. In certain embodiments, administration is not topical. In certain preferred embodiments, administration is systemic. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, subcutaneous delivery, etc.

The composition can be in the form of a pill, tablet, capsule, liquid, or sustained release tablet for oral administration; or a liquid for intravenous, subcutaneous, or parenteral administration; or a polymer or other sustained release vehicle for systemic administration.

Methods well known in the art for making formulations are found, for example, in “Remington: The Science and Practice of Pharmacy” (20th ed., ed. A. R. Gennaro, 2000, Lippincott Williams & Wilkins, Philadelphia, Pa.). Formulations for parenteral administration may, for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Nanoparticulate formulations (e.g., biodegradable nanoparticles, solid lipid nanoparticles, liposomes) may be used to control the biodistribution of the compounds. Other potentially useful parenteral delivery systems include ethylene-vinyl acetate copolymer particles, osmotic pumps, implantable infusion systems, and liposomes. The concentration of the compound in the formulation varies depending upon a number of factors, including the dosage of the drug to be administered, and the route of administration.

The compound may be optionally administered as a pharmaceutically acceptable salt, such as non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids and the like; polymeric acids such as tannic acid, carboxymethyl cellulose, and the like; and inorganic acid such as hydrochloric acid, hydrobromic acid, sulfuric acid phosphoric acid, and the like. Metal complexes include zinc, iron, and the like.

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and anti-adhesives (e.g., magnesium stearate, zinc stearate, stearic acid, silicas, hydrogenated vegetable oils, or talc). Formulations for oral use may also be provided as chewable tablets, or as hard gelatin capsules wherein the active ingredient is mixed with an inert solid diluent, or as soft gelatin capsules wherein the active ingredient is mixed with water or an oil medium.

The dosage and the timing of administering the compound depend on various clinical factors including the overall health of the subject and the severity of the symptoms of disease, e.g., diabetes, pre-diabetes.

### Formulations for Long Acting Injectable Drugs

Biologics and other agents subject to high rates of first pass clearance may not be amenable to oral administration and require administration by parenteral routes. However, compliance with treatment regimens for injectable drugs can be low as subjects are often adverse to self-administering agents by injection, e.g., subcutaneous injection, particularly when the disease does not make the subject feel sick. Other routes of administration by injection, e.g., intravenous, intramuscular, typically require administration by a trained professional, making frequent administration of the agent inconvenient and often painful.

Formulations have been created to provide sustained delivery of injectable agents including, but not limited to, oil-based injections, injectable drug suspensions, injectable microspheres, and injectable in situ systems. Long-acting injectable formulations offer many advantages when compared with conventional formulations of the same compounds. These advantages include, at least, the following: a predictable drug-release profile during a defined period of time following each injection; better patient compliance; ease of application; improved systemic availability by avoidance of first-pass metabolism; reduced dosing frequency (i.e., fewer injections) without compromising the effectiveness of the treatment; decreased incidence of side effects; and overall cost reduction of medical care.

1. Oil-Based Injectable Solutions and Injectable Drug Suspensions.

Conventional long-acting injections consist either of lipophilic drugs in aqueous solvents as suspensions or of lipophilic drugs dissolved in vegetable oils. Commercially available oil based injectable drugs for intramuscular administration include, but are not limited to, haloperidol deconate, fluphenazine deconate, testosterone enanthate, and estradiol valerate. Administration frequency for these long-acting formulations is every few weeks or so. In the suspension formulations, the rate-limiting step of drug absorption is the dissolution of drug particles in the formulation or in the tissue fluid surrounding the drug formulation. Poorly water-soluble salt formations can be used to control the dissolution rate of drug particles to prolong the absorption. However, several other factors such as injection site, injection volume, the extent of spreading of the depot at the injection site, and the absorption and distribution of the oil vehicle per se can affect the overall pharmacokinetic profile of the drug. Modulation of these factors to provide the desired drug release profile is within the ability of those of skill in the art.

2. Polymer-Based Microspheres and In-Situ Formings.

The development of polymer-based long-acting injectables is one of the most suitable strategies for macromolecules such as peptide and protein drugs. Commercially available microsphere preparations include, but are not limited to, leuprolide acetate, triptorelin pamoate, octreotide acetate, lanreotide acetate, risperidone, and naltrexone. Commercially available in situ forming implants include leuprolide acetate, and in situ forming implants containing paclitaxel and bupivacaine are in clinical trials. These formulations are for intramuscular administration. Advantages of polymer-based formulations for macromolecules include: in vitro and in vivo stabilization of macromolecules, improvement of systemic availability, extension of biological half life, enhancement of patient convenience and compliance, and reduction of dosing frequency.

The most crucial factor in the design of injectable microspheres and in situ formings is the choice of an appropriate biodegradable polymer. The release of the drug molecule from biodegradable microspheres is controlled by diffusion through the polymer matrix and polymer degradation. The nature of the polymer, such as composition of copolymer ratios, polymer crystallinities, glass-transition temperature, and hydrophilicities plays a critical role in the release process. Although the structure, intrinsic polymer properties, core solubility, polymer hydrophilicity, and polymer molecular weight influence the drug-release kinetics, the possible mechanisms of drug release from microsphere are as follows: initial release from the surface, release through the pores, diffusion through the intact polymer barrier, diffusion through a water-swollen barrier, polymer erosion, and bulk degradation. All these mechanisms together play a part in the release process. Polymers for use in microsphere and in situ formings include, but are not limited to a variety of biodegradable polymers for controlled drug delivery intensively studied over the past several decades include polylactides (PLA), polyglycolides (PGA), poly(lactide-co-glycolide) (PLGA), poly(ε-caprolactone) (PCL), polyglyconate, polyanhydrides, polyorthoesters, poly(dioxanone), and polyalkylcyanoacrylates. Thermally induced gelling systems used in in situ formings show thermo-reversible sol/gel transitions and are characterized by a lower critical solution temperature. They are liquid at room temperature and produce a gel at and above the lower critical solution temperature. In situ solidifying organogels are composed of water-insoluble amphiphilic lipids, which swell in water and form various types of lyotropic liquid crystals.

### VI. Detection and Measurement of Indicators of Blood Glucose Levels and Control

Methods for detection and measurement of indicators of elevated blood glucose and blood glucose control vary depending on the nature of the indicator to be measured. Elevated blood glucose, and thereby loss of blood glucose level control and severity of diabetes can be measured directly, e.g., by determining the amount of glucose in the blood, or indirectly, e.g., by detecting the amount of glycated hemoglobin (HbA1c), a reaction product of hemoglobin and glucose.

The present invention contemplates any suitable means, techniques, and/or procedures for detecting and/or measuring the blood glucose level indicators of the invention. The skilled artisan will appreciate that the methodologies employed to measure the indicators of the invention will depend at least on the type of indicator being detected or measured (e.g., glucose, ketones, mRNA, or polypeptide including a glycated polypeptide) and the biological sample (e.g., whole blood, serum). Certain biological sample may also require certain specialized treatments prior to measuring the biomarkers of the invention, e.g., the preparation of mRNA in the case where an mRNA biomarker, e.g., Eno1 mRNA, is being measured.

### Direct and Indirect Measurement of Blood Glucose and Blood Glucose Control Using Established Indicators

Blood glucose monitoring is a way of testing the concentration of glucose in the blood (glycemia) directly at a single point in time. Particularly important in the care of diabetes mellitus, a blood glucose test is performed by piercing the skin (typically, on the finger) to draw blood, then applying the blood to a chemically active disposable ‘test-strip’. Different manufacturers use different technology, but most systems measure an electrical characteristic, and use this to determine the glucose level in the blood. The test is usually referred to as capillary blood glucose. Commercially available blood glucose monitors for periodic or continuous use are known in the art. Glucose monitors for periodic detection of blood glucose levels include, but are not limited to, TRUEResult Blood Glucose Meter (TRUE), ACCU-CHEK Glucose Meter (ACCU-CHEK), OneTouch Glucose Meter (ONETOUCH), and FreeStyle Lite Blood Glucose (FREESTYLE LITE). It is understood that a directly measured normal blood glucose level will vary depending on the amount of time since food was last consumed with a normal fasting blood glucose level being lower than a normal fed blood glucose level. Direct blood glucose monitoring is also used in glucose tolerance tests to monitor response to consumption of a high dose of glucose and the rate of glucose clearance from the blood.

Glycated hemoglobin (hemoglobin A1c, HbA1c, A1C, Hb1c, HbA1c) is a form of hemoglobin that is measured primarily to identify the average plasma glucose concentration over prolonged periods of time, i.e., an indirect measurement of blood glucose. HbA1c is formed in a non-enzymatic glycation pathway by hemoglobin's exposure to plasma glucose. When normal levels of glucose are present, a normal amount of glycated hemoglobin, measured as a percent of total hemoglobin, or a specific blood concentration, is produced. When blood glucose levels are high, elevated levels of glycated hemoglobin are produced. Glycation is an irreversible reaction. Therefore, the amount of glycated hemoglobin within the red cell reflects the average level of glucose to which the cell has been exposed. Measuring glycated hemoglobin assesses the effectiveness of therapy by monitoring long-term serum glucose regulation rather than a snapshot image as provided by glucose monitoring. The HbA1c level is proportional to average blood glucose concentration over the previous four weeks to three months. HbA1c levels can be measured, for example, using high-performance liquid chromatography (HPLC) or immunoassay. Methods for detection and measurement of protein analytes are discussed in detail below.

1. Isolated Nucleic Acid Indicators

One aspect of the invention pertains to isolated nucleic acid molecules, including nucleic acids which encode Eno1 or a portion thereof. Isolated nucleic acids of the invention also include nucleic acid molecules sufficient for use as hybridization probes to identify Eno1 nucleic acid molecules, and fragments thereof, e.g., those suitable for use as PCR primers for the amplification of a specific product or mutation of marker nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. In one embodiment, an “isolated” nucleic acid molecule (preferably a protein-encoding sequences) is free of sequences which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. In another embodiment, an “isolated” nucleic acid molecule, such as a cDNA molecule, can be substantially free of other cellular material, or culture medium when produced by recombinant techniques, or substantially free of chemical precursors or other chemicals when chemically synthesized. A nucleic acid molecule that is substantially free of cellular material includes preparations having less than about 30%, 20%, 10%, or 5% of heterologous nucleic acid (also referred to herein as a “contaminating nucleic acid”).

A nucleic acid molecule of the present invention can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A nucleic acid molecule of the invention can be amplified using cDNA, mRNA, or genomic DNA as a template and appropriate oligonucleotide primers according to standard PCR amplification techniques. The nucleic acid so amplified can be cloned into an appropriate vector and characterized by DNA sequence analysis. Furthermore, nucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises an Eno1 molecule which has a nucleotide sequence complementary to the nucleotide sequence of a marker nucleic acid or to the nucleotide sequence of a nucleic acid encoding Eno1. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence, wherein the full length nucleic acid sequence comprises an Eno1 nucleic acid or which encodes an Eno1 protein. The invention further encompasses nucleic acid molecules that differ, due to degeneracy of the genetic code, from the nucleotide sequence of nucleic acids encoding Eno1 protein (e.g., protein having the sequence provided in the sequence listing), and thus encode the same protein.

It will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

As used herein, the phrase “allelic variant” refers to a nucleotide sequence which occurs at a given locus or to a polypeptide encoded by the nucleotide sequence.

As used herein, the terms “gene” and “recombinant gene” refer to nucleic acid molecules comprising an open reading frame encoding a polypeptide corresponding to an indicator of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

In another embodiment, an isolated nucleic acid molecule of the invention is at least 15, 20, 25, 30, 40, 60, 80, 100, 150, 200, 250, 300, 350, 400, 450, 550, 650, 700, 800, or more nucleotides in length

### VII. Treatment of Impaired Blood Glucose Levels, Impaired Blood Glucose Level Control, and Diabetes

As demonstrated herein, administration of muscle targeted Eno1 protein reduced blood glucose levels. The invention provides methods of treatment of subjects suffering from impaired glucose tolerance, increased blood glucose, insulin resistance, insulin insufficiency, and diabetes, e.g., type 2 diabetes, type 1 diabetes, pre-diabetes, and gestational diabetes by administering Eno1 to the subject to ameliorate at least one sign or symptom of the conditions. In certain embodiments, Eno1, preferably transcript variant 1 of Eno1, can be administered to a subject wherein at least one additional agent for the treatment of impaired glucose tolerance, increased blood glucose, insulin resistance, insulin insufficiency, or diabetes is administered to the subject. As used herein, the agents can be administered sequentially, in either order, or at the same time. Administration of multiple agents to a subject does not require co-formulation of the agents or the same administration regimen.

The method of treatment of impaired glucose tolerance, increased blood glucose, insulin resistance, insulin insufficiency, or diabetes, especially type 2 diabetes, using Eno1 can be combined with known methods and agents for the treatment of diabetes. Many agents and regimens are currently available for treatment of diabetes. The specific agent selected for treatment depends upon the subject, the specific symptoms and the severity of the disease state. For example, in certain embodiments, Eno1 can be administered in conjunction with dietary and/or behavior modification, e.g., caloric restriction, alone or in combination with bariatric surgery, and/or with increased physical activity. In certain embodiments, Eno1 can be administered with agents for the treatment of type 2 diabetes, e.g., metformin (Glucophage, Glumetza, others), glitazones, e.g., pioglitazone (Actos), glipizide (Glucotrol), glyburide (Diabeta, Glynase), glimepiride (Amaryl), acarbose (Precose), metformin (Glucophage), Sitagliptin (Januvia), Saxagliptin (Onglyza), Repaglinide (Prandin), Nateglinide (Starlix), Exenatide (Byetta), Liraglutide (Victoza), or insulin. Insulins are typically used only in treatment of later stage type 2 diabetes and include rapid-acting insulin (insulin aspart (NovoLog), insulin glulisine (Apidra), and insulin lispro (Humalog)); short-acting insulin (insulin regular (Humulin R, Novolin R)); intermediate-acting insulin (insulin NPH human (Humulin N, Novolin N)), and long-acting insulin (insulin glargine (Lantus) and insulin detemir (Levemir)). Treatments for diabetes can also include behavior modification including exercise and weight loss which can be facilitated by the use of drugs or surgery. Treatments for elevated blood glucose and diabetes can be combined. For example, drug therapy can be combined with behavior modification therapy. Insulins are typically used only in treatment of later stage type 2 diabetes and include rapid-acting insulin (insulin aspart (NovoLog), insulin glulisine (Apidra), and insulin lispro (Humalog)); short-acting insulin (insulin regular (Humulin R, Novolin R)); intermediate-acting insulin (insulin NPH human (Humulin N, Novolin N)), and long-acting insulin (insulin glargine (Lantus) and insulin detemir (Levemir)).

In certain embodiments, the method of treatment of impaired glucose tolerance, increased blood glucose, insulin resistance, insulin insufficiency, or diabetes, especially type 2 diabetes, using Eno1 is combined with administration of a sodium-glucose co-transporter 2 (SGLT2) inhibitor. SGLT2 facilitates glucose reabsorption in the kidney. SGLT2 inhibitors thus block the reabsorption of glucose in the kidney, increase glucose excretion, and lower blood glucose levels. In certain embodiments, the SGLT2 inhibitor is a gliflozin. Suitable gliflozins for co-administration with Eno1 include, but are not limited to, any one or more of canagliflozin, dapagliflozin, empagliflozin, ipragliflozin, tofogliflozin and ertugliflozin. In a particular embodiment, the gliflozin is ipragliflozin or ertugliflozin.

In certain embodiments, the SGLT2 inhibitor is administered at a dosage that is lower than the standard dosages of the SGLT2 inhibitor used to treat the disorder (e.g., impaired glucose tolerance, increased blood glucose, insulin resistance, insulin insufficiency, or diabetes, especially type 2 diabetes) under the standard of care for treatment for a particular disorder. Standard dosages of SGLT2 inhibitors are known to a person skilled in the art and may be obtained, for example, from the product insert provided by the manufacturer of the SGLT2 inhibitor. Examples of standard dosages of SGLT2 inhibitors are provided in Table 2 below. In certain embodiments, the dosage administered of the SGLT2 inhibitor is 5%, 10%, 20%, 30%, 40%, 50%, 60%, 70%, 80%, or 90% lower than the standard dosage of the SGLT2 inhibitor for a particular disorder (e.g. impaired glucose tolerance, increased blood glucose, insulin resistance, insulin insufficiency, or diabetes, especially type 2 diabetes). In certain embodiments, the dosage administered of the SGLT2 inhibitor is 95%, 90%, 85%, 80%, 75%, 70%, 65%, 60%, 55%, 50%, 45%, 40%, 35%, 30%, 25%, 20%, 15%, 10% or 5% of the standard dosage of the SGLT2 inhibitor for a particular disorder (e.g., impaired glucose tolerance, increased blood glucose, insulin resistance, insulin insufficiency, or diabetes, especially type 2 diabetes). In one embodiment, where a combination of SGLT2 inhibitors are administered, at least one of the SGLT2 inhibitor is administered at a dose that is lower than the standard dosage of the SGLT2 inhibitor for a particular disorder (e g, impaired glucose tolerance, increased blood glucose, insulin resistance, insulin insufficiency, or diabetes, especially type 2 diabetes). In certain embodiments, the standard dosage of the SGLT2 inhibitor is about 1, 2, 3, 4, 5, 10, 15, 20, 25, 30, 35, 40, 45, 50, 60, 70, 80, 90, 100, 125, 150, 175, 200, 225, 250, 275, 300, 325, 350, 375, 400, 425, 450, 475 or 500 mg once daily.

TABLE 2
Standard dosages of SGLT2 inhibitors. Standard dosages were obtained
from the manufacturer's product insert for the SGLT2 inhibitor.

| | |
|---|---|
| Canagliflozin | The recommended starting dose is 100 mg |
| (INVOKANA ™) | once daily, taken before the first meal of the |
| | day. |
| | Dose can be increased to 300 mg once daily |
| | in patients tolerating INVOKANA ™ 100 mg |
| | once daily who have an eGFR of 60 |
| | mL/min/1.73 m² or greater and require |
| | additional glycemic control. |
| Dapagliflozin | The recommended starting dose is 5 mg once |
| (FARXIGA ™) | daily, taken in the morning, with or without |
| | food. |
| | Dose can be increased to 10 mg once daily in |
| | patients tolerating FARXIGA ™ who require |
| | additional glycemic control. |
| Empagliflozin | The recommended dose of JARDIANCE ™ is |
| (JARDIANCE ™) | 10 mg once daily, taken in the morning, with |
| | or without food. |
| | Dose may be increased to 25 mg once daily. |

### VIII. Animal Models of Diabetes and Insulin Resistance

A number of genetic and induced animal models of metabolic syndromes such as type 1 and type 2 diabetes, insulin resistance, hyperlipidemia, are well characterized in the art. Such animals can be used to demonstrate the efficacy of Eno1 in the treatment of insulin resistance and diabetes. Models of type 1 diabetes include, but are not limited to, NOD mice and streptozotocin-induced diabetes in rats and mice (models of type 1 diabetes). Genetic and induced models of type 2 diabetes include, but are not limited to, the leptin deficient ob/ob mouse, the leptin receptor deficient db/db mouse, and high fat fed mouse or rat models. In each of the models, the timeline for development of specific disease characteristics are well known and described in the art. Eno1 can be administered before or after the appearance of symptoms of diabetes or insulin resistance to demonstrate the efficacy of Eno1 in the prevention or treatment of diabetes and/or insulin resistance in these animal models.

Depending on the specific animal model selected and the time of intervention, e.g., before or after the appearance of diabetes and/or insulin resistance, the animal models can be used to demonstrate the efficacy of the methods provide herein for the prevention, treatment, diagnosis, and monitoring of diabetes and/or insulin resistance.

For example, the results of in vivo studies in diabetic animal models discussed herein demonstrate a role for Eno1 muscle targeted fusion proteins in insulin dependent and independent glucose uptake, glucose tolerance, insulin sensitivity, and/or diabetes, e.g., type 1 diabetes, type 2 diabetes, pre-diabetes, and gestational diabetes. More specifically, administration of an Eno1 fusion protein comprising a muscle targeting peptide reduced fed blood glucose levels in a diabetic mouse model (db/db mice). Similar results are expected in other genetic models of both type 1 and type 2 diabetes.

### IX. METHODS OF TREATMENT OF OBESITY OR OVERWEIGHT

As demonstrated herein, administration of Eno1 protein reduces rosiglitazone-induced weight gain in a diabetic mouse model. Accordingly, the present invention provides, in one aspect, a method of treating obesity in a subject in need thereof, comprising administering to the subject a therapeutically effective amount of a composition of the invention, e.g., comprising an Eno1 molecule comprising an Eno1 polypeptide or a fragment thereof, thereby treating obesity in the subject.

In one embodiment the subject is obese or suffering from obesity, i.e. has a body mass index (BMI) equal to or greater than 30 kg/m². In some embodiments the subject is obese and is afflicted with diabetes, e.g. type 2 diabetes, type 1 diabetes, or pre-diabetes. In some embodiments, the subject is obese, afflicted with diabetes, and the obesity condition is exacerbated by a therapeutic treatment. In some embodiments, the therapeutic treatment is administration of a drug that induces weight gain. In some embodiments, the drug that induces weight gain is a drug for treatment of diabetes. In a particular embodiment, the diabetic drug is rosiglitazone.

In some embodiments, the subject is obese and is not afflicted with diabetes. For example, in some embodiments, the subject is not afflicted with diabetes and the obesity condition is caused or exacerbated by a therapeutic treatment, for example, administration of a drug that induces weight gain. In some embodiments, the drug that induces weight gain is not a drug for treatment of diabetes, e.g., the diabetic drug is not rosiglitazone.

In another aspect, the present invention provides a method of reducing body weight in a subject comprising administering to the subject a therapeutically effective amount of a composition of the invention, e.g., comprising an Eno1 molecule comprising an Eno1 polypeptide or a fragment thereof, thereby reducing body weight in the subject.

In some embodiments the subject is obese, i.e., has a body mass index (BMI) equal to or greater than 30 kg/m². In some embodiments, the subject in not obese, but is at risk of becoming obese. For example, in some embodiments the subject is overweight, i.e. has a body mass index (BMI) greater than or equal to 25 kg/m² and less than 30 kg/m². In some embodiments the subject is obese or overweight and is afflicted with diabetes, e.g. type 2 diabetes, type 1 diabetes, or pre-diabetes. In some embodiments, the subject is obese or overweight, afflicted with diabetes, and the obesity or overweight condition is exacerbated by a therapeutic treatment. In some embodiments, the therapeutic treatment is administration of a drug that induces weight gain. In some embodiments, the drug that induces weight gain is a drug for treatment of diabetes. In a particular embodiment, the diabetic drug is rosiglitazone.

In some embodiments, the subject is obese or overweight and is not afflicted with diabetes. For example, in some embodiments, the subject is not afflicted with diabetes and the obesity or overweight condition is caused or exacerbated by a therapeutic treatment, for example, administration of a drug that induces weight gain. In some embodiments, the drug that induces weight gain is not a drug for treatment of diabetes, e.g., the diabetic drug is not rosiglitazone.

In another aspect, the invention provides a method of reducing or preventing body weight gain in a subject, comprising administering to the subject a therapeutically effective amount of a composition of the invention, e.g., comprising an Eno1 molecule comprising an Eno1 polypeptide or a fragment thereof, thereby reducing or preventing body weight gain in the subject.

In various embodiments, the composition is administered to a subject in need of reducing or preventing body weight gain. For example, in certain embodiments the subject is at risk or increased risk for gaining body weight. For example, in certain embodiments the subject is in need of receiving a therapeutic treatment, e.g., administration of an active agent or drug, that induces, is known to induce, or has the capacity to cause weight gain. Therapeutic agents known to induce or have the capacity to cause weight gain would be recognized by one of skill in the art. For example, in some embodiments, the subject is in need of treatment with a therapeutic treatment that induces or has the capacity to cause weight gain, wherein the therapeutic treatment is selected from the group consisting of an anti-psychotic agent, an antidepressant, a mood stabilizer, an anticonvulsant, a steroid hormone, a beta-blocker, an oral contraceptive, an antihistamine, an HIV antiretroviral drug, an antihyperlipemic agents, a hypotensive or antihypertensive agent, a chemotherapeutic agent, an immunotherapeutic agent, and an immunosuppressive agent. In some embodiments, the subject is in need of treatment with a therapeutic treatment that induces or has the capacity to cause weight gain, wherein the therapeutic treatment is a diabetic drug. In other embodiments, the subject is at risk for weight gain due to changes in hormone levels, such as during premenopause or menopause in women, or due to hypothyroidism, cushing syndrome or increased cortisol (stress hormone) production. In other embodiments, the subject is at risk for weight gain because the subject is suffering from polycystic ovarian syndrome (PCOS).

In some embodiments, the subject is afflicted with a disorder selected from the group consisting of psychosis, depression, HIV, hypertension, cancer and an immune disorder. In some embodiments, the subject has any one or more of elevated blood glucose, decreased glucose tolerance, decreased insulin sensitivity and/or insulin resistance, diabetes, elevated Hb1Ac level, and abnormal blood glucose level control. In some embodiments, the subject is obese or overweight, and is at risk for further body weight gain due to any of the factors described herein.

The methods described above may further comprise selecting a patient for treatment with the composition comprising Eno1 or a fragment thereof. For example, in some embodiments, the methods further comprise selecting a subject having any one or more of obesity, overweight, elevated blood glucose, decreased glucose tolerance, decreased insulin sensitivity and/or insulin resistance, diabetes, elevated Hb1Ac level, and abnormal blood glucose level control. In some embodiments the methods further comprise selecting a subject afflicted with a disorder selected from the group consisting of psychosis, depression, HIV, hypertension, cancer and an immune disorder. In some embodiments, the methods further comprise selecting a subject at risk for weight gain. In some embodiments the methods comprise selecting a subject in need of treatment for a disorder selected from the group consisting of psychosis, depression, HIV, hypertension, cancer and an immune disorder. In some embodiments the methods further comprise selecting a subject in need of treatment for, or who is undergoing treatment for, a disorder selected from the group consisting of psychosis, depression, HIV, hypertension, cancer and an immune disorder, wherein the treatment causes or induces weight gain.

In certain embodiments, the administration of Eno1 to a subject reduces body weight in the subject relative to a control, or reduces or prevents body weight gain in the subject relative to a control. In some embodiments, the control is one or more control subjects that has not been administered Eno1. In some embodiments, the control is an average from a group or population of subjects that have not been administered Eno1, e.g., a predetermined average from said group or population. In some embodiments, the control subject has a similar clinical situation as the subject being administered Eno1. For example, in some embodiments, the subject is administered Eno1 in combination with a diabetic drug, while the control subject is administered the same diabetic drug but is not administered Eno1.

In certain embodiments of the invention, administration of Eno1 and optionally one or more additional therapeutic agents results in a reduction in BMI of at least 1%, 2%, 3%, 4%, 5%, 6%, 7%, 8%, 9%, 10%, 15%, 20%, 25%, 30%, 40%, 50%, 60%, 70% or 80% relative to a control, e.g. a subject or a population of subjects that has not been administered Eno1. In certain embodiments, administration of Eno1 and optionally one or more additional therapeutic agents results in a reduction in body weight of at least 1%, 2%, 3%, 4%, 5%, 6%, 7%, 8%, 9%, 10%, 15%, 20%, 25%, 30%, 40%, 50%, 60%, 70% or 80% relative to a control, e.g. a subject or a population of subjects that has not been administered Eno1. In certain embodiments, administration of Eno1 attenuates body weight gain by at least 1%, 2%, 3%, 4%, 5%, 6%, 7%, 8%, 9%, 10%, 15%, 20%, 25%, 30%, 40%, 50%, 60%, 70% or 80%, relative to a control, e.g. a subject or a population of subjects that has not been administered Eno1.

In certain embodiments, the subject that is administered Eno1 and optionally one or more additional therapeutic agents has a BMI of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, or 130 kg/m². Any of these values may be used to define a range for the BMI of a subject. For example the BMI of a subject may range from 25-30 kg/m², 30-40 kg/m², or 30-100 kg/m². In certain embodiments, the subject that is administered Eno1 and optionally one or more additional therapeutic agents has a BMI of at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100 kg/m².

### Combination Therapies

In one embodiment of the methods of the invention, the method further comprises administering an additional therapeutic agent, e.g., diabetes mellitus-treating agents, diabetic complication-treating agents, antihyperlipemic agents, hypotensive or antihypertensive agents, anti-obesity agents, diuretics, chemotherapeutic agents, immunotherapeutic agents and immunosuppressive agents. Eno1 and the additional therapeutic agent may act additively or synergistically. In one embodiment, Eno1 is administered concurrently with the administration of the additional therapeutic agent. In another embodiment, Eno1 is administered prior or subsequent to administration of the additional therapeutic agent.

For example, the methods of treatment of obesity, reducing body weight and preventing body weight gain using Eno1 as described herein can be combined with known methods and agents for the treatment of diabetes. Many agents and regimens are currently available for treatment of diabetes. The specific agent selected for treatment depends upon the subject, the specific symptoms and the severity of the disease state. For example, in certain embodiments, Eno1 can be administered in conjunction with dietary and/or behavior modification, e.g., caloric restriction, alone or in combination with bariatric surgery, and/or with increased physical activity. In certain embodiments, Eno1 can be administered with a diabetic drug, e.g. a drug for treatment of type 2 diabetes. Drugs for treatment of type 2 diabetes include, but are not limited to, GLP-1 (glucagon-like peptide 1) receptor agonists (e.g. GLP-1 peptide, incretin mimetics, exenatide (Byetta/Bydureon), liraglutide (Victoza, Saxenda), lixisenatide (Lyxumia), albiglutide (Tanzeum), dulaglutide (Trulicity)); meglitinides (repaglinide (Prandin/Prandimet) and nateglinide (Starlix); sulfonylureas (glipizide (Glucotrol/Metaglip), glimepiride (Amaryl/Duetact/Avandaryl), glyburide (DiaBeta, Glynase, Micronase, Glucovance), gliclazine, chloropropamide (Diabinese, tolazamide (Tolinase), and tolbutamide (Orinase, Tol-Tab)); Dipeptidy peptidase-4 (DPP-4) inhibitors (saxagliptin (Onglyza/Kombiglyze), sitagliptin (Januvia/Janumet/Juvisync), alogliptin (Nesina/Kazano/Oseni), linagliptin (Tradjenta/Glyxambi/Jentadueto)); biguanides (metformin (Fortamet, Glucophage, Riomet, Glumetza, Metformin Hydrochloride ER)); thiazolidinediones (rosiglitazone (Avandia/Avandaryl/Amaryl M) and pioglitazone (Actos/Oseni/Actoplus)); amylinomimetic drugs (pramlintide (Symlin)); dopamine agonists (bromocriptine (Parlodel, Cycloset)); sodium glucose transporter 2 (SGLT-2) inhibitors (dapagliflozin (Farxiga/Xigduo XR), canagliflozin (Ivokana/Ivokamet), empagliflozin (Jardiance/Glyxambi/Synjardy), ipraglifozin, tofogliflozin, luseoglifozin, ertugliflozin, LX 4211, EGT001442, GW 869682, and ISIS 388626); bile acid sequestrants (colesevelam hydrochloride (Welchol)); and alpha-glucosidase inhibitors (acarbose (Precose) and miglitol (Glyset)). Insulins are typically used only in treatment of later stage type 2 diabetes and include rapid-acting insulin (insulin aspart (NovoLog), insulin glulisine (Apidra), insulin lispro (Humalog), insulin inhalation powder (Afrezza)); short-acting insulin (insulin regular (Humulin R, Novolin R)); intermediate-acting insulin (insulin NPH human (Humulin N, Novolin N)), and long-acting insulin (insulin glargine (Lantus, Toujeo), insulin detemir (Levemir), and insulin degludec (Tresiba)). Agents for the treatment of diabetes are known in the art and are described, for example, in Cherney, 2016, A Complete List of Diabetes Medications, Healthline, retrieved from healthline.com/health/diabetes/medications-list; and Chao, 2014, Clinical Diabetes 32(1): 4-11, each of which is incorporated herein in its entirety. Treatments for diabetes can also include behavior modification including exercise and weight loss which can be facilitated by the use of drugs or surgery. Treatments for elevated blood glucose and diabetes can be combined. For example, drug therapy can be combined with behavior modification therapy.

In certain embodiments, Eno1 is administered with a therapeutic agent that induces weight gain in a subject. In certain embodiment, the therapeutic agent that induces weight gain is a diabetic drug. Therapeutic agents for the treatment of diabetes that induce weight gain include, but are not limited to, sulfonylureas, insulin, GLP-1 receptor agonists, DPP-4 inhibitors, metformin, rosiglitazone, pioglitazone, glyburide repaglinide and tolbutamide. In a further particular embodiment, Eno1 is administered with a GLP-1 receptor agonist and a DPP-4 inhibitor.

In certain embodiments, the therapeutic agent that induces weight gain is an antipsychotic agent. Antipsychotic agents that induce weight gain include, but are not limited to, amisulpride, aripiprazole, asenapine, blonanserin, bifeprunox, clotiapine, clozapine, iloperidone, lithium, lurasidone, mosapramine, melperone, olanzapine, paliperidone, perospirone, pimavanserin, quepin, quetiapine, remoxipride, risperidone, sertindole, sulpiride, vabicaserin, ziprasidone, and zotepine. Antipsychotic agents that induce weight gain are described for example in Vieweg et al. (2012, Focal Point: Youth, Young Adults, & Mental Health. Healthy Body—Healthy Mind, Summer, 26(1): 19-22) and US 2014/0349999, each of which is incorporated by reference herein in its entirety.

Additional therapeutic agents that induce weight gain in a subject include, but are not limited to antidepressants (e.g., citalopram (Celexa), fluoxetine (Prozac), fluvoxamine (Luvox), paroxetine (Paxil), and sertraline (Zoloft)), mood stabilizers, anticonvulsants, steroid hormones (e.g., methylprednisolone (Medrol), prednisolone (Orapred, Pediapred, Prelone), prednisone (Deltasone, Prednicot, and Sterapred), beta-blockers (e.g., acebutolol (Sectral), atenolol (Tenormin), metoprolol (Lopressor, Toprol XL), and propranolol (Inderal), oral contraceptives, antihistamines (e.g., cetirizine (Zyrtec), diphenhydramine (Benadryl), fexofenadine (Allegra), and loratadine (Claritin), HIV antiretroviral drugs, antiseizure and antimigraine drugs (e.g., amitriptyline (Elavil), nortriptyline (Aventyl, Pamelor), and valproic acid (Depacon, Depakote, Stavzor), and protease inhibitors. See 2010/0215635, which is incorporated by refrence herein in its entirey. Therapeutic agents that induce weight gain are described, for example, in Booth, 2015, Are Your Meds Making you Gain Weight?, WebMD, retrieved from webmd.com/diet/obesity/medication-weight-gain, which is incorporated herein in its entirety.

Examples of other therapeutic agents which can be used with Eno1 include, but are not limited to, diabetic complication-treating agents, antihyperlipemic agents, hypotensive or antihypertensive agents, anti-obesity agents, diuretics, chemotherapeutic agents, immunotherapeutic agents, immunosuppressive agents, and the like.

Examples of agents for treating diabetic complications include, but are not limited to, aldose reductase inhibitors (e.g., tolrestat, epalrestat, zenarestat, zopolrestat, minalrestat, fidareatat, SK-860, CT-112 and the like), neurotrophic factors (e.g., NGF, NT-3, BDNF and the like), PKC inhibitors (e.g., LY-333531 and the like), advanced glycation end-product (AGE) inhibitors (e.g., ALT946, pimagedine, pyradoxamine, phenacylthiazolium bromide (ALT766) and the like), active oxygen quenching agents (e.g., thioctic acid or derivative thereof, a bioflavonoid including flavones, isoflavones, flavonones, procyanidins, anthocyanidins, pycnogenol, lutein, lycopene, vitamins E, coenzymes Q, and the like), cerebrovascular dilating agents (e.g., tiapride, mexiletene and the like).

Antihyperlipemic agents include, for example, statin-based compounds which are cholesterol synthesis inhibitors (e.g., pravastatin, simvastatin, lovastatin, atorvastatin, fluvastatin, rosuvastatin and the like), squalene synthetase inhibitors or fibrate compounds having a triglyceride-lowering effect (e.g., fenofibrate, gemfibrozil, bezafibrate, clofibrate, sinfibrate, clinofibrate and the like).

Hypotensive agents include, for example, angiotensin converting enzyme inhibitors (e.g., captopril, enalapril, delapril, benazepril, cilazapril, enalapril, enalaprilat, fosinopril, lisinopril, moexipril, perindopril, quinapril, ramipril, trandolapril and the like) or angiotensin II antagonists (e.g., losartan, candesartan cilexetil, olmesartan medoxomil, eprosartan, valsartan, telmisartan, irbesartan, tasosartan, pomisartan, ripisartan forasartan, and the like).

Antiobesity agents include, for example, central antiobesity agents (e.g., dexfenfluramine, fenfluramine, phentermine, sibutramine, amfepramone, dexamphetamine, mazindol, phenylpropanolamine, clobenzorex and the like), gastrointestinal lipase inhibitors (e.g., orlistat and the like), 13-3 agonists (e.g., CL-316243, SR-58611-A, UL-TG-307, SB-226552, AJ-9677, BMS-196085 and the like), peptide-based appetite-suppressing agents (e.g., leptin, CNTF and the like), cholecystokinin agonists (e.g., lintitript, FPL-15849 and the like), serotonin 2C receptor agonists (e.g., lorcaserin (Belviq)), monoamine reuptake inhibitors (e.g., tesofensine), and the like. Antiobesity agents can also include drug combinations, including combinations of opiod antagonists (naltrexone) and antidepressants (buproprion), such as Contrave; combinations of phentermine and antiseizure agents (topiramate), such as Qsymia; combinations of antidepressants (buproprion) and antiseizure agents (zonsiamide), such as Empatic. See Adan, 2013, Trends Neurosci., 36(2): 133-40; Gustafson et al., 2013, P. T., 38(9): 525-34; Shin and Gadde, 2013, Diabetes Metab. Syndr. Obes., 6: 131-9; Bello and Zahner, 2009, Curr. Opin. Investig. Drugs, 10(10) 1105-16, each of which is incorporated herein in its entirety.

This invention is further illustrated by the following examples which should not be construed as limiting. The contents of all references and published patents and patent applications cited throughout the application are hereby incorporated by reference.

### EXAMPLES

### Example 1

### Expression, Purification and Characterization of Native Eno1 and an Eno1 Fusion Protein

Native human Eno1 (enolase α), an Eno1 fusion protein comprising an N-terminal muscle targeting peptide (MTP) (ASSLNIA) (SEQ ID NO: 7) and a protease tag (SSGVDLGTENLYFQ) (SEQ ID NO: 6), and human Eno1 with the N-terminal methionine removed (SEQ ID NO: 13) were each recombinantly expressed in E. coli strain BL21 (DE3) using a pJExpress401 bacterial expression vector. The native Eno1 contains several reduced cysteine residues and is not N-glycosylated. The amino acid sequence of the Eno1 fusion protein is shown below. The N-terminal methionine, MTP and protease tag are underlined.

(SEQ ID NO: 5)
MASSLNIASSGVDLGTENLYFQSILKIHAREIFDSRGNPTVEVDLF
TSKGLFRAAVPSGASTGIYEALELRDNDKTRYMGKGVSKAVEHINK
TIAPALVSKKLNVTEQEKIDKLMIEMDGTENKSKFGANAILGVSLA
VCKAGAVEKGVPLYRHIADLAGNSEVILPVPAFNVINGGSHAGNKL
AMQEFMILPVGAANFREAMRIGAEVYHNLKNVIKEKYGKDATNVGD
EGGFAPNILENKEGLELLKTAIGKAGYTDKVVIGMDVAASEFFRSG
KYDLDFKSPDDPSRYISPDQLADLYKSFIKDYPVVSIEDPFDQDDW
GAWQKFTASAGIQVVGDDLTVTNPKRIAKAVNEKSCNCLLLKVNQI
GSVTESLQACKLAQANGWGVMVSHRSGETEDTFIADLVVGLCTGQI
KTGAPCRSERLAKYNQLLRIEEELGSKAKFAGRNFRNPLAK

The bacteria were grown in a 12 liter shake flask in Terrific Broth medium, and expression of the fusion protein was induced with 1 mM IPTG at 37° C. The bacterial cultures were centrifuged to form a cell pellet and the supernatant was removed. The cell pellet was disrupted using a microfluidizer, and the Eno1 proteins were isolated from the soluble fraction DEAE Sephacel single column purification. This process yields up to 1 g of Eno1 protein per 12 liter shake flask. Fermentations were run in several 12 liter shake flasks, and the protein was purified and pooled.

The native Eno1 and Eno1 fusion proteins were formulated in PBS buffer with or without 100 mM MgCl₂. The presence of excess Mg²⁺ appears to be important for maintaining monomer-monomer association. The proteins were also formulated in an alternate formulation of 50 mM Tris; pH 8.0, 20 mM MgSO₄, 150 mM NaCl, 2 mM DTT, and 10% glycerol.

At least 40 mg/ml of the fusion protein could be dissolved in PBS without precipitation. An upper limit for solubility of the fusion protein was not determined

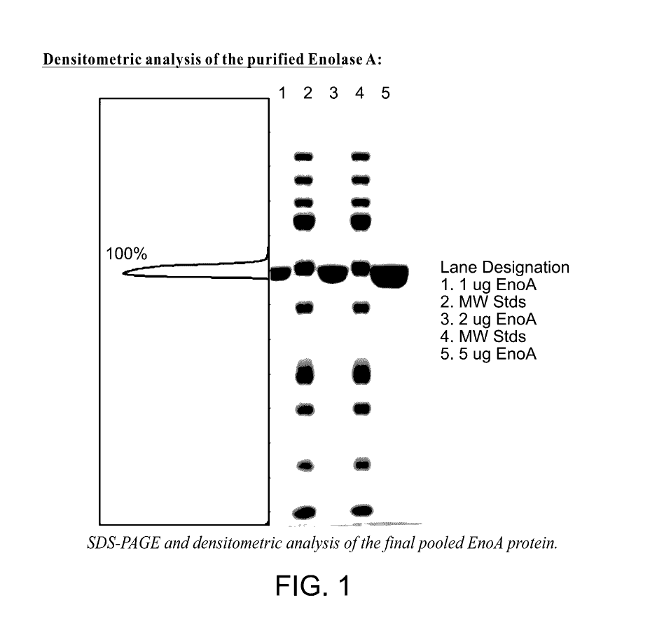

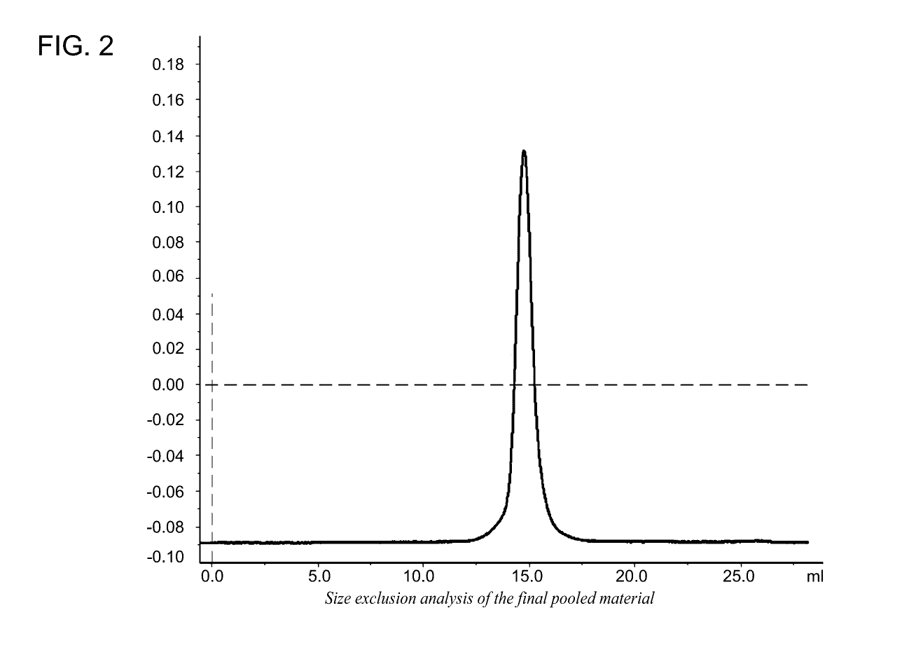

SDS-PAGE and Densitometric analysis of the native Eno1 protein indicated that the protein was greater than 99% pure and had very low to undetectable levels of endotoxin. See FIG. 1. Size exclusion analysis resulted in a single uniform peak of the final pooled native Eno1 protein, indicating the purity of the protein. See FIG. 2.

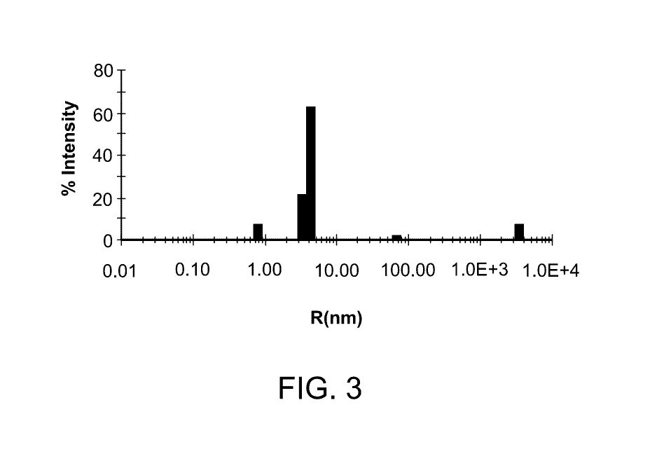

The Eno1 fusion protein was analyzed by dynamic light scattering to estimate the globular size of the protein. Dynamic light scattering analysis of the Eno1 fusion protein in PBS buffer (pH 7.4) produced a value of 4.1 nm (see FIG. 3), which is within the range of the expected value.

The native Eno1 protein and Eno1 fusion protein were further analyzed by Differential Scanning calorimetry (DSC) to measure the glass transition temperature (Tm) of the proteins, i.e. the temperature at which the proteins lose their tertiary structure. DSC analysis in PBS buffer (pH 7.4) resulted in a Tm of 55.3° C. for the Eno1 fusion protein and 48° C. for native Eno1.

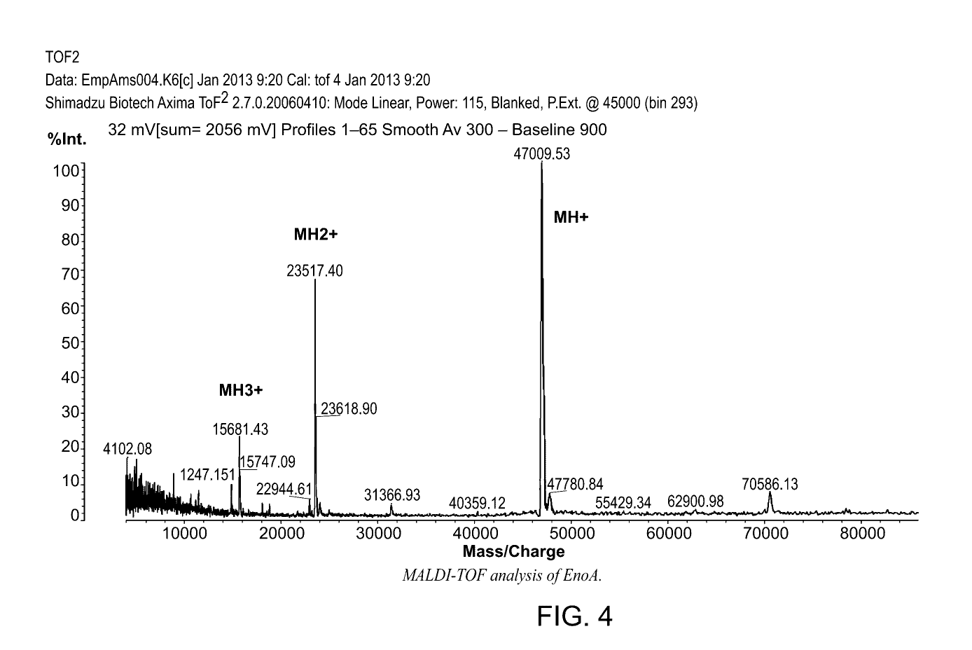

MALDI TOF analysis of the native Eno1 protein produced a primary peak (MH+) at 47,009 Da, an MH2+ peak at 23,517.4 Da, and an MH3+ peak at 15,681.4 Da. See FIG. 4. This molecular weight matches that of untagged human Eno1 in which the N-terminal methionine residue has been removed during expression, which is often the case for proteins expressed in E. coli.

Stability of the native Eno1 protein in PBS buffer was also determined Some degradation of the protein was observed by SDS-PAGE after 14 days of storage at 25° C. Native Eno1 protein stored at 4° C. for 14 days showed no evidence of degradation or precipitation. Analysis by anion exchange chromatography (AEC), thermal shift foot print, specific enzyme activity, mass spec and SDS-PAGE indicated that freezing and thawing of samples did not result in significant differences in protein stability.

The activity of the native Eno1 protein and the Eno1 fusion protein were measured using the ENO1 activity assay kit from Abcam (Catalog#117994). Protein content of the samples was measured using the Pierce BCA kit (Thermo Scientific, Catalog#23227) following the manufacturer's protocol. Three different concentrations of proteins were prepared: 500 ng/ml, 250 ng/ml, and 125 ng/ml.

10 μL of diluted sample were added to each well in a microtiter plate in triplicate, such that the total amount of protein in each well was 5 ng, 2.5 ng or 1.25 ng. The same volume of incubation buffer was also loaded for background subtraction during kinetics measurements. 200 μL of 1× activity solution was added to the wells and the plate was read immediately at 340 nm on a plate reader for kinetics for 10-15 min at intervals of 1 minute. The samples were analyzed for Eno1 activity using linear slopes. The activity assays indicated that purified native Eno1 has a specific activity comparable to the published value on a unit/mg basis. In addition, no significant difference in specific activity was observed between native Eno1 and the Eno1 fusion protein.

### Example 2

### Effect of the Eno1 Fusion Protein Administered by IV or IP Injection on Fed Blood Glucose Levels in a Genetic Model of Obesity, Db/Db Mice

A series of studies were conducted to evaluate the effect of various dosages of the Eno1 fusion protein described above in Example 1 on fed blood glucose levels in db/db mice.

Study 1. Dosage: 400 or 800 μg/kg/day

Male db/db mice (BKS.Cg-m+/+Lepr^{db}/J) mice were obtained from a commercial vendor. All mice were housed 2-3 per cage at 22° C. on a 12:12 hr day-night cycle and were acclimated for 3 weeks at animal facility on a standard chow diet. At 8 weeks of age, the following treatments were administered by intravenous injections into the tail vein twice daily at 12 hour intervals.

The treatment groups were as follows from Day 1 to Day 14 of the study:
1. saline injection (control)
2. MTP/Protease tag/Eno1 fusion protein (SEQ ID NO: 5; described in Example 1) at 400 μg/kg/day

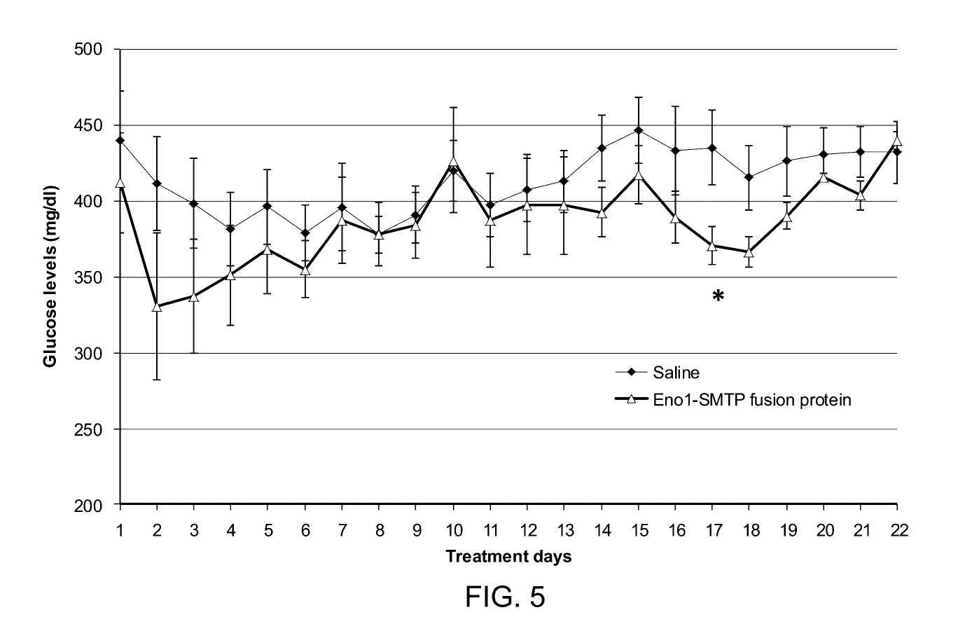

From Day 14 to Day 22, the dose of the Eno1 fusion protein was increased to 800 μg/kg/day. Fed blood glucose was measured once daily immediately before the morning injection, i.e. approximately 12 hours after the previous evening injection. As shown in FIG. 5, administration of the Eno1 fusion protein decreased fed blood glucose levels in db/db mice, with a statistically significant difference at Day 17.

At Day 22 of the study, the amount of Eno1 was measured by ELISA in serum, muscle, liver and kidney of both control and fusion protein treated mice. ELISA background levels were subtracted. The ELISA was performed with a polyclonal anti-Eno1 antibody from Novus Biologicals (Catalog No. NB100-65252) as described below.

Sample Preparation:

For muscle, kidney and liver tissue, the tissues were ground into smaller pieces in a liquid nitrogen cooled mortar and pestle. Approximately 25-50 mg tissue was homogenized in 150-200 μL of RIPA buffer containing protease and phosphatase inhibitors with stainless steel beads using an Omini blender (CLIA lab) from BBD for 2×45 seconds (lx 45 seconds for liver tissue). The volume was brought up with RIPA buffer containing inhibitors to 400 ul (depending on the tissue amount and desired final concentration). The samples were shaken on an orbital shaker for 1 hour @ RT. The samples were then spun at 14,000 g for 10 min at RT. The supernatant was taken and BCA assay was performed on the samples to measure the concentration of proteins present in each of them. The samples were then diluted 1:1 with 1× Cell Extraction Buffer PTR. The total protein concentration of the samples was reduced to 200 μg/mL for hind quarter muscle, and 750 μg/mL for kidney and liver.

For the serum samples, 5 μL of serum was added to a total of 50 μL of 1× Cell Extraction Buffer PTR.

ELISA:

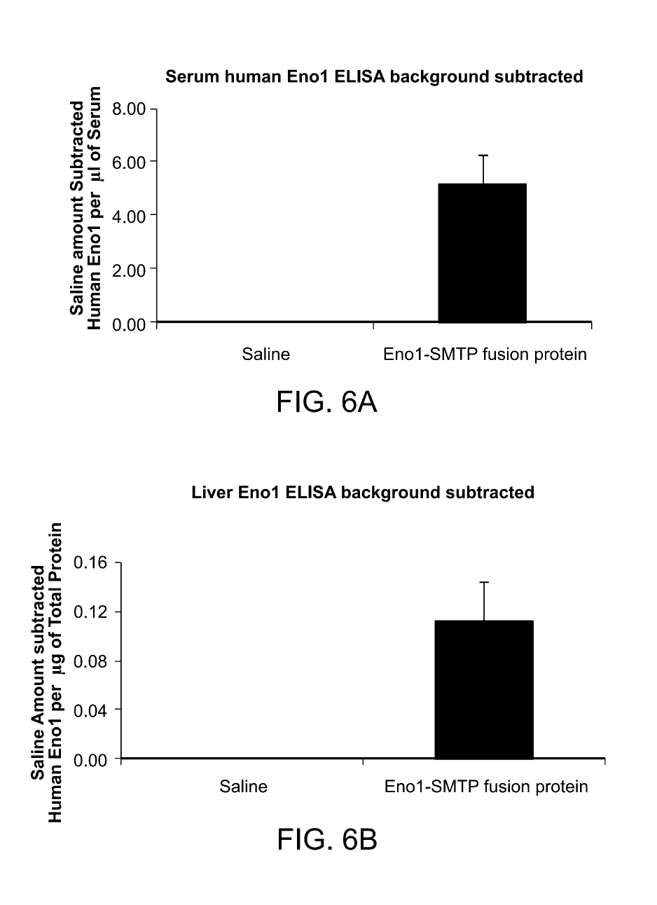

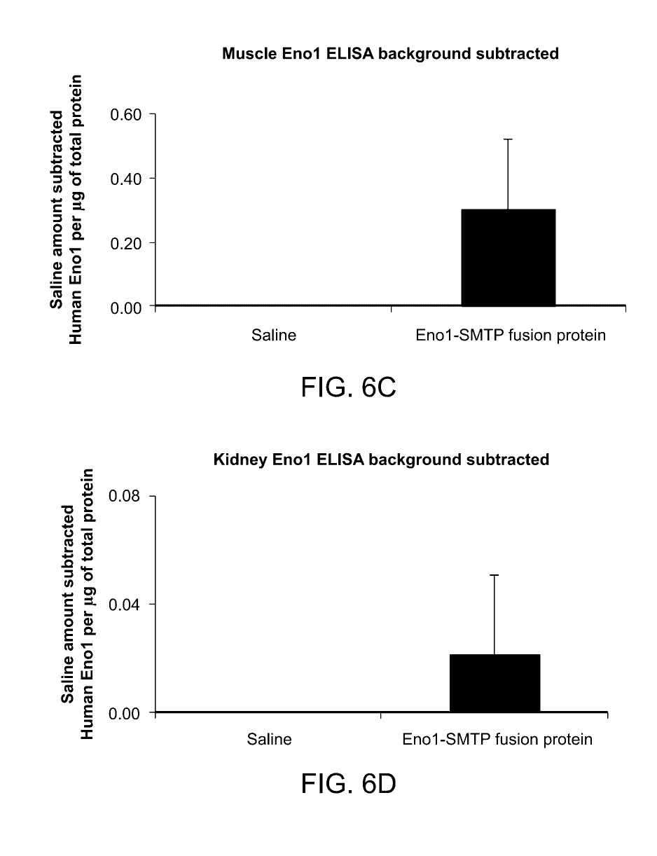

50 μL of sample or standard and 50 μL of antibody cocktail were added to the wells of a 96-well plate. Plates were sealed and incubated for 1 hour at room temperature on a plate shaker set to 400 rpm. Each well was washed with 3×350 μL 1× Wash Buffer PT by aspirating or decanting from wells and then dispensing 350 μL 1× Wash Buffer PT into each well. After the last wash the plate was inverted and blotted against clean paper towels to remove excess liquid. 100 μL of TMB substrate was added to each well and incubated for 10 minutes in the dark on a plate shaker set to 400 rpm. 100 μL of Stop Solution was added to each well and the plate was shaken on a plate shaker for 1 minute to mix. The OD at 450 nm was measured. The level of Eno1 detected in the saline-treated mice was used to indicate the background level of endogenous Eno1 expression, and this value was subtracted from the levels observed in the mice treated with the Eno1 fusion protein. The levels of Eno1 detected by ELISA with the background level subtracted are shown in FIGS. 6A-6D. Eno1 protein levels were higher in serum, muscle, liver and kidney of the mice treated with the Eno1 fusion protein.

Study 2. Dosage: 0.4 or 1.6 mg/kg/day

In a further study to evaluate the effects of intraperitoneal (IP) injection and higher doses of the Eno1 fusion protein, eight-week-old male db/db mice (BKS.Cg-m+/+Lepr^{db}/J) mice were obtained from a commercial vendor and housed and fed as described above. The mice were acclimated for 4 weeks. At 12 weeks of age, the following treatments were administered once daily for three days by intravenous (IV) injection into the tail vein or by intraperitoneal injection (IP) as indicated. Each treatment group contained three mice. The MTP/Protease tag/Eno1 fusion protein is described in Example 1 above, and the amino acid sequence is provided in SEQ ID NO: 5. The treatment groups were as follows:
1. saline (control), IV injection;
2. MTP/Protease tag/Eno1 fusion protein, 0.4 mg/kg/day, IV injection;
3. MTP/Protease tag/Eno1 fusion protein, 1.6 mg/kg/day, IV injection;
4. saline (control), IP injection; and
5. MTP/Protease tag/Eno1 fusion protein, 1.6 mg/kg/day, IP injection

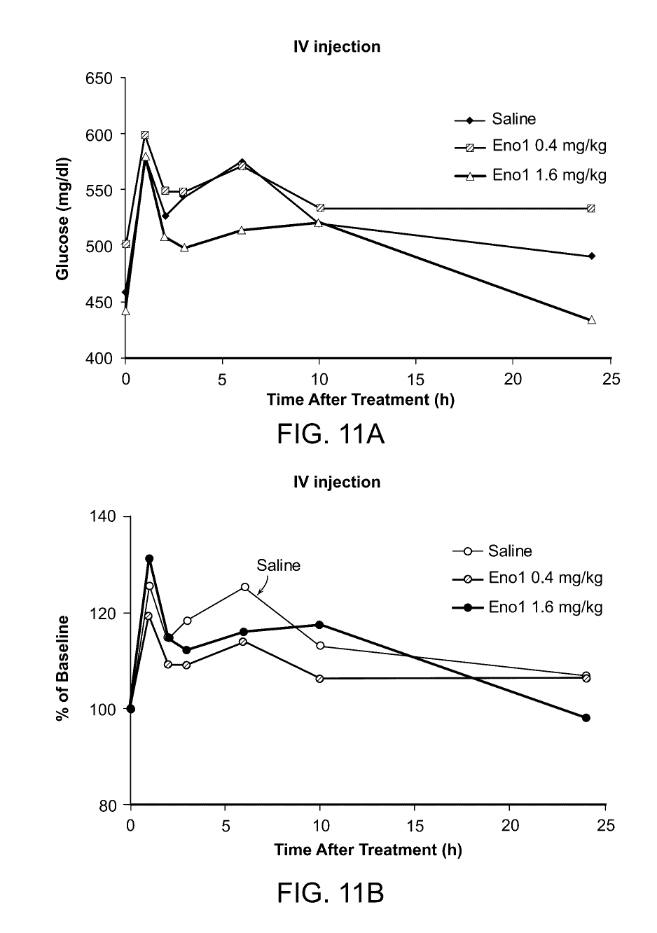

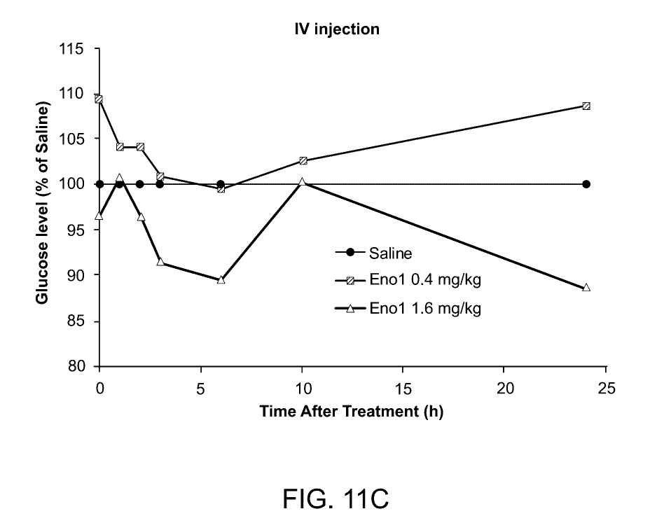

Fed blood glucose was measured immediately before the injection on the third day and 1, 2, 4, 6, 10 and 24 hours after the injection on the third day. Glucose levels were averaged over the three mice in each treatment group as shown in FIG. 11A. FIG. 11B shows glucose levels as a percentage of the initial value before Eno1 injection on the third day (% of Baseline). FIG. 11C shows glucose levels as a percentage of the saline control (% of Saline). As shown in FIG. 11A-11C, the 1.6 mg/kg/day IV dose of the Eno1 fusion protein decreased fed blood glucose levels in db/db mice relative to the saline IV control. As shown in FIG. 11B, the 0.4 mg/kg/day IV dose of the Eno1 fusion protein also decreased fed blood glucose levels relative to the saline IV control.

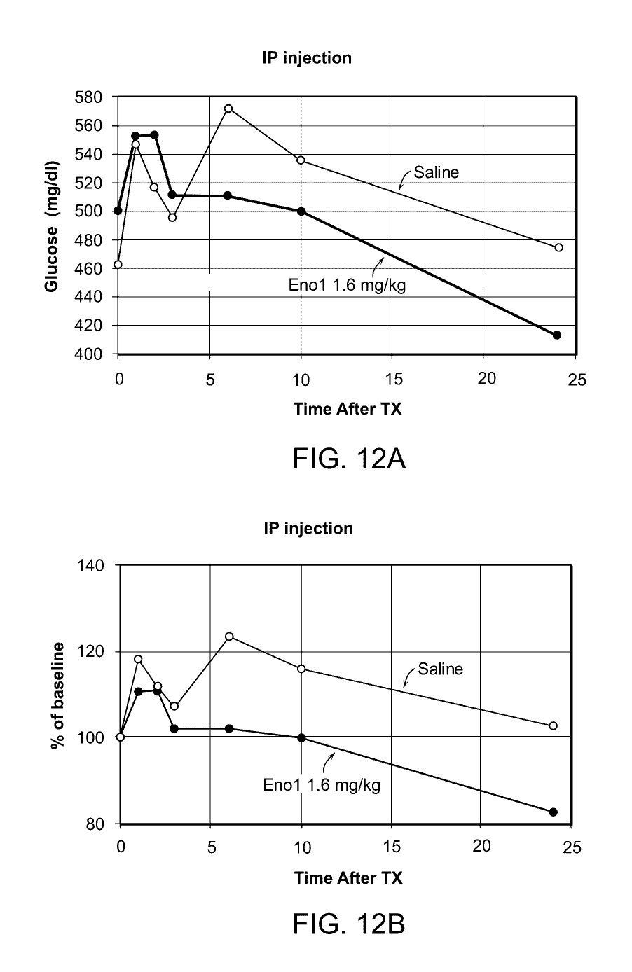

As shown in FIGS. 12A and 12B, intraperitoneal injection of 1.6 mg/kg/day of the Eno1 fusion protein also decreased fed blood glucose levels relative to the saline IP control.

Study 3. Dosage: 100, 200, 400, 600, 800 or 1200 μg/kg/day

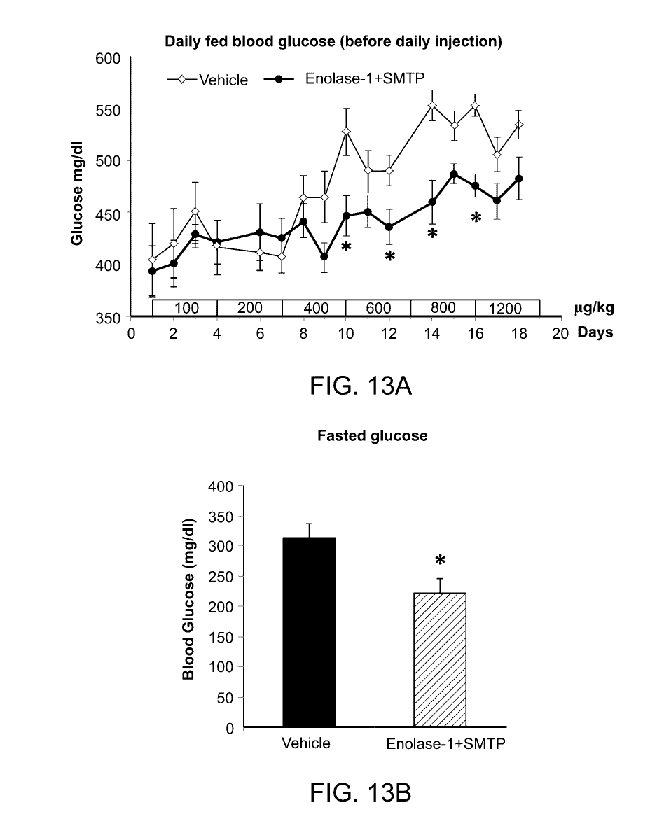

In a further dose escalation study, male db/db mice (BKS.Cg-m+/+Lepr^{db}/J) were obtained from a commercial vendor and housed and fed as described above. At 8 weeks of age, the Eno1 fusion protein described in Example 1 above or a saline control was administered twice daily by intraperitoneal injection (IP). The initial dosage of Eno1 fusion protein was 100 μg/kg/day (Days 1-3), and the dose was escalated every three days to 200 μg/kg/day (Days 4-6), 400 μg/kg/day (Days 7-9), 600 μg/kg/day (Days 10-12), 800 μg/kg/day (Days 13-15), 1200 μg/kg/day (Days 16-18) and 1600 μg/kg/day (Days 19-21, data not shown). Fed blood glucose was measured once daily immediately before the morning injection, i.e. approximately 12 hours after the previous evening injection. As shown in FIG. 13A, administration of the Eno1 fusion protein decreased fed blood glucose levels in db/db mice, with a statistically significant difference at Days 10 (400 μg/kg/day), 12 (600 μg/kg/day), 14 (800 μg/kg/day) and 16 (800 μg/kg/day).

Fasted blood glucose was also measured on the last day of the study. Mice were fasted for 12 hours. As shown in FIG. 13B, administration of the Eno1 fusion protein significantly decreased fasted blood glucose levels.

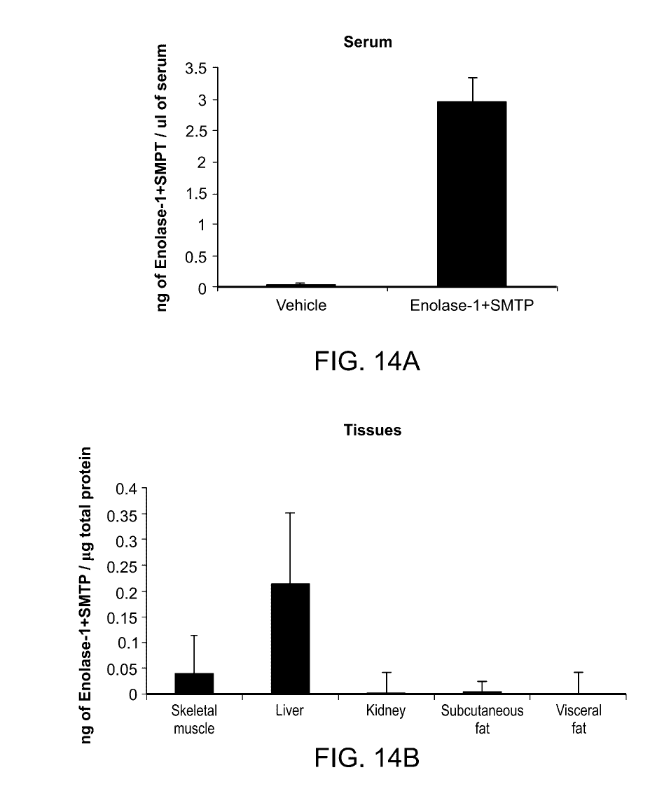

At the end of the study, the amount of Eno1 was measured by ELISA in serum, skeletal muscle, liver, kidney, subcutaneous fat and visceral fat of both control and fusion protein treated mice as described above in Study 1. Eno1 protein levels were higher in serum, skeletal muscle and liver of the mice treated with the Eno1 fusion protein, indicating that there was preferential delivery of the Eno1 fusion protein to skeletal muscle and liver. See FIGS. 14A and 14B.

### Example 3

### Production of Eno1 Proteins with Added Cysteine Residues

Several Eno1 proteins comprising added cysteine residues at various locations are produced by expression in E. coli as described above in Example 1.

Two types of variants are produced. The first type of variant contains an added cysteine residue at the N-terminus followed by a glycine/serine linker region which is attached to the N-terminus of the Eno1 protein (e.g. C-Glycine/Serine Linker-Eno1). The N-terminal added cysteine residue serves as a scaffold protein attachment site for additional functional moieties such as targeting peptides or cell penetrating peptides. In the second type of variant, serine and/or threonine residues in an Eno1 fusion protein comprising an MTP are replaced with cysteine to provide reactive sites that enable defined chemistry, for example for attaching functional moieties such as cell penetrating peptides or additional targeting groups.

Serine and threonine residues were selected for substitution because they are chemically similar to cysteine and thus are potentially less disruptive to protein structure and function. Selection of the serine and threonine residues for substitution was based on the crystal structure of human Eno1 (PDB ID: 3B97; available at ncbi.nlm.nih.gov/Structure/mmdb/mmdbsrv.cgi?uid=66725). Serine and threonine residues with 100% solvent exposed R chains were selected. Residues in active enzyme cleft locations were avoided. Seven serine residues were identified with the above characteristics: S26, S78, S140, S253, S267, S236 and S418 (numbering is based on the human Eno1 sequence with the N-terminal methionine removed, SEQ ID NO: 13).

Orientation of the three dimensional model of the Eno1 dimer along the axis of symmetry with the N-terminus at the top (see FIG. 7) reveals that positions S26 and S78 are at the top of the dimer, S140 and S418 are at the side of the dimer (near C-terminus) and S236, S253 and S267 are at the bottom. In addition, the crystal structure reveals that sites near the N-terminus (i.e. the top of the dimer) point in the same direction (up), sites at the middle point in opposite directions, and sites at the bottom point in the same direction (down). See FIG. 7. In some cases, it may be optimal to have all of the functional peptides situated facing in the same direction to capture cooperative (avidity) effects. However, in other cases closely situated peptides may self assemble and become inactive. In addition, for some peptides such as cell penetrating peptides or targeting peptides, it may be beneficial to attach several peptides to the dimer to improve cell penetration or targeting. Therefore several variants are evaluated with different numbers and positions of substitutions, i.e. some with substitutions at only the top, side or bottom of the dimer, and others with substitutions at different positions along the surface of the dimer.

The following variants are produced. The location of the residues (top, side or bottom) is shown in parentheses next to the position numbers.

### 1. C-(GGSGGSGGSGGSGGS (SEQ ID NO: 14))-Eno1

2. SMTP-Eno1 (S26C, S78C) (top, top)
3. SMTP-Eno1 (S26C, S418C, S267C) (top, side, bottom)
4. SMTP-Eno1 (5140C, S418C, S267C) (side, side, bottom)
5. SMTP-Eno1 (S236C, S253C, S267C) (bottom, bottom, bottom)
6. SMTP-Eno1 (S140C, S418C) (side, side)
7. SMTP-Eno1 (S236C, S253C, S267C) (side, bottom, bottom)

The variants are evaluated for their effects on blood glucose levels in mouse models of diabetes (e.g db/db mice or diet-induced obesity (DIO) mice) as described in Example 2.

### Example 4

### Conjugation of Creatine Analogs to Cysteine Modified Eno1

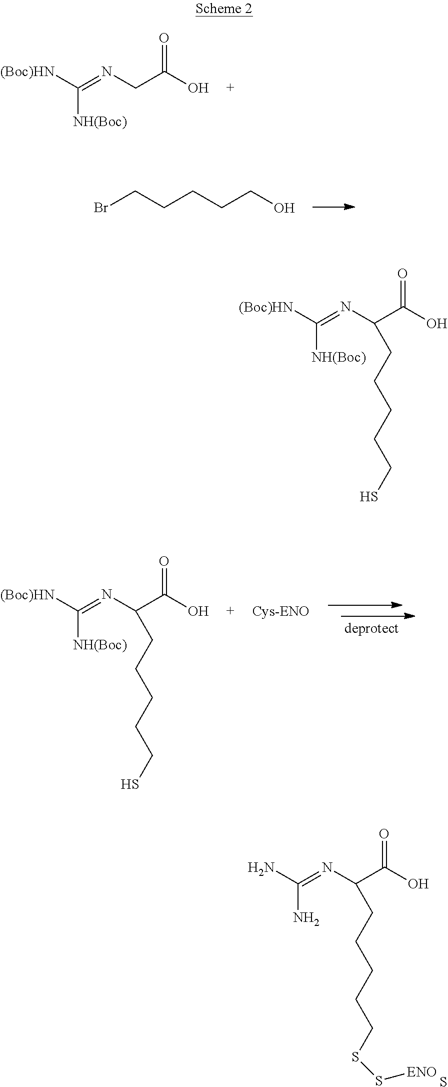

In order to conjugate a creatine analog via a disulfide linkage to a cysteine modified Eno1, a 6-carbon chain thiol containing a creatine analog is prepared. See Scheme 2. Briefly, guanidine acetic acid is reacted with bromohexanethiol in the presence of a base to yield a thiol containing creatine group which upon reaction with cysteine modified Eno1, followed by deprotection, yields a disulfide linked creatine-Eno1 conjugate.

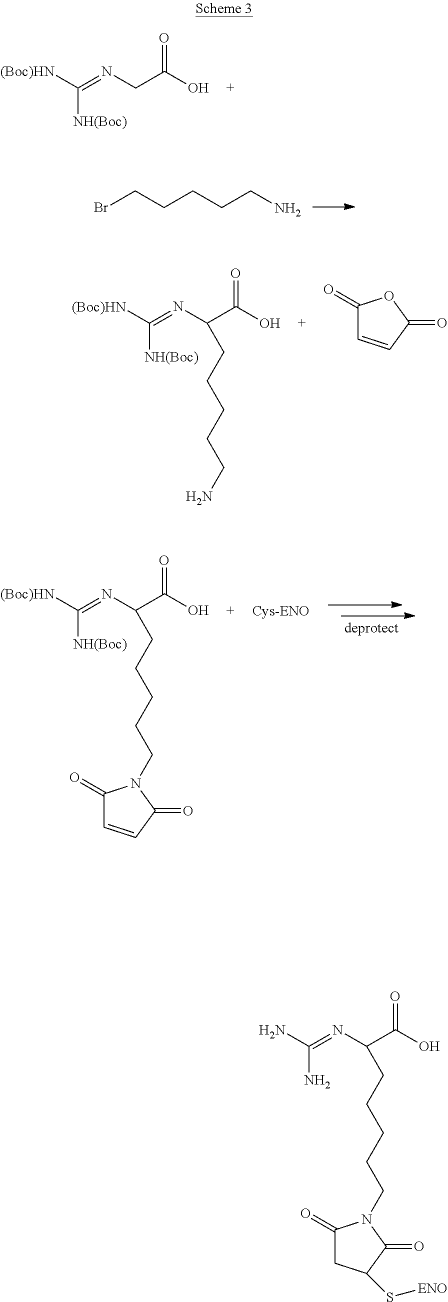

In order to conjugate a creatine analog via a thioether linkage with a cysteine modified Eno1, a 6-carbon chain maleimide containing creatine analog is prepared as shown in Scheme 3. Briefly, guanidine acetic acid is reacted with bromohexylamine in the presence of a base to yield a thiol containing creatine group, which is reacted with maleic anhydride to yield maleimide creatine. Maleimide creatine is reacted with cysteine modified Eno1, followed by deprotection, to yield a thioether linked creatine-Eno1 conjugate.

### Example 5

### Conjugation of PEG to Cysteine Modified Eno1

Cysteine modified variants of the Eno1 fusion protein described in Example 1 (SEQ ID NO: 5) were produced by expression in E. coli as described in Example 1. As discussed above, the Eno1 fusion protein comprises an N-terminal muscle targeting peptide (MTP) (SEQ ID NO: 7), a protease tag (SEQ ID NO: 6), and human Eno1 with the N-terminal methionine removed (SEQ ID NO: 13). The cysteine modified variants also comprise the peptide GIEGR (SEQ ID NO: 16) added to the C-terminus of the Eno1 protein. Four cysteine modified variants were produced in which one or more serine residues at positions 140, 267 and/or 418 of SEQ ID NO: 13 were replaced with a cysteine residue as shown below:

### 1. 5140C (SEQ ID NO: 17)

### 2. S267C (SEQ ID NO: 16)

### 3. S418C (SEQ ID NO: 18)

### 4. S140C/S267C/S418C (SEQ ID NO: 19)

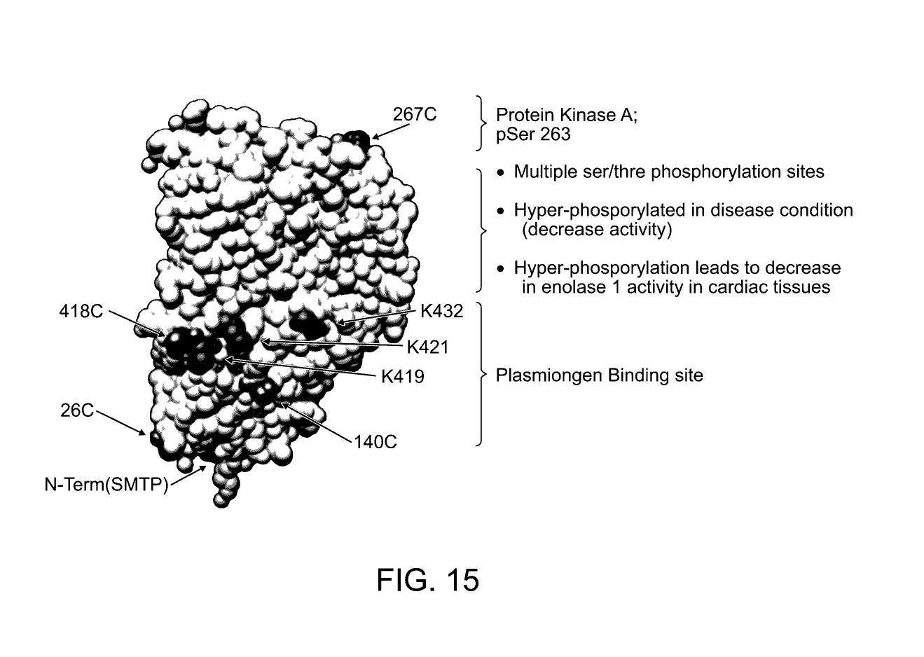

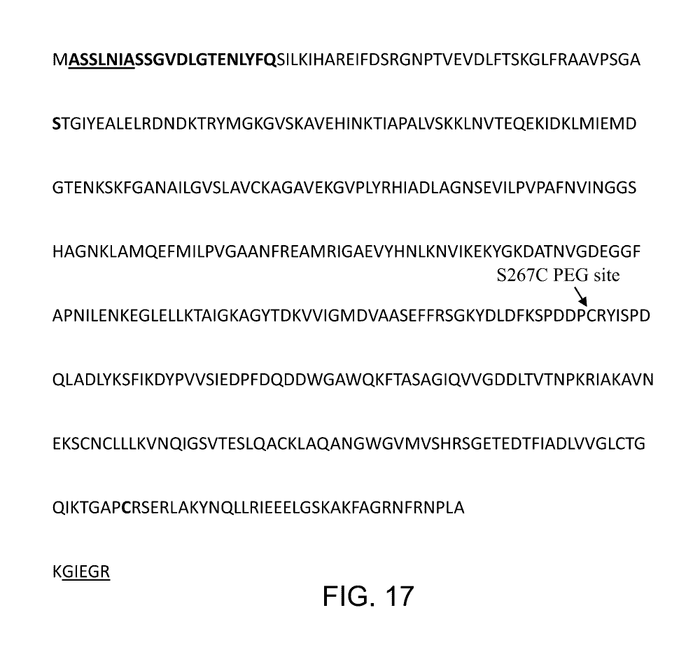

The positions of the modified residues in the three-dimensional structure of monomeric Eno1 is shown in FIG. 15. The amino acid sequence of the S267C variant (SEQ ID NO: 16) is shown in FIG. 17.

Linear 20 kDa PEG was conjugated to each cysteine modified variant using a maleimide linkage. See Scheme 1. Briefly, the cysteine modified variants of the Eno1 fusion proteins in TRIS buffer were dialyzed in 10×PBS (with Mg, pH 7.4) in the presence of nitrogen. A 10 mole excess of TCEP was added, and then a 3 mole excess of linear 20 kDa PEG-maleimide was added. The concentration of the protein was 5 mg/mL in 10×PBS. The pH was maintained at all times between 6.7 and 7.2. The reaction was gently shaken for 8 hrs at 5° C. The reduction of the unconjugated fusion protein and increase in the PEGylated fusion protein was monitored by HPLC. The addition of a 3 mole excess of linear 20 kDa PEG-maleimide was repeated one or two more times as necessary.

### Example 6

### Effect of PEGylated Cysteine Modified Eno1 on Fed Blood Glucose Levels in db/db Mice

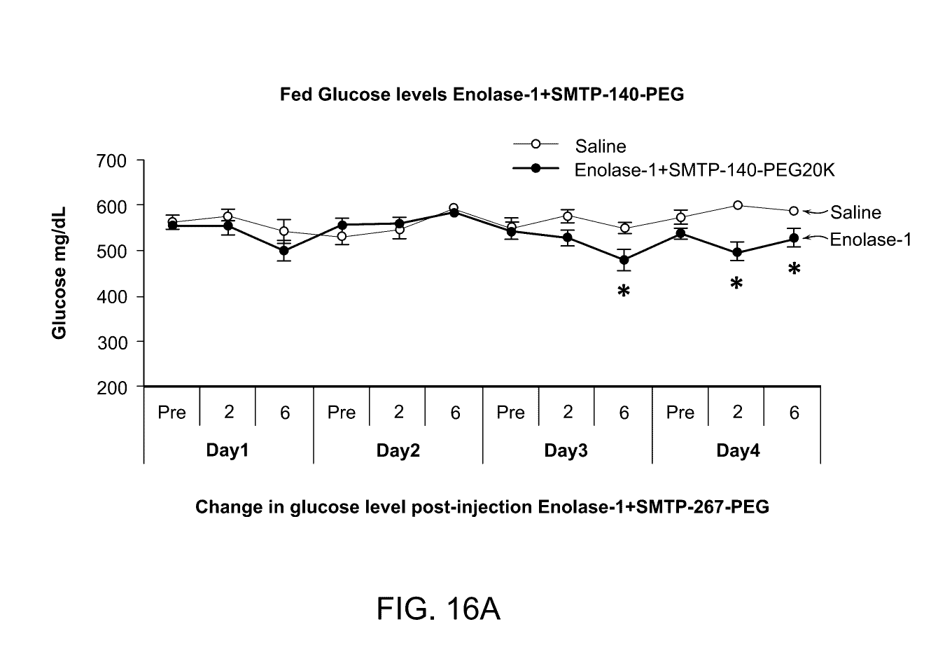

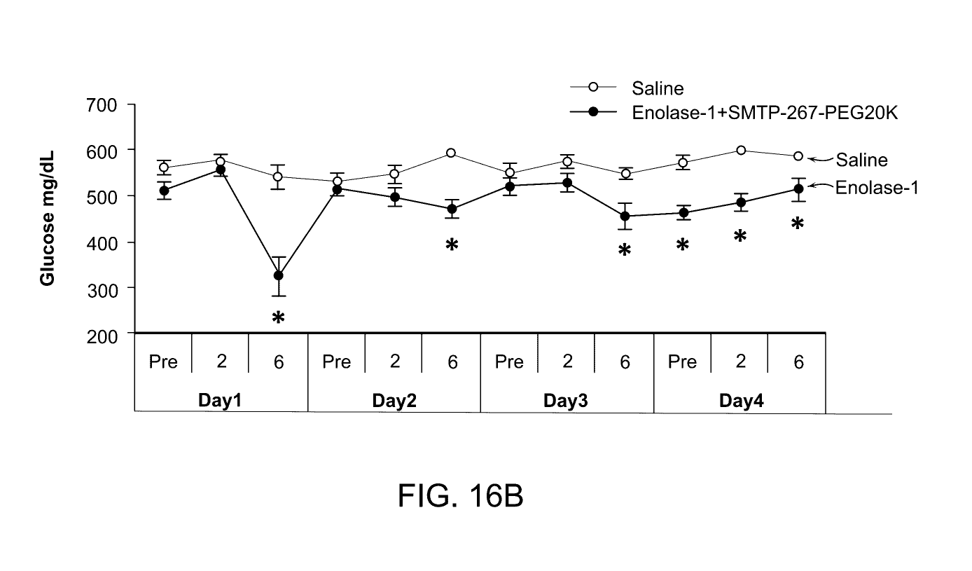

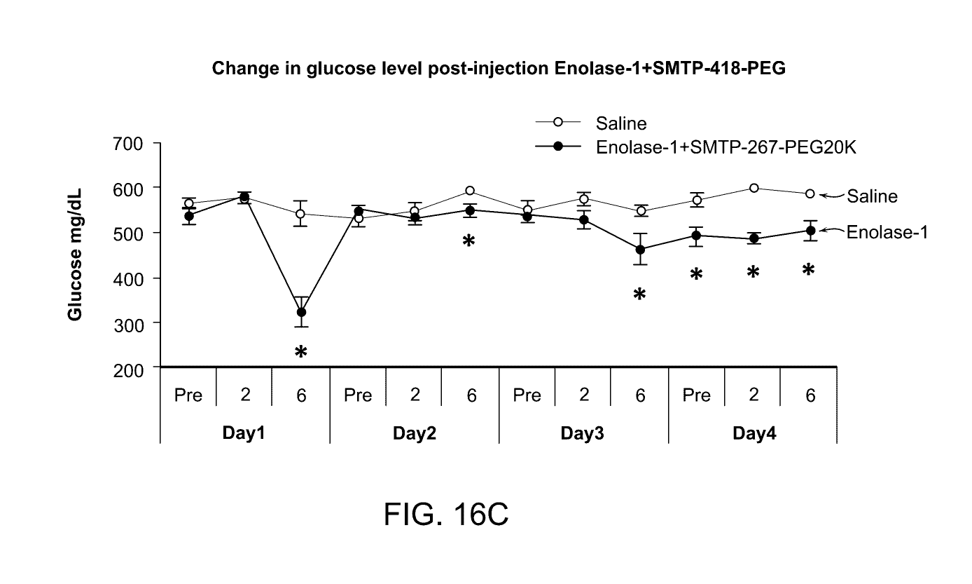

Male db/db mice (BKS.Cg-m+/+ Leprdb/J) were obtained from a commercial vendor and housed and fed as described above in Example 2. At 8 weeks of age, 1.6 mg/kg/day of each of the cysteine modified Eno1 variants described in Example 5 above was administered intravenously to the mice once daily for 4 days. Saline was administered intravenously to the mice as a negative control. Fed blood glucose levels were measured three times daily: before injection, and at 2 and 6 hours after injection. As shown in FIG. 16, each of the cysteine modified Eno1 variants S140C, S267C and S418C significantly reduced fed blood glucose levels in the db/db mice relative to the saline control.

### Example 7

### Reduction of Weight Gain by Treatment with Muscle Targeted Eno1/Dendrimer Complex and Rosiglitazone in a Genetic Model of Obesity, Db/Db Mice

A muscle targeted Eno1/dendrimer complex was generated to analyze its efficacy in reducing weight gain. The dendrimer complex comprised human Eno1, transcript variant 1 protein (SEQ ID NO: 2) which was non-covalently linked to a G5-PAMAM dendrimer/muscle targeting peptide (MTP) (ASSLNIA; SEQ ID NO: 7) conjugate. Stock solutions of Eno1 were prepared in buffer and the protein solution was mixed with the G5 dendrimer-MTP conjugate.

Lean mice and male obese and diabetic db/db mice (male BKS.Cg-m+/+Lepr^{db}/J) mice were obtained from a commercial vendor. All mice were housed 2-3 per cage at 22° C. on a 12:12 hr day-night cycle and were acclimated for 3 weeks at animal facility on a standard chow diet. At 8 weeks of age, 200 μg/kg body weight Eno1 was administered twice daily (at 9:00 a.m. and 5:00 p.m., 400 μg/kg daily dose) by subcutaneous injection, and 20 mg/kg body weight rosiglitazone was administered once daily by gavage at 9:00 a.m. Lean mice and db/db mice also received subcutaneous injections of saline as a control. The treatment groups were as follows:
1. lean mice with saline injection (control)
2. db/db mice with saline injection (control)
3. db/db mice with rosiglitazone (20 mg/kg, once daily)
4. db/db mice with rosiglitazone (20 mg/kg, once daily)+Eno1 (200 μg/kg, twice daily)

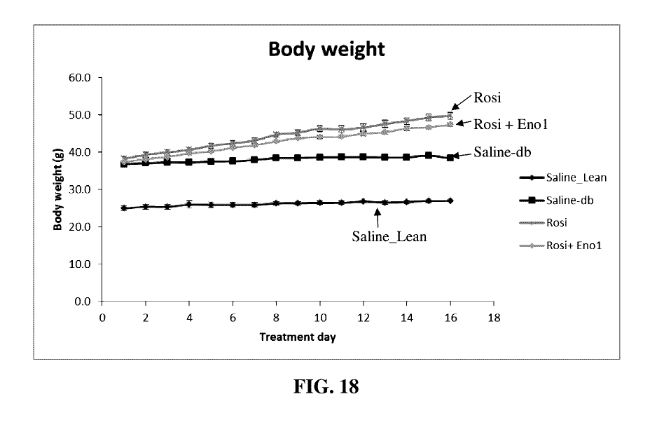

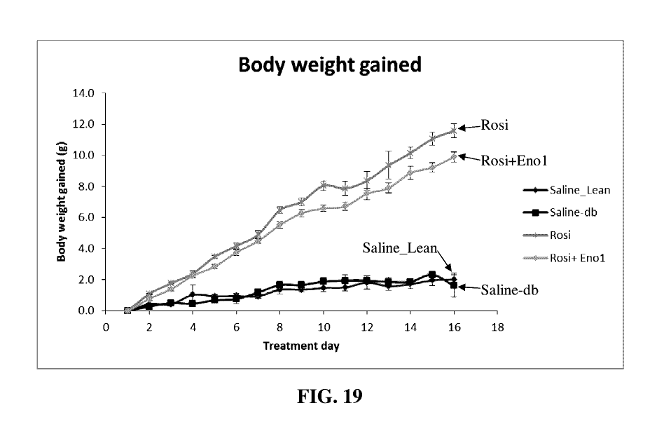

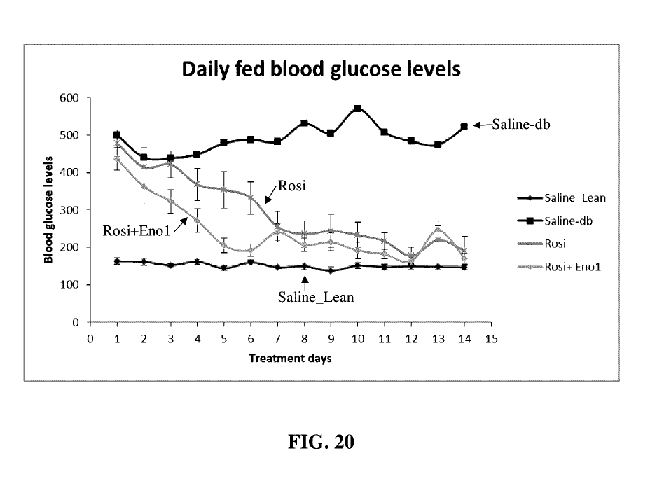

The mice were weighed daily to determine the effect of rosiglitazone and Eno1 on body weight gain. As shown in FIGS. 18 and 19, rosiglitazone alone and rosiglitazone+Eno1 showed increased body weight compared to control (saline treated) db/db mice. However, body weight was lower in the rosiglitazone+Eno1 treatment group compared to rosiglitazone alone, indicating that Eno1 attenuates rosiglitazone-induced weight gain.

The effect of Eno1 on lowering fed blood glucose was also tested in the db/db mice. Specifically, without controlling the intake of food, blood glucose levels in mice were measured once per day in the morning immediately before Eno1 and/or rosiglitazone treatment. The combination of rosiglitazone and Eno1 reduced blood glucose levels more quickly than rosiglitazone alone (FIG. 20).

While not wishing to be bound by theory, it is likely that muscle-targeted Eno1 limits glucose mediated fat storage in adipose tissue typically induced by rosiglitazone treatment by diverting some glucose to skeletal muscle for utilization (i.e. oxidation).

### Example 8

### Generation of a Detectably Labeled PAMAM Dendrimer, Muscle Targeted Eno1

A detectably labeled muscle targeted Eno1 was generated to analyze its efficacy in targeting to muscle cells. Detectably labeled G5-PAMAM dendrimers containing the muscle targeting peptide (MTP) ASSLNIA (SEQ ID NO: 7) and/or Eno1 were generated using the methods described below. A range of different ratios of MTP to dendrimer were evaluated, including MTP containing dendrimers which contained about 10 MTP peptides per dendrimer, about 3 MTP peptides per dendrimer, or about 1 MTP peptide per dendrimer.

The process of preparing Eno1 dendrimer complexes includes the identification of optimal ratios and concentrations of the reagents. Stock solutions of Eno1 were prepared in buffer and the protein solution was mixed with G5 dendrimer-muscle targeting peptide (MTP) conjugate in different ratios. A range of different ratios of dendrimer to Eno1 were also evaluated, including Eno1 containing dendrimers which contained about one dendrimer per molecule of Eno1 protein or about five dendrimers per molecule of Eno1 protein.

The stability of the Eno1-dendrimer-SMTP complex was evaluated at different temperatures, and stability was determined over a 3-4 month time period by measuring Eno1 activity using a commercially available Eno1 assay. The selected conjugates were also evaluated using biophysical techniques, including Dynamic Light Scattering (DLS) and UV-Vis spectroscopy to confirm complexation between the dendrimer-peptide conjugate and Eno1.

Determination of the Purity of Eno1:

The purity of a 5.32 mg/mL solution of Eno1 protein was checked by Coomassie and Silver staining and Western blotting. Several dilutions of the Eno1 protein ranging from 10 μg/well to 100 ng/well were prepared and loaded on a 12-well, 4-12% mini-PROTEAN® TGX gel [BIO-RAD Cat#456-1095 Lot#4000 79200]. The lane assignments were as follows; Lane 1: Ladder (Precision Plus Protein Standard Dual Color [BIO-RAD Cat#161-0374]; Lane 2: Eno1 (10.0 μg); Lane 3: Eno1 (1.0 μg); Lane 4: Eno1 (0.1 μg); Lane 5: Ladder (Precision Plus Protein Standard Dual Color [BIO-RAD Cat#161-0374]; Lane 6: Eno1 (10.0 μg); Lane 7: Eno1 (1.0 μg); Lane 8: Eno1 (0.1 μg); Lane 9: Ladder (Precision Plus Protein Standard Dual Color [BIO-RAD Cat#161-0374]; Lane 10: Eno1 (10.0 μg); Lane 11: Eno1 (1.0 μg); Lane 12: Eno1 (0.1 μg). The SDS-PAGE was run at 200 V for 20-25 min

Coomassie Staining:

After the gel was run, the gel was split into 3 equal parts. One of the parts was stained with Coomassie Stain. Briefly, the gel was soaked in 100 mL of Coomassie Stain solution (0.025% Coomassie Stain in 40% Methanol and 7% Acetic Acid) and heated for one minute in a microwave. Then the gel was left to stain with gentle agitation for 45 minutes. After the staining was complete, the gel was destained using destaining solution (40% Methanol and 7% Acetic Acid) until the background staining was acceptable. The protein ran as a single band of about 47 KDa, which is consistent with the size of Eno1.

Silver Staining:

Since Coomassie Staining is not a sensitive method for visualization of the protein bands, another portion of the gel was stained with Silver Stain using BIO-RAD's Silver Staining Kit [BIO-RAD Cat#161-0443]. The Modified Silver Stain Protocol was followed. Coomassie staining indicated that overall purity of the Eno1 was relatively high.

Western Blot Analysis:

The identity of Eno1 was further confirmed by Western blot. For this purpose, the final portion of the gel was transferred into 100 mL of Tris-Glycine buffer and transferred onto 0.2 μm PVDF membrane (BIO-RAD) using a transblot SD semi-dry transfer apparatus (BIO-RAD) at 20 V for 2.0 h. The efficiency of the transfer was checked by observing the presence of the pre-stained ladder bands on the membrane. The membrane was dried for 1.0 h. The membrane was then wetted with methanol for 1.0 min and blocked with 15.0 mL ODYSSEY® Blocking Buffer (LICOR) at room temperature for 2.0 h.

After the blocking was complete, the membrane was incubated with 15.0 mL ODYSSEY® Blocking Buffer containing 30 μL of anti-ENOA-1 m-Ab (mouse) (purchased from ABNOVA) overnight at 4° C. Then the membrane was washed with 3×30 mL of 1×PBS-T with shaking for 5 minutes each. The membrane was incubated with 15.0 mL ODYSSEY® Blocking Buffer containing 5 μL of Goat anti-mouse secondary antibody labeled with IRDye® 800CW (purchased from LICOR) for 2.0 h at room temperature. After the incubation, the membrane was washed with 3×30 mL of 1×PBS-T followed by 2×30 mL of 1×PBS with shaking for 5 minutes each. Finally, the membrane was imaged using the LICOR ODYSSEY Infrared Imager Western Blot analysis confirmed that the dominant band at 47 kDa was Eno1.

Zeta (ζ)-Potential Characterization of Enolase-I/G5-PAMAM-SMTP:

Eno1 and Generation 5 PAMAM dendrimers decorated with 2-3 Skeletal Muscle Targeting Peptides (SMTPs) were complexed at varied ratios to form Eno1/G5-SMTP protein/dendrimer complexes. The concentration of the dendrimer was kept constant at 1.0 μM and the Eno1 concentration was varied between 0.1 μM-10.0 μM. Table 3 below describes how the Enolase-I/G5-dendrimer/SMTP mixtures were prepared.

TABLE 3
Various combinations of Eno1 and G5-dendrimer/SMTP
for formation of dendrimer complexes.

| | | G5-Dendrimer | |
|---|---|---|---|
| Eno1/Dendrimer | Eno1 | SMTP | PBS buffer |
| Molar Ratio | (5.32 mg/mL) | (30.0 mg/mL) | pH = 7.40 |
| 10:1 | 88.3 μL | 1.03 μL | 910.67 μL |
| 5:1 | 44.15 μL | 1.03 μL | 954.82 μL |
| 2:1 | 17.66 μL | 1.03 μL | 981.31 μL |
| 1:1 | 8.83 μL | 1.03 μL | 990.14 μL |
| 1:2 | 4.42 μL | 1.03 μL | 994.55 μL |
| 1:5 | 1.77 μL | 1.03 μL | 997.2 μL |
| 1:10 | 0.88 μL | 1.03 μL | 998.09 μL |

Each sample was prepared by adding G5-dendrimer/SMTP to the respective amount of PBS. Enolase was then added to the G5-dendrimer/SMTP solution in a drop wise fashion while vortexing at low speed. The sample was then incubated at room temperature for 20 minutes prior to analysis.

Size measurements were made using the Zetasizer Nano Z90s instrument from Malvern Instruments. The default parameters were used for the measurements and three separate measurements of each sample were collected. Zeta (ζ)-Potential data for three samples of Eno1/G5-dendrimer/SMTP complexes having a 2:1 molar ratio of Eno1 to dendrimer/SMTP were collected. Zeta (ζ)-Potential was measured using Dynamic Light Scattering. The peaks of the three samples matched, indicating a uniform charge distribution of the Enolase-SMTP dendrimer complex.

Stability of Enolase-I/G5-SMTP Complexes:

The stability of the Enolase-1/G5-dendrimer/SMTP conjugates was measured by using the ENO1 Human Activity Assay Kit (ABCAM, Cambridge, Mass.; Catalogue No. ab117994). Briefly, the sample was added to a microplate containing a monoclonal mouse antibody specific to Eno1. The microplate was incubated at room temperature for 2 hours, and Eno1 was immunocaptured within the wells of the microplate. The wells of the microplate were washed to remove all other enzymes. Eno1 activity was determined by following the consumption of NADH in an assay buffer that included pyruvate kinase (PK), lactate dehydrogenase (LDH) and the required substrates 2-phospho-D-glycerate (2PG) and NADH. Eno1 converts 2PG to phosphoenolpyruvate, which is converted to pyruvate by PK. Pyruvate is converted to lactate by LDH, and this reaction requires NADH. The consumption of NADH was monitored as decrease of absorbance at 340 nm

The activity of Enolase-I/G5-dendrimer/SMTP conjugates that were stored at different temperatures at different time points was measured using the assay described above. A concentration of 500 ng of Eno1 was selected for testing because this concentration falls in the middle of the dynamic range of the assay kit. Two different sets of solutions were prepared. One set (control) contained Eno1 alone (i.e. unconjugated Eno1) and the other set contained Eno1/G5-dendrimer/SMTP mixtures. These mixtures were then kept at −80° C., −20° C., 4° C., 22° C., and 37° C. The results showed that in the first week all of the samples were active, and the Eno1/G5-dendrimer/SMTP conjugates seemed to have a slightly higher activity than Eno1 alone. However, the activities of the solutions, regardless of whether or not they contained dendrimers, steadily decreased in the next two weeks. By week 3, the solutions that were stored at 4° C., 22° C., and 37° C. showed no activity, while the solutions that were stored at −80° C., and −20° C. showed significant stability. At the end of the study (Week 10), The Eno1/G5-dendrimer/SMTP solution that was kept at −80° C. retained about 90% of its activity whereas Eno1 alone was only 35% active. On the other hand, Eno1/G5-dendrimer/SMTP solution that was kept at −20° C. was about 24% active, whereas Eno1 alone stored at −20° C. was not active.

### Example 9

### In Vivo Eno1 Targeting Studies with G5 PAMAM Dendrimers

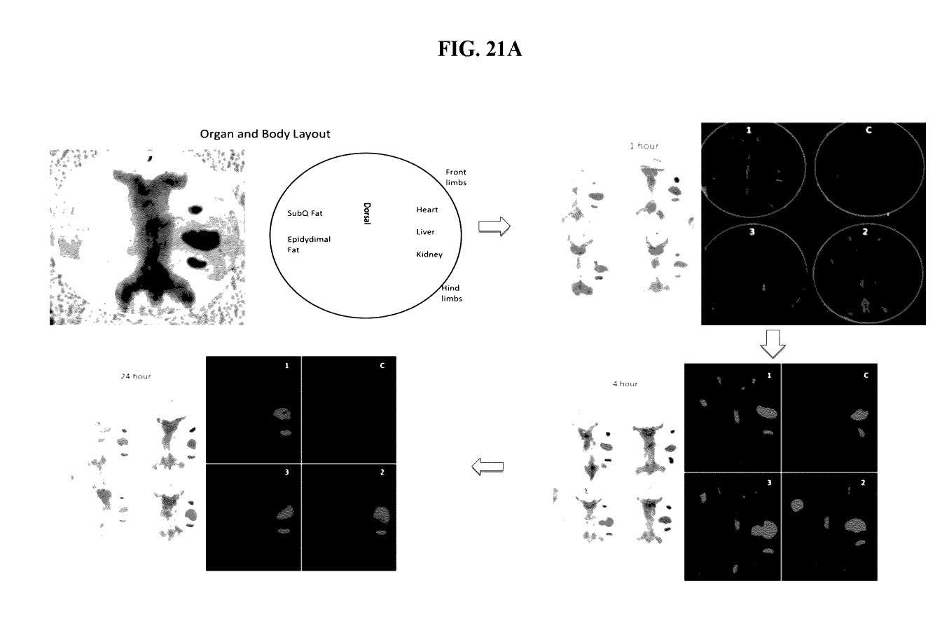

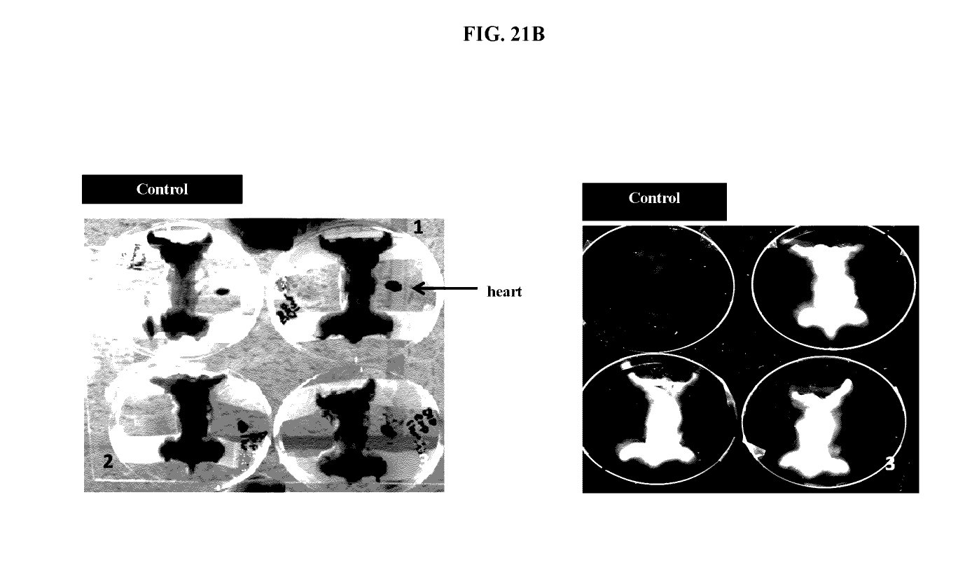

A detectably labeled PAMAM dendrimer complex containing Eno1 was prepared using the method provided in the prior example and analyzed for tissue distribution in mice after subcutaneous injection. Specifically, for 72 hours prior to injection mice were fed alfalfa free food to limit background fluorescence. Mice were injected with 3 μg ENO1/mouse subcutaneously 150 μl total (75 μl left laterally, 75 μl right laterally). The molar ratio of dendrimer to Eno1 in the complex was 5:1. One, 4, and 24 hours post injection animals were sacrificed, skinned, and organs removed in preparation for LI-COR imaging. The results are shown in FIG. 21A.

As shown, at 1 hour, general systemic distribution of the Eno1-PAMAM dendrimer was observed. After 4 hours, significant accumulation of the Eno1-PAMAM dendrimer was observed in liver, kidney, and subcutaneous fat, as well as in the upper torso. After 24 hours, the Eno1-dendrimer complex was substantially cleared and observed substantially in the liver and kidney.

A follow-up study was performed using the skeletal muscle targeted Eno1-PAMAM dendrimer complex containing the SMTP “ASSLNIA” (SEQ ID NO: 7). A detectably labeled PAMAM dendrimer complex containing Eno1 and SMTP ((Enolase-Vivo Tag680×1)-(G5-SMTP)) was prepared using the method provided in the prior example. The molar ratio of dendrimer to SMTP in the complex was 1:1. The experiments were performed essentially as described above. The skeletal muscle targeted Eno1-PAMAM dendrimer complex was administered at a dose of 50 μg/kg body weight. These images in FIG. 21B were taken after 1 hr of injection. Organs, other than the heart, were retained in the body. As can be readily observed, the muscle-targeted Eno1 dendrimer complex was targeted to skeletal muscle, not heart. These results demonstrate that the skeletal muscle targeted Eno1-PAMAM dendrimer complex can be used for the delivery of Eno1 to skeletal muscle cells.

### Example 9

### Effect of PEGylated Cysteine Modified Eno1 on HbA1c Levels in db/db Mice (Prophetic)

Glycated hemoglobin (hemoglobin A1c, HbA1c, A1C, Hb1c, HbA1c) is a form of hemoglobin that is measured primarily to identify the average plasma glucose concentration over prolonged periods of time, i.e., an indirect measurement of blood glucose. HbA1c is formed in a non-enzymatic glycation pathway by hemoglobin's exposure to plasma glucose. When blood glucose levels are high, elevated levels of glycated hemoglobin are produced. Glycation is an irreversible reaction. Therefore, the amount of glycated hemoglobin within the red blood cell reflects the average level of glucose to which the cell has been exposed.

HbA1c levels will be measured in db/db mice treated with pegylated, cysteine-modified, muscle-targeted Eno1 fusion proteins as described in Example 6 above. HbA1c levels can be measured, for example, using high-performance liquid chromatography (HPLC) or immunoassay. Methods for detection and measurement of HbA1c are routine in the art and are described, for example, in Hoshino et al., 1990, J. Chromatography 515: 531-536, which is incorporated by reference herein in its entirety.

It is expected that administration of the Eno1 fusion proteins to db/db mice will reduce HbA1c levels relative to mice that are not treated with the fusion proteins.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

### INCORPORATION BY REFERENCE

Each reference, patent, patent application, and GenBank number referred to in the instant application is hereby incorporated by reference as if each reference were noted to be incorporated individually.

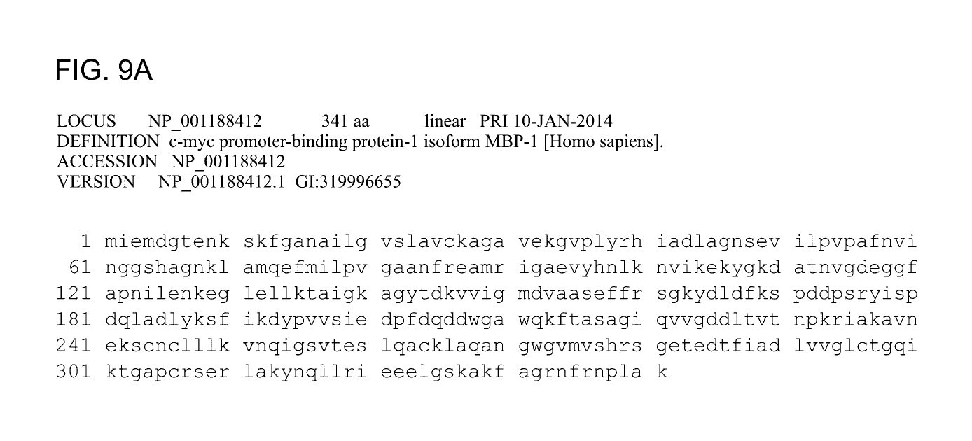

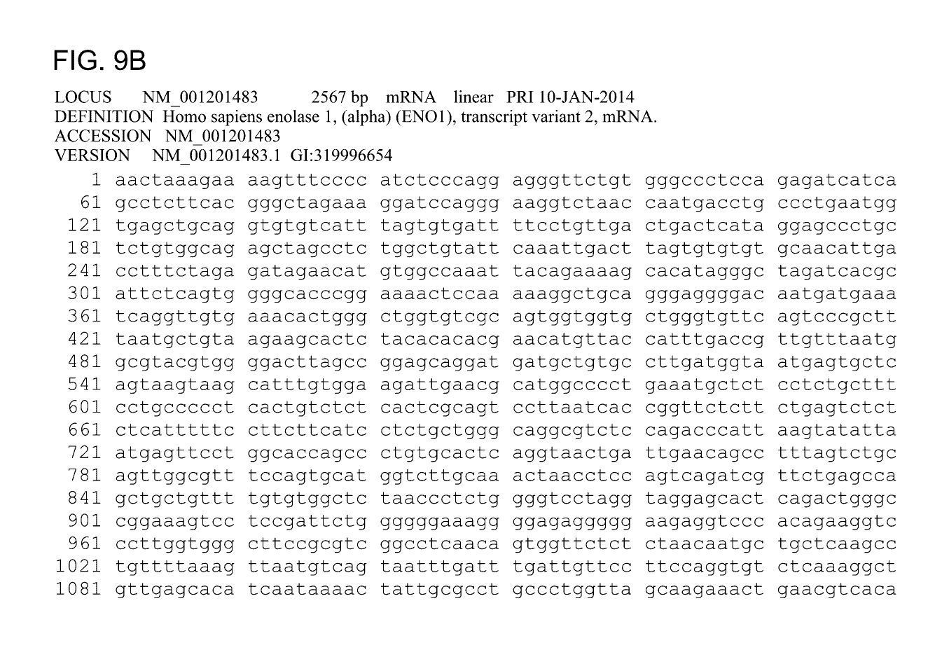

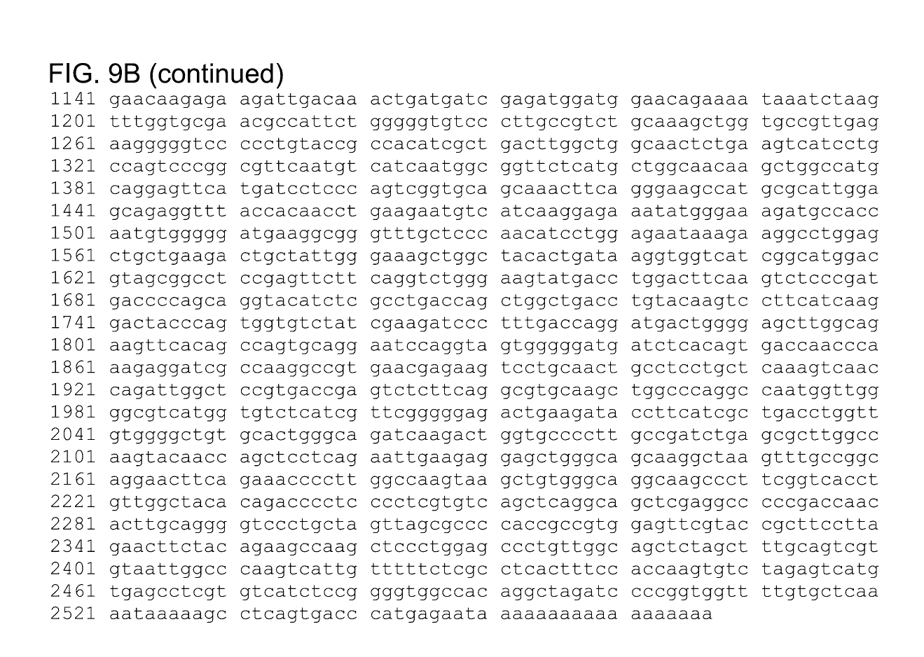

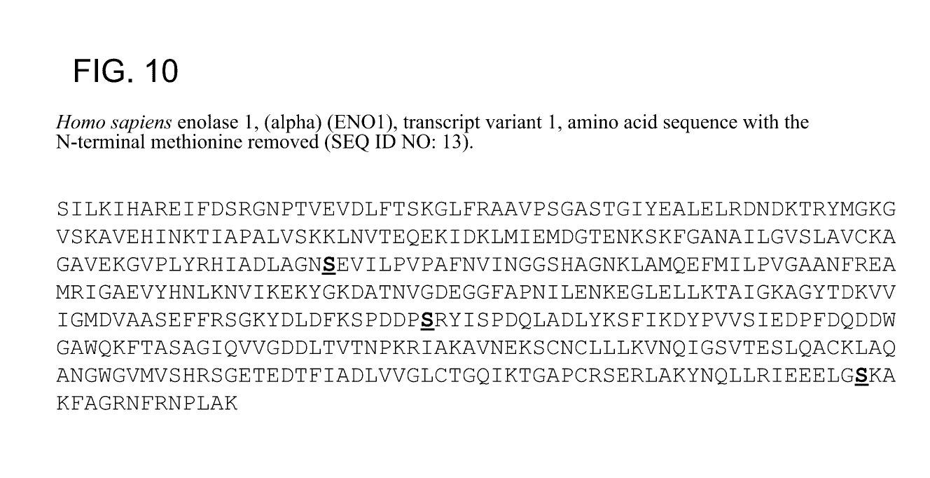

TABLE 3
Description of Sequences

| SEQ ID | | |
|---|---|---|
| NO: | Sequence | Description |
| 1 | DNA | Human Eno1, transcript |
| | | variant 1. (FIG. 8B) |
| 2 | AA | Human Eno1, transcript |
| | | variant 1. (FIG. 8A) |
| 3 | DNA | Human Eno1, transcript |
| | | variant 2. (FIG. 9B) |
| 4 | AA | Human Eno1, transcript |
| | | variant 2, also referred |
| | | to as c-myc promoter- |
| | | binding protein-1 |
| | | (MBP-1). (FIG. 9A) |
| 5 | AA | Eno1 fusion protein |
| | | comprising an N-terminal |
| | | muscle targeting peptide |
| | | (MTP) (ASSLNIA, SEQ ID |
| | | NO: 7), a protease tag |
| | | (SSGVDLGTENLYFQ, SEQ ID |
| | | NO: 6), and human Eno1, |
| | | transcript variant 1 with |
| | | the N-terminal methionine |
| | | removed (SEQ ID NO: 13, |
| | | FIG. 11). |
| 6 | AA | Protease tag comprising a |
| | | Tobacco Etch Virus (TEV) |
| | | protease cleavage site |
| | | (SSGVDLGTENLYFQ). The TEV |
| | | protease cleavage site is |
| | | underlined. |
| 7 | AA | muscle targeting peptide |
| | | (ASSLNIA) |
| 8 | AA | muscle targeting peptide |
| | | (WDANGKT) |
| 9 | AA | muscle targeting peptide |
| | | (GETRAPL) |
| 10 | AA | muscle targeting peptide |
| | | (CGHHPVYAC) |
| 11 | AA | muscle targeting peptide |
| | | (HAIYPRH) |
| 12 | AA | TEV protease cleavage |
| | | site (ENLYFQ) |
| 13 | AA | Human Eno1, transcript |
| | | variant 1, with the |
| | | N-terminal methionine |
| | | removed (FIG. 10) |
| 14 | AA | Glycine-Serine Linker |
| | | (GGSGGSGGSGGSGGS) |
| 15 | AA | Peptide added to |
| | | C-terminus of Eno1 |
| | | (GIEGR) |
| 16 | AA | Cysteine modified Eno1 |
| | | fusion protein S267C |
| | | (FIG. 17) |
| 17 | AA | Cysteine modified Eno1 |
| | | fusion protein S140C |
| 18 | AA | Cysteine modified Eno1 |
| | | fusion protein S418C |
| 19 | AA | Cysteine modified Eno1 |
| | | fusion protein S140C/ |
| | | S267C/S418C |
| 20 | AA | Cell penetrating peptide |
| 21 | AA | Cell penetrating peptide |
| 22 | AA | Cell penetrating peptide |
| 23 | AA | Cell penetrating peptide |

We claim:

1. An Eno1 molecule comprising an Eno1 polypeptide or a fragment thereof and a muscle targeting peptide, wherein the Eno1 polypeptide or fragment thereof is covalently attached to the muscle targeting peptide.

2. The Eno1 molecule of claim 1, wherein the molecule is for delivery to a muscle cell.

3. The Eno1 molecule of claim 1, wherein the Eno1 polypeptide or fragment thereof is biologically active.

4. The Eno1 molecule of claim 1, wherein the Eno1 polypeptide or fragment thereof has at least 90% of the activity of a purified endogenous human Eno1 polypeptide.

5. The Eno1 molecule of claim 1, wherein the Eno1 polypeptide or fragment thereof is human Eno1 or a fragment thereof.

6. The Eno1 molecule of claim 1, wherein the muscle targeting peptide comprises an amino acid sequence selected from the group consisting of: ASSLNIA (SEQ ID NO: 7); WDANGKT (SEQ ID NO: 8); GETRAPL (SEQ ID NO: 9); CGHHPVYAC (SEQ ID NO: 5); and HAIYPRH (SEQ ID NO: 6).

7. The Eno1 molecule of claim 1, wherein the Eno1 molecule further comprises a linker.

8-10. (canceled)

11. The Eno1 molecule of claim 7, wherein the linker is a peptide comprising a protease cleavage site.

12. The Eno1 molecule of claim 7, wherein the linker comprises the amino acid sequence of SEQ ID NO: 6.

13. The Eno1 molecule of claim 1, wherein the Eno1 polypeptide or fragment thereof and the muscle targeting peptide are comprised in a single polypeptide.

14. The Eno1 molecule of claim 1, further comprising one or more functional moiety.

15. The Eno1 molecule of claim 14, wherein the Eno1 polypeptide or fragment thereof is covalently attached to the one or more functional moiety.

16. The Eno1 molecule of claim 14, wherein the Eno1 polypeptide, or fragment thereof, comprises one or more cysteine residues covalently attached to the one or more functional moiety.

17-18. (canceled)

19. The Eno1 molecule of claim 16, wherein the cysteine residues are added cysteine residues.

20. The Eno1 molecule of claim 16, wherein the cysteine residues are at a position selected from the group consisting of position 26, 78, 140, 236, 253, 267 and 418 of the amino acid sequence of SEQ ID NO: 13.

21. The Eno1 molecule of claim 1, wherein the Eno1 polypeptide or fragment thereof is released from the muscle targeting peptide or the one or more functional moiety upon delivery to a muscle cell.

22. The Eno1 molecule of claim 14, wherein the one or more functional moiety is a moiety selected from the group consisting of a biocompatible polymer, a cell penetrating peptide, and a muscle targeting peptide.

23. The Eno1 molecule of claim 14, wherein the functional moiety is a biocompatible polymer.

24. The Eno1 molecule of claim 23, wherein the biocompatible polymer comprises polyethylene glycol (PEG).

25. The Eno1 molecule of claim 24, wherein the PEG is a linear PEG or a branched PEG.

26. The Eno1 molecule of claim 24, wherein the PEG is a 5 kDa PEG, 10 kDa PEG, or 20 kDa PEG.

27. The Eno1 molecule of claim 13, wherein the single polypeptide comprises the amino acid sequence of SEQ ID NO: 16 comprising an added cysteine residue at position 289, wherein the added cysteine residue at position 289 is covalently linked to at least one PEG molecule.

28. The Eno1 molecule of claim 27, wherein the added cysteine residue is covalently linked to the PEG molecule through a maleimide linkage.

29. A pharmaceutical composition comprising the Eno1 molecule of claim 1.

30. A nucleic acid encoding the Eno1 molecule of claim 1.

31. An expression vector comprising the nucleic acid of claim 30.

32. An Eno1 molecule comprising an Eno1 polypeptide or a fragment thereof, wherein the Eno1 polypeptide or fragment thereof comprises at least one added cysteine residue.

33-34. (canceled)

35. The Eno1 molecule of claim 32, wherein the added cysteine residue is added to the N-terminus of the Eno1 polypeptide or fragment thereof.

36. The Eno1 molecule of claim 32, wherein the added cysteine residue replaces an internal serine or threonine of the Eno1 polypeptide or fragment thereof.

37. The Eno1 molecule of claim 36, wherein the added cysteine residue is at one or more positions selected from the group consisting of position 26, 78, 140, 236, 253, 267 and 418 of the amino acid sequence of SEQ ID NO: 13.

38. The Eno1 molecule of claim 32, wherein the Eno1 molecule further comprises a functional moiety.

39. (canceled)

40. The Eno1 molecule of claim 38, wherein the functional moiety is a muscle targeting peptide.

41. The Eno1 molecule of claim 40, wherein the muscle targeting peptide comprises an amino acid sequence selected from the group consisting of: ASSLNIA (SEQ ID NO: 7); WDANGKT (SEQ ID NO: 8); GETRAPL (SEQ ID NO: 9); CGHHPVYAC (SEQ ID NO: 5); and HAIYPRH (SEQ ID NO: 6).

42. The Eno1 molecule of claim 40, wherein the Eno1 polypeptide or fragment thereof and the muscle targeting peptide are comprised in a single polypeptide.

43. The Eno1 molecule of claim 38, wherein the Eno1 molecule comprises a polypeptide linker between the Eno1 polypeptide or fragment thereof and the muscle targeting peptide.

44. The Eno1 molecule of claim 43, wherein the polypeptide linker comprises the amino acid sequence of SEQ ID NO. 6.

45. The Eno1 molecule of claim 38, wherein the functional moiety is a biocompatible polymer.

46. The Eno1 molecule of claim 45, wherein the biocompatible polymer comprises polyethylene glycol (PEG).

47. The Eno1 molecule of claim 46, wherein the PEG is a linear PEG or a branched PEG.

48. The Eno1 molecule of claim 46, wherein the PEG is a 5 kDa PEG, 10 kDa PEG, or 20 kDa PEG.

49. The Eno1 molecule of claim 38, wherein the Eno1 molecule comprises a linker between the functional moiety and the Eno1 polypeptide or fragment thereof.

50. The Eno1 molecule of claim 49, wherein the linker is attached to the Eno1 polypeptide or fragment thereof at the added cysteine residue.

51. The Eno1 molecule of claim 7, wherein the linker comprises the amino acid sequence of SEQ ID NO: 14.

52. The Eno1 molecule of claim 43, wherein the N-terminus of the linker is attached to the Eno1 polypeptide or fragment thereof at the added cysteine residue.

53. The Eno1 molecule of claim 42, wherein the single polypeptide comprises the amino acid sequence of SEQ ID NO: 16 comprising an added cysteine residue at position 289, wherein the added cysteine residue at position 289 is covalently linked to at least one PEG molecule through a maleimide linkage.

54. The Eno1 molecule of claim 53, wherein the at least one PEG molecule is a linear 20 kDa PEG.

55. A pharmaceutical composition comprising the Eno1 molecule of claim 32.

56. A nucleic acid encoding the Eno1 molecule of claim 32.

57. An expression vector comprising the nucleic acid of claim 56.

58. The pharmaceutical composition of claim 29, wherein the composition is formulated for parenteral administration.

59. The pharmaceutical composition of claim 29, wherein the composition is formulated for oral administration.

60. The pharmaceutical composition of claim 29, wherein the composition is formulated for intramuscular administration, intravenous administration, or subcutaneous administration.

61. A method of decreasing blood glucose in a subject with elevated blood glucose, the method comprising administering to the subject the pharmaceutical composition of claim 29, thereby decreasing blood glucose in the subject.

62. A method of increasing glucose tolerance in a subject with decreased glucose tolerance, the method comprising administering to the subject the pharmaceutical composition of claim 29, thereby increasing glucose tolerance in the subject.

63. A method of improving insulin response in a subject with decreased insulin sensitivity and/or insulin resistance, the method comprising administering to the subject the pharmaceutical composition of claim 29, thereby improving insulin response in the subject.

64. A method of treating diabetes in a subject, the method comprising administering to the subject the pharmaceutical composition of claim 29, thereby treating diabetes in the subject.

65-66. (canceled)

67. A method of decreasing an HbA1c level in a subject with an elevated Hb1Ac level, the method comprising administering to the subject the pharmaceutical composition of claim 29, thereby decreasing the HbA1c level in the subject.

68-78. (canceled)


SEQUENCE LISTING (23 sequences)

SEQ ID NO: 1 - DNA, 2204 bp, Homo sapiens
gtggggccccagagcgacgctgagtgcgtgcgggactcggagtacgtgacggagccccgagctctcatgcccgccacgccgccccgggccatcccccggagccccggctccgcacaccccagttcggctcaccggtcctatctggggccagagtttcgcccgcaccactacagggccgctggggagtcggggccccccagatctgcccgcctcaagtccgcgggacgtcacccccctttccacgctactgcagccgtcgcagtcccacccctttccgggaggtgagggaatgagtgacggctctcccgacgaatggcgaggcggagctgagggggcgtgccccggaggcgggaagtgggtggggctcgccttagctaggcaggaagtcggcgcgggcggcgcggacagtatctgtgggtacccggagcacggagatctcgccggctttacgttcacctcggtgtctgcagcaccctccgcttcctctcctaggcgacgagacccagtggctagaagttcaccatgtctattctcaagatccatgccagggagatctttgactctcgcgggaatcccactgttgaggttgatctcttcacctcaaaaggtctcttcagagctgctgtgcccagtggtgcttcaactggtatctatgaggccctagagctccgggacaatgataagactcgctatatggggaagggtgtctcaaaggctgttgagcacatcaataaaactattgcgcctgccctggttagcaagaaactgaacgtcacagaacaagagaagattgacaaactgatgatcgagatggatggaacagaaaataaatctaagtttggtgcgaacgccattctgggggtgtcccttgccgtctgcaaagctggtgccgttgagaagggggtccccctgtaccgccacatcgctgacttggctggcaactctgaagtcatcctgccagtcccggcgttcaatgtcatcaatggcggttctcatgctggcaacaagctggccatgcaggagttcatgatcctcccagtcggtgcagcaaacttcagggaagccatgcgcattggagcagaggtttaccacaacctgaagaatgtcatcaaggagaaatatgggaaagatgccaccaatgtgggggatgaaggcgggtttgctcccaacatcctggagaataaagaaggcctggagctgctgaagactgctattgggaaagctggctacactgataaggtggtcatcggcatggacgtagcggcctccgagttcttcaggtctgggaagtatgacctggacttcaagtctcccgatgaccccagcaggtacatctcgcctgaccagctggctgacctgtacaagtccttcatcaaggactacccagtggtgtctatcgaagatccctttgaccaggatgactggggagcttggcagaagttcacagccagtgcaggaatccaggtagtgggggatgatctcacagtgaccaacccaaagaggatcgccaaggccgtgaacgagaagtcctgcaactgcctcctgctcaaagtcaaccagattggctccgtgaccgagtctcttcaggcgtgcaagctggcccaggccaatggttggggcgtcatggtgtctcatcgttcgggggagactgaagataccttcatcgctgacctggttgtggggctgtgcactgggcagatcaagactggtgccccttgccgatctgagcgcttggccaagtacaaccagctcctcagaattgaagaggagctgggcagcaaggctaagtttgccggcaggaacttcagaaaccccttggccaagtaagctgtgggcaggcaagcccttcggtcacctgttggctacacagacccctcccctcgtgtcagctcaggcagctcgaggcccccgaccaacacttgcaggggtccctgctagttagcgccccaccgccgtggagttcgtaccgcttccttagaacttctacagaagccaagctccctggagccctgttggcagctctagctttgcagtcgtgtaattggcccaagtcattgtttttctcgcctcactttccaccaagtgtctagagtcatgtgagcctcgtgtcatctccggggtggccacaggctagatccccggtggttttgtgctcaaaataaaaagcctcagtgacccatgagaataaaaaaaaaaaaaaaaaa

SEQ ID NO: 2 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 3 - DNA, 2567 bp, Homo sapiens
aactaaagaaaagtttccccatctcccaggagggttctgtgggccctccagagatcatcagcctcttcacgggctagaaaggatccagggaaggtctaaccaatgacctgccctgaatggtgagctgcaggtgtgtcatttagtgtgattttcctgttgactgactcataggagccctgctctgtggcagagctagcctctggctgtattcaaattgacttagtgtgtgtgcaacattgacctttctagagatagaacatgtggccaaattacagaaaagcacatagggctagatcacgcattctcagtggggcacccggaaaactccaaaaaggctgcagggaggggacaatgatgaaatcaggttgtgaaacactgggctggtgtcgcagtggtggtgctgggtgttcagtcccgctttaatgctgtaagaagcactctacacacacgaacatgttaccatttgaccgttgtttaatggcgtacgtggggacttagccggagcaggatgatgctgtgccttgatggtaatgagtgctcagtaagtaagcatttgtggaagattgaacgcatggcccctgaaatgctctcctctgctttcctgccccctcactgtctctcactcgcagtccttaatcaccggttctcttctgagtctctctcatttttccttcttcatcctctgctgggcaggcgtctccagacccattaagtatattaatgagttcctggcaccagccctgtgcactcaggtaactgattgaacagcctttagtctgcagttggcgtttccagtgcatggtcttgcaaactaacctccagtcagatcgttctgagccagctgctgttttgtgtggctctaaccctctggggtcctaggtaggagcactcagactgggccggaaagtcctccgattctggggggaaaggggagagggggaagaggtcccacagaaggtcccttggtgggcttccgcgtcggcctcaacagtggttctctctaacaatgctgctcaagcctgttttaaagttaatgtcagtaatttgatttgattgttccttccaggtgtctcaaaggctgttgagcacatcaataaaactattgcgcctgccctggttagcaagaaactgaacgtcacagaacaagagaagattgacaaactgatgatcgagatggatggaacagaaaataaatctaagtttggtgcgaacgccattctgggggtgtcccttgccgtctgcaaagctggtgccgttgagaagggggtccccctgtaccgccacatcgctgacttggctggcaactctgaagtcatcctgccagtcccggcgttcaatgtcatcaatggcggttctcatgctggcaacaagctggccatgcaggagttcatgatcctcccagtcggtgcagcaaacttcagggaagccatgcgcattggagcagaggtttaccacaacctgaagaatgtcatcaaggagaaatatgggaaagatgccaccaatgtgggggatgaaggcgggtttgctcccaacatcctggagaataaagaaggcctggagctgctgaagactgctattgggaaagctggctacactgataaggtggtcatcggcatggacgtagcggcctccgagttcttcaggtctgggaagtatgacctggacttcaagtctcccgatgaccccagcaggtacatctcgcctgaccagctggctgacctgtacaagtccttcatcaaggactacccagtggtgtctatcgaagatccctttgaccaggatgactggggagcttggcagaagttcacagccagtgcaggaatccaggtagtgggggatgatctcacagtgaccaacccaaagaggatcgccaaggccgtgaacgagaagtcctgcaactgcctcctgctcaaagtcaaccagattggctccgtgaccgagtctcttcaggcgtgcaagctggcccaggccaatggttggggcgtcatggtgtctcatcgttcgggggagactgaagataccttcatcgctgacctggttgtggggctgtgcactgggcagatcaagactggtgccccttgccgatctgagcgcttggccaagtacaaccagctcctcagaattgaagaggagctgggcagcaaggctaagtttgccggcaggaacttcagaaaccccttggccaagtaagctgtgggcaggcaagcccttcggtcacctgttggctacacagacccctcccctcgtgtcagctcaggcagctcgaggcccccgaccaacacttgcaggggtccctgctagttagcgccccaccgccgtggagttcgtaccgcttccttagaacttctacagaagccaagctccctggagccctgttggcagctctagctttgcagtcgtgtaattggcccaagtcattgtttttctcgcctcactttccaccaagtgtctagagtcatgtgagcctcgtgtcatctccggggtggccacaggctagatccccggtggttttgtgctcaaaataaaaagcctcagtgacccatgagaataaaaaaaaaaaaaaaaaa

SEQ ID NO: 4 - protein, 341 aa, Homo sapiens
MIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 5 - protein, 455 aa, Artificial Sequence
  ENO1 fusion protein
MASSLNIASSGVDLGTENLYFQSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 6 - protein, 14 aa, Artificial Sequence
  Linker
SSGVDLGTENLYFQ

SEQ ID NO: 7 - protein, 7 aa, Artificial Sequence
  Muscle targeting peptide
ASSLNIA

SEQ ID NO: 8 - protein, 7 aa, Artificial Sequence
  Muscle targeting peptide
WDANGKT

SEQ ID NO: 9 - protein, 7 aa, Artificial Sequence
  Muscle targeting peptide
GETRAPL

SEQ ID NO: 10 - protein, 9 aa, Artificial Sequence
  Muscle targeting peptide
CGHHPVYAC

SEQ ID NO: 11 - protein, 7 aa, Artificial Sequence
  Muscle targeting peptide
HAIYPRH

SEQ ID NO: 12 - protein, 6 aa, Artificial Sequence
  TEV protease cleavage site
ENLYFQ

SEQ ID NO: 13 - protein, 433 aa, Artificial Sequence
  ENO1 with N-terminal methionine removed
SILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 14 - protein, 15 aa, Artificial Sequence
  Linker
GGSGGSGGSGGSGGS

SEQ ID NO: 15 - protein, 5 aa, Artificial Sequence
  Peptide added to C-terminus of ENO1
GIEGR

SEQ ID NO: 16 - protein, 460 aa, Artificial Sequence
  S267C ENO1 fusion protein
MASSLNIASSGVDLGTENLYFQSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPCRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAKGIEGR

SEQ ID NO: 17 - protein, 460 aa, Artificial Sequence
  S140C Eno1 fusion protein
MASSLNIASSGVDLGTENLYFQSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNCEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAKGIEGR

SEQ ID NO: 18 - protein, 460 aa, Artificial Sequence
  S418C Eno1 fusion protein
MASSLNIASSGVDLGTENLYFQSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGCKAKFAGRNFRNPLAKGIEGR

SEQ ID NO: 19 - protein, 460 aa, Artificial Sequence
  S140C/S267C/S418C Eno1 fusion protein
MASSLNIASSGVDLGTENLYFQSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNCEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPCRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGCKAKFAGRNFRNPLAKGIEGR

SEQ ID NO: 20 - protein, 16 aa, Artificial Sequence
  Cell penetrating peptide
RQIKIWFQNRRMKWKK

SEQ ID NO: 21 - protein, 21 aa, Artificial Sequence
  Cell penetrating peptide
AGYLLGKINLKALAALAKKIL

SEQ ID NO: 22 - protein, 22 aa, Artificial Sequence
  Cell penetrating peptide
MVTVLFRRLRIRRACGPPRVRV

SEQ ID NO: 23 - protein, 8 aa, Artificial Sequence
  Cell penetrating peptide
YDEEGGGE